SEQUENCE LISTING (72 sequences)

SEQ ID NO: 1 - DNA, 28 bp, Artifificial Sequence
  Synthetic
cgcgggtaccaacggtgcattggaacgc

SEQ ID NO: 2 - DNA, 38 bp, Artificial Sequence
  Synthetic
atcggctagcggacggtgactgcagaaaagacccatgg

SEQ ID NO: 3 - DNA, 31 bp, Artificial Sequence
  Synthetic
atgcatgccgtgttgacaattaatcatcggc

SEQ ID NO: 4 - DNA, 30 bp, Artificial Sequence
  Synthetic
atgttaaccacgtgtcagtcctgctcctcg

SEQ ID NO: 5 - DNA, 30 bp, Artificial Sequence
  Synthetic
agccggtaccctattccagaagtagtgagg

SEQ ID NO: 6 - DNA, 30 bp, Artificial Sequence
  Synthetic
cgtaactcgagcactgcattctagttgtgg

SEQ ID NO: 7 - DNA, 30 bp, Artificial Sequence
  Synthetic
agccgctagcctattccagaagtagtgagg

SEQ ID NO: 8 - DNA, 26 bp, Artificial Sequence
  Synthetic
tattaagcgggggagaattttttttt

SEQ ID NO: 9 - DNA, 26 bp, Artificial Sequence
  Synthetic
aaaaaaaaattctcccccgcttaata

SEQ ID NO: 10 - DNA, 26 bp, Artificial Sequence
  Synthetic
caggtcagccaaaattaccttttttt

SEQ ID NO: 11 - DNA, 26 bp, Artificial Sequence
  Synthetic
aaaaaaaggtaattttggctgacctg

SEQ ID NO: 12 - DNA, 26 bp, Artificial Sequence
  Synthetic
gaaagattgttaagtgtttttttttt

SEQ ID NO: 13 - DNA, 26 bp, Artificial Sequence
  Synthetic
aaaaaaaaaactcttaacaatctttc

SEQ ID NO: 14 - DNA, 26 bp, Artificial Sequence
  Synthetic
aattctcccccgcttaataggggggg

SEQ ID NO: 15 - DNA, 26 bp, Artificial Sequence
  Synthetic
ccccccctattaagcgggggagaatt

SEQ ID NO: 16 - DNA, 26 bp, Artificial Sequence
  Synthetic
ggtaattttggctgacctgggggggg

SEQ ID NO: 17 - DNA, 26 bp, Artificial Sequence
  Synthetic
ccccccccaggtcagccaaaattacc

SEQ ID NO: 18 - DNA, 26 bp, Artificial Sequence
  Synthetic
aattctcccccgcttaataggggggg

SEQ ID NO: 19 - DNA, 26 bp, Artificial Sequence
  Synthetic
ccccccctattaagcgggggagaatt

SEQ ID NO: 20 - DNA, 130 bp, Artificial Sequence
  Synthetic
tattaagcgggggagaatttttttttcaggtcagccaaaattacctttttttgaaagattgttaagtgttttttttttggtaattttggctgacctggggggggaattctcccccgcttaataggggggg

SEQ ID NO: 21 - DNA, 59 bp, Artificial Sequence
  Synthetic
tcgacggtgcgttcctcgtagagaagatcaagagtcttctctacgaggaacgcaccgtg

SEQ ID NO: 22 - DNA, 61 bp, Artificial Sequence
  Synthetic
tgcacacacggtgcgttcctcgtagagaagactcttgatcttctctacgaggaacgcaccg

SEQ ID NO: 23 - DNA, 60 bp, Artificial Sequence
  Synthetic
tgtgcaaaggcgggaatgtctgcgtcaagagcgcagacattcccgcctttgcagtgtgga

SEQ ID NO: 24 - DNA, 60 bp, Artificial Sequence
  Synthetic
tagcgatccacactgcaaaggcgggaatgtctgcgctcttgacgcagacattcccgcctt

SEQ ID NO: 25 - DNA, 50 bp, Artificial Sequence
  Synthetic
tcgctattacaattcctcattcaagagatgaggaattgtaatagcgatct

SEQ ID NO: 26 - DNA, 48 bp, Artificial Sequence
  Synthetic
ctagagatcgctattacaattcctcatctcttgaatgaggaattgtaa

SEQ ID NO: 27 - DNA, 169 bp, Artificial Sequence
  Synthetic
tcgacggtgcgttcctcgtagagaagatcaagagtcttctctacgaggaacgcaccgtgtgtgcaaaggcgggaatgtctgcgtcaagagcgcagacattcccgcctttgcagtgtggatcgctattacaattcctcattcaagagatgaggaattgtaatagcgatct

SEQ ID NO: 28 - DNA, 107 bp, Artificial Sequence
  Synthetic
tcgagaaaattaattaaaaaacggccgaaaatctagaaaaaggtaccaaaagaattcaaaagctagcaaaagcggccgcaaaacgatcgaaaagtcgacaaaagttt

SEQ ID NO: 29 - DNA, 58 bp, Artificial Sequence
  Synthetic
tcgagttaagcgggggagaattagatggggggatctaattctcccccgcttaattaat

SEQ ID NO: 30 - DNA, 51 bp, Artificial Sequence
  Synthetic
taattaagcgggggagaattagatccccccatctaattctcccccgcttaa

SEQ ID NO: 31 - DNA, 54 bp, Artificial Sequence
  Synthetic
ggccgcaggtcagccaaaattaccctggggggagggtaattttggctgacctgt

SEQ ID NO: 32 - DNA, 54 bp, Artificial Sequence
  Synthetic
ctagacaggtcagccaaaattaccctccccccagggtaattttggctgacctgc

SEQ ID NO: 33 - DNA, 50 bp, Artificial Sequence
  Synthetic
ctgttttcagcattatcagaagggggggcttctgataatgctgaaaacag

SEQ ID NO: 34 - DNA, 58 bp, Artificial Sequence
  Synthetic
aattctgttttcagcattatcagaagcccccccttctgataatgctgaaaacaggtac

SEQ ID NO: 35 - DNA, 55 bp, Artificial Sequence
  Synthetic
ctagcataaaatagtaagaatgtataggggggtatacattcttactattttatgc

SEQ ID NO: 36 - DNA, 55 bp, Artificial Sequence
  Synthetic
ggccgcataaaatagtaagaatgtatacccccctatacattcttactattttatg

SEQ ID NO: 37 - DNA, 51 bp, Artificial Sequence
  Synthetic
cggaaagattgttaagtgtttcaggggggtgaaacacttaacaatctttcg

SEQ ID NO: 38 - DNA, 57 bp, Artificial Sequence
  Synthetic
tcgacgaaagattgttaagtgtttcacccccctgaaacacttaacaatctttccgat

SEQ ID NO: 39 - DNA, 10 bp, Artificial Sequence
  Synthetic
ctctctctct

SEQ ID NO: 40 - DNA, 11 bp, Artificial Sequence
  Synthetic
cttcttccttc

SEQ ID NO: 41 - DNA, 16 bp, Artificial Sequence
  Synthetic
ccctcccttcctcttc

SEQ ID NO: 42 - DNA, 9 bp, Artificial Sequence
  Synthetic
ttcaaaaga

SEQ ID NO: 43 - DNA, 11 bp, Artificial Sequence
  Synthetic
gggttctcttc

SEQ ID NO: 44 - DNA, 12 bp, Artificial Sequence
  Synthetic
catgtccatttt

SEQ ID NO: 45 - DNA, 11 bp, Artificial Sequence
  Synthetic
gggctcctctt

SEQ ID NO: 46 - DNA, 15 bp, Artificial Sequence
  Synthetic
ggtgtggtccctttt

SEQ ID NO: 47 - DNA, 11 bp, Artificial Sequence
  Synthetic
gggaccacacc

SEQ ID NO: 48 - DNA, 15 bp, Artificial Sequence
  Synthetic
aagaggagccctttt

SEQ ID NO: 49 - DNA, 123 bp, Artificial Sequence
  Synthetic
cgcgcctaatacgactcactatagggagaccacaacggtttccctctagcgggatcaaaaaaacgccgcagacacatccattcaagagatggatgtgtctgcggcgttttttatctgtttttc

SEQ ID NO: 50 - DNA, 123 bp, Artificial Sequence
  Synthetic
ctaggaaaaacagataaaaaacgccgcagacacatccatctcttgaatggatgtgtctgcggcgtttttttgatcccgctagagggaaaccgttgtggtctccctatagtgagtcgtattagg

SEQ ID NO: 51 - DNA, 21 bp, Artificial Sequence
  Synthetic
gtcttgccgcctgtgagtccg

SEQ ID NO: 52 - DNA, 21 bp, Artificial Sequence
  Synthetic
cgctaacacatctacctcaga

SEQ ID NO: 53 - DNA, 25 bp, Artificial Sequence
  Synthetic
tcagctgttaggaacctgaggctgg

SEQ ID NO: 54 - DNA, 22 bp, Artificial Sequence
  Synthetic
gtccttgaggagtacccaacag

SEQ ID NO: 55 - DNA, 23 bp, Artificial Sequence
  Synthetic
gtatgtgctgaggcctgatgatg

SEQ ID NO: 56 - DNA, 21 bp, Artificial Sequence
  Synthetic
ctctgctcagagtccatcctg

SEQ ID NO: 57 - DNA, 21 bp, Artificial Sequence
  Synthetic
ggttctacatttgggagctag

SEQ ID NO: 58 - DNA, 24 bp, Artificial Sequence
  Synthetic
cactggatgaatgaaaagccctgc

SEQ ID NO: 59 - DNA, 23 bp, Artificial Sequence
  Synthetic
gtaaacggatcacttggtaatcg

SEQ ID NO: 60 - DNA, 21 bp, Artificial Sequence
  Synthetic
gcggtttcagctattgggaac

SEQ ID NO: 61 - DNA, 24 bp, Artificial Sequence
  Synthetic
gtgatggccgtaatgcctggtacg

SEQ ID NO: 62 - DNA, 21 bp, Artificial Sequence
  Synthetic
tcagctattgggaacctgagg

SEQ ID NO: 63 - DNA, 21 bp, Artificial Sequence
  Synthetic
gaataagtccaggattggggc

SEQ ID NO: 64 - DNA, 21 bp, Artificial Sequence
  Synthetic
cacgagtcacaatcaacacgg

SEQ ID NO: 65 - DNA, 24 bp, Artificial Sequence
  Synthetic
gagtccttgaggagtacccaatag

SEQ ID NO: 66 - DNA, 26 bp, Artificial Sequence
  Synthetic
gaataaaatgtaccatcaggcaactc

SEQ ID NO: 67 - DNA, 24 bp, Artificial Sequence
  Synthetic
cactggatgaatgaaaagccctgc

SEQ ID NO: 68 - DNA, 23 bp, Artificial Sequence
  Synthetic
cgggttctgcatttaggagctag

SEQ ID NO: 69 - DNA, 5 bp, Artificial Sequence
  Synthetic
aaaaa

SEQ ID NO: 70 - DNA, 5 bp, Artificial Sequence
  Synthetic
ccccc

SEQ ID NO: 71 - RNA, 4 nt, Artificial Sequence
  Synthetic
uuuu

SEQ ID NO: 72 - DNA, 4 bp, Artificial Sequence
  Synthetic
tttt

(19) United States
(12) Patent Application Publication
(10) Pub. No.: US 2009/0176727 A1
(43) Pub. Date: Jul. 9, 2009

(54) DOUBLE-STRANDED RNA STRUCTURES AND CONSTRUCTS, AND METHODS FOR GENERATING AND USING THE SAME

(75) Inventors: Catherine J. PACHUK, Lansdale, PA (US); C. Satishchandran, Lansdale, PA (US); Daniel Edward McCallus, Oaks, PA (US)

Correspondence Address: COOLEY GODWARD KRONISH LLP;ATTN: Patent Group, Suite 1100, 777 - 6th Street, NW, WASHINGTON, DC, 20001, US

(73) Assignee: Nucleonics, Inc.

(21) Appl. No.: 12/247,770

(22) Filed: Oct. 8, 2008

Related U.S. Application Data
(63) Continuation of application No. 10/531,349, filed on Apr. 15, 2005, now abandoned.
(60) Provisional application No. 60/419,532, filed on Oct. 18, 2002.
(60) Provisional application No. 60/421,757, filed on Oct. 28, 2002.

(51) Int. Cl. A61K 31/7105; C12N 15/74; C12N 5/00; A61P 35/00

(52) U.S. Cl. 514/44; 536/23.1; 435/320.1; 435/375

(57) ABSTRACT

The present invention relates to novel double-stranded RNA (dsRNA) structures and dsRNA expression constructs, methods for generating them, and methods of utilizing them for silencing genes. Desirably, these methods specifically inhibit the expression of one or more target genes in a cell or animal (e.g., a mammal such as a human) without inducing toxicity. These methods can be used to prevent or treat a disease or infection by silencing a gene associated with the disease or infection. The invention also provides methods for identifying nucleic acid sequences that modulate a detectable phenotype, such as the function of a cell, the expression of a gene, or the biological activity of a target polypeptide.

### BACKGROUND OF THE INVENTION

In general, the invention relates to novel double-stranded RNA (dsRNA) structures and dsRNA expression constructs, methods for generating them, and methods of utilizing them for silencing genes. Desirably, these methods specifically inhibit the expression of one or more target genes in a eukaryotic cell, plant, or animal (e.g., a mammal, such as a human) without inducing toxicity.

Double-stranded RNA (dsRNA) has been shown to induce gene silencing in a number of different organisms. Gene silencing can occur through various mechanisms, one of which is post-transcriptional gene silencing (PTGS). In post-transcriptional gene silencing, transcription of the target locus is not affected, but the RNA half-life is decreased. Exogenous dsRNA has been shown to act as a potent inducer of PTGS in plants and animals, including nematodes, trypanosomes, and insects. Transcriptional gene silencing (TGS) is another mechanism by which gene expression can be regulated. In TGS, transcription of a gene is inhibited. The potential to harness dsRNA mediated gene silencing for research, therapeutic, and prophylactic indications is enormous. The exquisite sequence specificity of target mRNA degradation and the systemic properties associated with PTGS make this phenomenon ideal for functional genomics and drug development.

Some current methods for using dsRNA in vertebrate cells to silence genes result in undesirable non-specific cytotoxicity or cell death due to dsRNA-mediated stress responses, including the interferon response. A potential quagmire exists for the use of RNAi in vertebrate systems, including humans, because of the ability of dsRNA to trigger various toxicities in vertebrates, e.g., the type I interferon response as well as other RNA stress response pathways. Induction of a dsRNA-mediated stress response is rapid, and may result in cellular apoptosis or anti-proliferative effects. In addition to the potential for dsRNA to trigger toxicity in vertebrate cells, dsRNA gene silencing methods may result in non-specific or inefficient silencing.

Another hurdle facing the practical implementation of dsRNA-mediated gene silencing is the inefficient production and delivery of dsRNA structures, e.g., problems of inefficient production of dsRNAs from dsRNA expression constructs. Thus, improved methods are needed for specifically and efficiently silencing target genes without inducing toxicity or cell death, including methods for enhancing the formation of short interfering dsRNAs (siRNAs) in cells, tissues, and organs that lack or are deficient in Dicer and other enzymes which cleave long dsRNAs. Desirably, these methods may be used to inhibit gene expression in in vitro samples, cell culture, and in vivo in animals (e.g., vertebrates, such as mammals).

### SUMMARY OF THE INVENTION

One aspect of the invention includes dsRNA expression constructs which produce dsRNA molecules or dsRNA complexes with mismatched regions. Another aspect involves gene silencing using a dsRNA molecule or dsRNA complex that has one or more mismatched regions. The single-stranded, mismatched regions in the secondary structure of the dsRNA molecule or dsRNA complex are cleaved by endogenous or exogenous RNase enzymes expressed in a cell, tissue, or mammal, resulting in short dsRNA molecules (siRNA) that can silence genes. Such dsRNA expression constructs, dsRNA molecules, and methods are especially useful for enhancing the formation of short dsRNA molecules in cells, tissues, or organs that lack or express low levels of the enzyme Dicer and other similar enzymes which cleave dsRNA.

Double-Stranded Nucleic Acids and Nucleic Acids Encoding them

In one aspect, the invention features a substantially pure ribonucleic acid (RNA) complex comprising a first strand and a second strand that hybridize to each other under physiological conditions to form a double-stranded (ds) region, in which the double-stranded region comprises one or more mismatched regions that separate the double-stranded region into two or more double-stranded segments. The mismatched regions of the dsRNA complex are capable of cleavage by single-strand ribonucleases.

The invention also features a substantially pure ribonucleic acid (RNA) molecule that includes in 5′ to 3′ order, a first strand, a loop, and a second strand, in which the first and second strands hybridize to each other under physiological conditions and the loop connects the first strand to the second strand to form at least one RNA double-stranded region. The RNA molecule further includes one or more mismatched regions that separate the RNA double-stranded region into two or more double-stranded segments. The mismatched regions, which are in a single-stranded conformation, are susceptible to cleavage by single-stranded ribonucleases.

The invention also features a substantially pure ribonucleic acid (RNA) molecule that has in 5′ to 3′ order, a first strand and a second strand, in which the first and second strands hybridize to each other under physiological conditions to form a first double-stranded region, and in which the first and second strands are joined by a loop; the RNA molecule further contains a third strand and a fourth strand, in which the third and fourth strands hybridize to each other under physiological conditions to form a second double-stranded region; finally, the RNA molecule contains a fifth strand that joins the second and the third strands.

In an embodiment of the above features of the invention, the substantially pure ribonucleic acid (RNA) molecule or RNA complex contains at least one 5′ end that has a Bernie Moss (BM) hairpin that includes in 5′ to 3′ order, an A strand and a B strand, in which the A and B strands are capable of hybridizing under physiological conditions to form a double-stranded region. The B strand of the BM hairpin is then joined to the RNA molecule by a C strand. The presence of the BM hairpin stabilizes the RNA molecule or RNA complex, relative to an RNA molecule or RNA complex lacking the BM hairpin.

In an embodiment of the features of the invention, at least a portion of at least one double-stranded segment of the RNA molecule or RNA complex has substantial sequence identity to a target polynucleotide, which provides the double-stranded segment of the RNA molecule or RNA complex with the ability to target a polynucleotide sequence (e.g., all or a portion of a gene, a gene promoter, or all or a portion of a gene and its promoter) in a biological sample, cell, or organism for silencing by RNAi, relative to a biological sample, cell, or organism not exposed to the RNA molecule or RNA complex.

In another embodiment of the invention, the RNA complex or RNA molecule has at least one double-stranded region that has at least two mismatched regions that separate the double-stranded region into at least three double-stranded segments (each segment of which can have, e.g., substantial sequence identity to a target polynucleotide).

In another embodiment, one or more of the double-stranded regions of the RNA molecule has at least 18, more preferably 19 contiguous nucleotides with substantial sequence identity to a target polynucleotide (e.g., 19 to 27 or 19 to 30).

The invention also includes a dsRNA molecule or a population of dsRNA molecules that has two strands. The dsRNA has two or more double-stranded regions that are each separated by a mismatched region. All or a portion of at least one double-stranded region (e.g., 2, 3, 4, 5, 7, 8, 9, 10, 11, 12, 15, 18, 20, or more double-stranded regions) has substantial sequence identity to all or a region of a target nucleic acid sequence (e.g., all or a region of a gene, a gene promoter, or a portion of a gene and its promoter). Cleavage of the single-stranded regions of the dsRNA molecule by endogenous or exogenously added RNases (in vitro or in vivo) and/or portions of the dsRNA by, e.g., Dicer or Argonaut, results in formation of siRNA molecules (i.e., the short dsRNA molecules), which specifically inhibit the expression of a target gene associated with the target nucleic acid sequence. In desirable embodiments, the mismatched region includes at least one nucleotide in one strand of the dsRNA that is not involved in base-pairing (i.e., the nucleotide does not base-pair with other nucleotides in the same strand and does not base-pair with other nucleotides in the other strand). In some embodiments, the mismatched region includes at least two nucleotides (e.g., at least one nucleotide from each strand) of the dsRNA that are not involved in base-pairing. Desirably, the mismatched region includes 1 to 3 nucleotides, 4 to 10 nucleotides, or 11 to 100 nucleotides, inclusive, in one or both strands of the dsRNA. In other embodiments, the dsRNA molecule includes at least 2, 3, 4, 5, 7, 8, 9, 10, 11, 12, 15, 18, 20, or more double-stranded regions that are each separated by a mismatched region.

In another aspect, the invention features a dsRNA or a population of dsRNA molecules that have one strand (e.g., a hairpin). The dsRNA has two double-stranded regions that are separated by a mismatched region and has a loop. All or a portion of at least one double-stranded region (e.g., 2, 3, 4, 5, 7, 8, 9, 10, 11, 12, 15, 18, 20, or more double-stranded regions) has substantial sequence identity to a region of a target nucleic acid sequence (e.g., all or a region of a gene, a gene promoter, or a portion of a gene and its promoter) and specifically inhibits the expression of a target gene associated with the target nucleic acid sequence. In some embodiments, the mismatched region includes at least one nucleotide (e.g., 1 to 3 nucleotides, 4 to 10 nucleotides, or 11 to 100 nucleotides) in the dsRNA that is not involved in base-pairing (i.e., the nucleotide does not base-pair with either other nucleotides in the mismatched region and does not base-pair with other nucleotides in other regions of the dsRNA). Desirably, the dsRNA includes at least 2, 3, 4, 5, 7, 8, 9, 10, 11, 12, 15, 18, 20, or more double-stranded regions that are each separated by a mismatched region. In some embodiments, the mismatched regions are either all upstream from the loop (i.e., all in the 5′ region of the dsRNA before the loop) or are all downstream from the loop (i.e., all in the 3′ region of the dsRNA after the loop). In other embodiments, mismatched regions are present both upstream and downstream from the loop. In some embodiments, a mismatched region upstream from the loop is in the position corresponding to a mismatched region downstream from the loop in the hairpin structure (i.e., both mismatched regions are an equal distance from the loop.

In yet another aspect, the invention features a dsRNA or a population of dsRNA molecules that have one strand with two or more hairpin regions (e.g., a strand with 3, 4, 5, 6, 7, 8, 9, 10, 12, 14, or more hairpin regions). All or a portion of at least one double-stranded region (e.g., 2, 3, 4, 5, 7, 8, 9, 10, 11, 12, 15, 18, 20, or more double-stranded regions) within at least one hairpin region (e.g., 2, 3, 4, 5, 6, 7, 8, 9, 10, 12, 14, or more hairpins) has substantial sequence identity to a region of a target nucleic acid sequence (e.g., all or a region of a gene, a gene promoter, or a portion of a gene and its promoter) and specifically inhibits the expression of a target gene associated with the target nucleic acid sequence. Desirably, two or more hairpin regions are each separated by a spacer between each hairpin (e.g., a single-stranded region of between 1 to 100, 1 to 50, 1 to 25, 1 to 10, or 2 to 7 nucleotides). In desirable embodiments, the loop within one or more hairpin regions or the spacer between two hairpin regions is cleaved by an enzyme (e.g., an endogenous or exogenous RNase expressed in a cell in which gene silencing is desired). In desirable embodiments, one or more of the hairpin regions are shRNAs (short hairpin dsRNAs) with a double-stranded stem region of about 19 to 30, about 19 to 27, or about 19 to 23 basepairs in which all or a portion of at least one double-stranded region has substantial sequence identity to a target polynucleotide sequence (e.g., all or a region of a gene, a promoter, or a portion of a gene and its promoter).

In desirable embodiments of the above aspect, at least one hairpin region has two double-stranded regions that are separated by a mismatched region and has a loop. In some embodiments, the mismatched region includes at least one nucleotide in the dsRNA that is not involved in base-pairing (i.e., the nucleotide does not base-pair with other nucleotides in the mismatched region and does not base-pair with other nucleotides in other regions of the dsRNA). Desirably, the dsRNA includes at least 2, 3, 4, 5, 7, 8, 9, 10, 11, 12, 15, 18, 20, or more double-stranded regions that are each separated by a mismatched region. In some embodiments, the mismatched regions are either all upstream from the loop (i.e., all in the 5′ region of the dsRNA before the loop) or are all downstream from the loop (i.e., all in the 3′ region of the dsRNA after the loop). In other embodiments, mismatched regions are present both upstream and downstream from the loop. In some embodiments, a mismatched region upstream from the loop is in the position corresponding to a mismatched region downstream from the loop in the hairpin structure (i.e., both mismatched regions are an equal distance from the loop).

In a related aspect, the invention features a nucleic acid molecule (e.g., a deoxyribonucleic acid (DNA) molecule, such as a vector) that encodes one or more of the dsRNA molecules of any of the above aspects.

In yet another aspect, the invention features two or more nucleic acid molecules (e.g., DNA molecules, such as vectors) that encode one or more strands of a dsRNA molecule of any of the above aspects. In one embodiment, each DNA molecule encodes one strand of a dsRNA that forms a duplex of two strands.

### Desirable Double-Stranded RNA Molecules

In desirable embodiments of any of the above aspects, the dsRNA has at least 2, 3, 4, 5, 7, 8, 9, 10, 11, 12, 15, 18, 20, 50, 100 or more mismatched regions. Desirably, one or more mismatched regions or loops of the dsRNA (e.g., 2, 3, 4, 5, 7, 8, 9, 10, 11, 12, 15, 18, 20, 50, 100 or more mismatched regions or loops) are cleaved by an enzyme (e.g., an endogenous or exogenous RNase expressed in a cell, tissue, organ, or mammal in which gene silencing is desired). An exemplary RNase that may be added by co-expression is ribonuclease TI. In desirable embodiments, the amount of dsRNA with one or more mismatched regions that is cleaved in vitro or in vivo is at least 10, 20, 40, 60, 80, 100, 200, 300, or 500% more that the corresponding amount of a control dsRNA without one or more of the mismatched regions that is cleaved under the same conditions.

In other desirable embodiments of any of the above aspects, the dsRNA is a multiple epitope dsRNA that has two or more double-stranded regions (e.g., 2, 3, 4, 5, 6, 8, 10, 15, or more ds regions), in which all or a portion of at least two of the double-stranded regions have substantial identity to all or a region of a target nucleic acid sequence (e.g., all or a region of a gene, a gene promoter, or a portion of a gene and its promoter; e.g., 2, 3, 4, 5, 6, 8, 10, 15, or more target genes). For example, the double-stranded regions can have substantial sequence identity to the same target gene or the same region of a target gene, different regions of the same target gene, different target genes, or different regions of different target genes. Desirably, following cleavage of the multiple epitope RNA molecule to liberate the dsRNA regions (i.e., the siRNA molecules), the siRNA molecules specifically silence one or more of the target genes to which they are directed. In various embodiments, the double-stranded region is at least 19, 20, 21, 22, 23, 24, 25, 26, 27, or 30 nucleotides in length or even at least 30, 40, 50, 100, or 200 nucleotides in length, inclusive. In particular embodiments, the double-stranded region is 19 to 100, 19 to 75, 19 to 50, 19 to 30, or 19 to 25 nucleotides in length, inclusive. Desirably, the double-stranded region has at least 19, 20, 21, 22, 23, 24, 25, or 26 contiguous nucleotides or even at least 30, 40, 50, or 100 contiguous nucleotides that are all in a double-stranded conformation and all or a portion of the nucleotides in the double-stranded region have 100% sequence identity to a region of a target nucleic acid sequence (e.g., all or a region of a gene, a gene promoter, or a portion of a gene and its promoter). The double-stranded region may or may not have other nucleotides (i.e., nucleotides outside of this region of 100% identity to the target nucleic acid sequence) that are not in a double-stranded confirmation (i.e., nucleotides not base-paired with other nucleotides in the double-stranded region). In some embodiments, such a dsRNA with a less than 100% complementary double-stranded region participates in a micro interference (miRNA) pathway. Double-stranded RNA molecules have an overall length of between 40 and 20,000 nucleotides; desirably 40 and 10,000 nucleotides; more desirably 40 and 5,000 nucleotides; and most desirably 100 and 1000 nucleotides, inclusive. In some embodiments, the dsRNA has a dumbbell or cloverleaf structure, or is an “udderly” structured dsRNA having multiple stem-loop structures separated by single-stranded spacer regions. In other embodiments, the dsRNA has multiple stem-loop structures separated by double-stranded regions. Some such structures comprise one or more sets of paired stem-loop or hairpin structures which are 180 degrees opposed to each other, including such structures wherein three hairpin dsRNAs assume a cloverleaf configuration.

### Methods for Generating Double-Stranded Nucleic Acid Molecules

In another aspect, the invention features a method of generating one or more dsRNA molecules of any of the above aspects. This method involves administering one or more nucleic acid molecules (e.g., a DNA molecule, such as a vector) encoding a dsRNA molecule of any of the above aspects to an in vitro sample, cell, or mammal under conditions that allow transcription of the dsRNA molecule. In some embodiments, the nucleic acid molecule encoding the duplex dsRNA has one strand of the dsRNA molecule under the control of the one promoter and the second strand of the dsRNA molecule under the control of a different promoter. Alternatively, both strands of the dsRNA molecule can be under the control of the same promoter in the nucleic acid molecule. The two strands may be encoded by the same vector or different vectors. In particular embodiments, the method involves synthesizing the sense strand and the antisense strand of a duplex dsRNA from separate cistrons (transcription units). In other embodiments, the method involves synthesizing a nucleic acid molecule encoding a dsRNA of the invention by ligating one or more nucleic acid fragments to form the nucleic acid molecule. In particular embodiments, the nucleic acid fragments encode different hairpin regions with or without a spacer. In some embodiments, the nucleic acid molecule encoding the dsRNA will include 5′ and/or 3′ flanking regions, including 5′ transcription initiation regions and/or 5′ stabilizing hairpin regions, and/or 3′ spacer/terminator regions.

### Co-Expression of Dicer Enzyme

In desirable embodiments of any of the above aspects of the invention, exogenous dicer (e.g., mouse or human dicer or dicer that is not from a nematode such as C. elegans) is expressed in a cell, tissue, or animal (e.g., a mammal, such as a human). In some embodiments, endogenous dicer (e.g., mouse or human dicer or dicer that is not from a nematode, such as C. elegans) is over-expressed under the control of a heterologous promoter in a cell, tissue, or animal (e.g., a mammal such as a human). Desirably, this expression of dicer increases the cleavage of a dsRNA of the invention and/or the silencing of a target gene by at least 25, 50, 100, 200, 500, 750, or 100%.

### Pharmaceutical Compositions

The invention also features a pharmaceutical composition that includes one or more dsRNA molecules or nucleic acid molecules encoding dsRNA molecules (e.g., partial or full hairpins) in an acceptable vehicle. In one such aspect, the invention features a pharmaceutical composition that includes one or more nucleic acid molecules of any of the aspects of the invention in an acceptable vehicle.

In another aspect, the invention provides a pharmaceutical composition which includes at least one short dsRNA (e.g., 1, 2, 3, 5, 8, 10, 20, 30, or more different short dsRNA species) and at least one long dsRNA (e.g., 1, 2, 3, 5, 8, 10, 20, 30, or more different long dsRNA species) in an acceptable vehicle (e.g., a pharmaceutically acceptable carrier).

In various embodiments, the pharmaceutical composition includes about 1 ng to about 20 mg of nucleic acid, e.g., RNA, DNA, plasmids, viral vectors, recombinant viruses, or mixtures thereof, which provide the desired amounts of the respective dsRNA molecules (dsRNA homologous to a target nucleic acid and/or dsRNA to inhibit toxicity). In some embodiments, the composition contains about 10 ng to about 10 mg of nucleic acid, about 0.1 mg to about 500 mg, about 1 mg to about 350 mg, about 25 mg to about 250 mg, or about 100 mg of nucleic acid. If desired, the dosage regimen of the short dsRNA may be adjusted to achieve the optimal inhibition of the dsRNA-activated protein kinase (PKR) and/or other dsRNA-mediated stress responses, and the dosage regimen of the other dsRNA (e.g, long dsRNA) may be adjusted to optimize the desired sequence-specific silencing. Accordingly, a composition of the invention may contain different amounts of the two dsRNA molecules. Those of skill in the art of clinical pharmacology can readily arrive at such dosing schedules using routine experimentation.

Suitable carriers include, but are not limited to, saline, buffered saline, dextrose, water, glycerol, ethanol, and combinations thereof. The composition can be adapted for the mode of administration and can be in the form of, for example, a pill, tablet, capsule, spray, powder, or liquid. In some embodiments, the pharmaceutical composition contains one or more pharmaceutically acceptable additives suitable for the selected route and mode of administration. These compositions may be administered by, without limitation, any parenteral route including intravenous, intra-arterial, intramuscular, subcutaneous, intradermal, intraperitoneal, intrathecal, as well as topically, orally, and by mucosal routes of delivery such as intranasal, inhalation, rectal, vaginal, buccal, and sublingual. In some embodiments, the pharmaceutical compositions of the invention are prepared for administration to vertebrate (e.g., mammalian) subjects in the form of liquids, including sterile, non-pyrogenic liquids for injection, emulsions, powders, aerosols, tablets, capsules, enteric coated tablets, or suppositories.

Kits for Synthesis or Administration of dsRNA Molecules

In a related aspect, the invention provides a kit for generation of a dsRNA molecule of the invention.

Cells with Nucleic Acids of the Invention

The invention also features cells with one or more of the nucleic acid molecules of the invention. In one such aspect, the invention features a cell or a population of cells that expresses a dsRNA molecule that modulates a detectable phenotype, including, without limitation, a dsRNA that: (i) modulates a function of the cell, (ii) modulates the expression of a target gene (e.g., an endogenous gene or gene of a pathogen) in the cell, and/or (iii) modulates the biological activity of a target protein (e.g., an endogenous protein or protein of a pathogen) in the cell. In some embodiments, this dsRNA molecule has mismatched regions or one strand with two or more hairpin regions separated by single-stranded regions and/or double-stranded regions generated in vivo from a dsRNA expression construct. Optionally, the dsRNA expression construct includes 5′ and/or 3′ flanking regions to promote the desired initiation and/or termination of transcription, and/or 5′ stability-promoting hairpin region.

In other embodiments, the dsRNA is encoded by a vector that has an origin of replication that permits replication of the vector in the cell. Desirably, the vector is maintained in the cell or in the progeny of the cell after 1, 5, 10, 15, 30, 50, 100, or more cell divisions.

In desirable embodiments, the cell or population of cells also has one or more dsRNA molecules (e.g., 1, 2, 3, 5, 8, 10, 20, 30, or more different dsRNA species) that desirably inhibit an interferon response or a dsRNA-mediated stress response. In some embodiments, the cell contains only one or more dsRNA molecules that inhibit a target gene or only a dsRNA expression construct encoding the one or more dsRNA molecules (e.g., a stably integrated vector). Desirably, the cell or population of cells are administered the dsRNA molecules or dsRNA expression vector by one or more methods of the invention (see below). In other embodiments, the one or more dsRNA molecules are administered with or contain specific dsRNA regions that inhibit or prevent an interferon response or a dsRNA stress response. These specific dsRNA regions are typically short non-specific dsRNA regions that are not targeted to a specific nucleic acid sequence (i.e., these short dsRNA molecules do not contain a region of substantial sequence identity to a target nucleic acid sequence (e.g., all or a region of a gene, a gene promoter, or a portion of a gene and its promoter) as do the dsRNA molecules of the present invention).

### Methods for Inhibiting Gene Expression in Cells or Animals

The invention also features novel methods for silencing genes that produce few, if any, toxic side-effects. In particular, these methods involve administering to a cell or animal an agent that provides one or more dsRNA molecules that have one or more double-stranded regions (preferably two or more double-stranded regions), in which all or a portion of at least one double-stranded region has substantial sequence identity to a region of a target nucleic acid sequence (e.g., all or a region of a gene, a gene promoter, or a portion of a gene and its promoter) and that, following cleavage of the dsRNA molecule as is discussed herein, specifically inhibit the expression of a target gene associated with the target nucleic acid molecule. If desired, an agent that provides one or more short non-specific dsRNA molecules, which differ from the dsRNA having substantial identity to a target nucleic acid sequence, can also be administered to inhibit possible toxic effects or non-specific gene silencing that may otherwise be induced by the dsRNA molecules of the present invention. In some embodiments, the agent is a nucleic acid molecule (e.g., a DNA molecule, such as a vector) or a pharmaceutical composition of any of the above aspects.

Accordingly, in one such aspect, the invention features a method for reducing or inhibiting the expression of a target gene in a cell (e.g., a eukaryotic cell, a plant cell, an animal cell, an invertebrate cell, a vertebrate cell, such as a mammalian or human cell, or a pathogen cell). This method involves introducing into the cell a first agent that provides to the cell a first dsRNA molecule having one or more double-stranded regions (preferably two or more double-stranded regions), in which all or a portion of at least one double-stranded region has substantial sequence identity to a region of a target nucleic acid sequence associated with the gene (e.g., all or a region of the gene sequence, a sequence of the promoter of the gene, or a portion of the gene and its promoter) and specifically inhibits the expression of the target gene. Exemplary pathogens include bacteria, yeast, and fungus. In some embodiments, the first dsRNA inhibits the expression of an endogenous gene in a vertebrate cell or a pathogen cell (e.g., a bacterial, a yeast cell, or a fungal cell), or inhibits the expression of a pathogen gene in a cell infected with the pathogen (e.g., a plant or animal cell).

In some embodiments of the above aspect, a second agent that provides to the cell a second, non-specific dsRNA molecule is also introduced into the cell. This second dsRNA differs from the first dsRNA in that it does not have substantial sequence identity to a target nucleic acid sequence (e.g., all or a region of a gene, a gene promoter, or a portion of a gene and its promoter). Administration of the second dsRNA reduces or inhibits the interferon response or dsRNA-mediated toxicity associated with administration of the first dsRNA molecule. In some embodiments, the second dsRNA binds PKR and inhibits the dimerization and/or activation of PKR. In some embodiments of the above aspects, the second, non-specific dsRNA and/or the first dsRNA is a dsRNA molecule with mismatched regions or one strand with two or more hairpin regions separated by single-stranded regions, as described herein.

In another aspect, the invention provides a method for reducing or inhibiting the expression of a target gene in an animal (e.g., an invertebrate or a vertebrate, such as a mammal, e.g., a human). This method involves introducing into the animal a first agent that provides to the animal a first dsRNA molecule. The first dsRNA molecule has one or more double-stranded regions (preferably two or more double-stranded regions), in which all or a portion of at least one double-stranded region has substantial sequence identity to a region of a target nucleic acid sequence associated with the target gene (e.g., all or a region of the gene sequence, a sequence of the promoter of the gene, or a portion of the gene and its promoter) and, when the single-stranded regions of the dsRNA molecule are cleaved by endogenous or exogenous single-strand ribonucleases, as discussed herein, which liberate the dsRNA regions of the dsRNA molecule (i.e., the siRNA molecules), the result is a reduction or inhibition of expression of the target gene. In some embodiments, the first dsRNA inhibits the expression of an endogenous gene in an animal, or, alternatively, the dsRNA inhibits the expression of a gene of a pathogen (e.g., a bacteria, a yeast, a fungus, a protozoan, a parasite, or a virus) that has infected an animal.

In some embodiments of the above aspect, a second agent that provides to the cell a second, non-specific dsRNA molecule is also introduced into the cell. This second dsRNA differs from the first dsRNA in that it does not have substantial sequence identity to a target nucleic acid sequence (e.g., all or a region of a gene, a gene promoter, or a portion of a gene and its promoter). Administration of the second dsRNA reduces or inhibits the interferon response or dsRNA-mediated toxicity associated with administration of the first dsRNA molecule. In some embodiments, the second dsRNA binds PKR and inhibits the dimerization and/or activation of PKR. In some embodiments of the above aspects, the second, non-specific dsRNA and/or the first dsRNA is a dsRNA molecule with mismatched regions or one strand with two or more hairpin regions separated by single-stranded regions, as described herein.

### Methods for Treating or Preventing Disease in an Animal by Inhibiting Gene Expression

In yet another aspect, the invention provides a method for treating, stabilizing, or preventing a disease or disorder in an animal (e.g., an invertebrate or a vertebrate such as a mammal or human). This method involves introducing into the animal a first agent that provides to the animal a first dsRNA molecule. The first dsRNA molecule has one or more double-stranded regions (preferably two or more double-stranded regions), in which all or a portion of at least one double-stranded region has substantial sequence identity to a region of a target nucleic acid sequence (e.g., all or a region of a gene, a gene promoter, or a portion of a gene and its promoter) and specifically reduces or inhibits the expression of a target gene associated with the disease or disorder, following cleavage of the first dsRNA molecule to liberate the dsRNA regions within the first dsRNA molecule (i.e., the siRNA molecules), as is discussed herein. In some embodiments, the target gene is a gene associated with cancer, such as an oncogene, or a gene encoding a protein associated with a disease, such as a mutant protein, a dominant negative protein, or an overexpressed protein.

In some embodiments of the above aspect, a second agent that provides to the cell a second, non-specific dsRNA molecule is also introduced into the cell. This second dsRNA differs from the first dsRNA in that it does not have substantial sequence identity to a target nucleic acid sequence (e.g., all or a region of a gene, a gene promoter, or a portion of a gene and its promoter). Administration of the second dsRNA reduces or inhibits the interferon response or dsRNA-mediated toxicity associated with administration of the first dsRNA molecule. In some embodiments, the second dsRNA binds PKR and inhibits the dimerization and/or activation of PKR. In some embodiments of the above aspects, the second, non-specific dsRNA and/or the first dsRNA is a dsRNA molecule with mismatched regions or one strand with two or more hairpin regions separated by single-stranded regions, as described herein.

Exemplary cancers that can be treated, stabilized, or prevented using the above methods include prostate cancers, breast cancers, ovarian cancers, pancreatic cancers, gastric cancers, bladder cancers, salivary gland carcinomas, gastrointestinal cancers, lung cancers, colon cancers, melanomas, brain tumors, leukemias, lymphomas, and carcinomas. Benign tumors may also be treated or prevented using the methods of the present invention. Other cancers and cancer related genes that may be targeted are disclosed in, for example, WO 00/63364, WO 00/44914, and WO 99/32619.

Exemplary endogenous proteins that may be associated with disease include ANA (anti-nuclear antibody) found in SLE (systemic lupus erythematosis), abnormal immunoglobulins including IgG and IgA, Bence Jones protein associated with various multiple myelomas, and abnormal amyloid proteins in various amyloidoses including hereditary amyloidosis and Alzheimer's disease. In Huntington's Disease, a genetic abnormality in the HD (huntingtin) gene results in an expanded tract of repeated glutamine residues. In addition to this mutant gene, HD patients have a copy of chromosome 4 which has a normal sized CAG repeat. Thus, methods of the invention can be used to silence the abnormal gene, but not the normal gene. In various embodiments, a gene encoding a disease-causing protein is silenced using the dsRNA molecules of the invention, in which the dsRNA molecules have one or more double-stranded regions (preferably two or more double-stranded regions), in which all or a portion of at least one double-stranded region has substantial sequence identity to, e.g., all or a region of the gene sequence encoding the disease-causing protein, a sequence of the promoter of the gene encoding the disease-causing protein, or a portion of the gene encoding the disease-causing protein and its promoter. In other embodiments, a second, non-specific dsRNA that does not have substantial sequence identity to a target nucleic acid sequence (e.g., a gene encoding a disease-causing protein, or its promoter) is also administered to the cell, thereby reducing or inhibiting the dsRNA stress response that might otherwise be associated with administration of the dsRNA molecules of the invention (i.e., those having regions of dsRNA with substantial sequence identity to a target nucleic acid sequence, e.g., a target gene).

### Methods for Treating or Preventing Infection in an Animal by Inhibiting Gene Expression

In still another aspect, the invention features a method for treating, stabilizing, or preventing an infection in an animal (e.g., an invertebrate or a vertebrate, such as a mammal, e.g., a human). This method involves introducing into the animal a first agent that provides to the animal a first dsRNA. The first dsRNA molecule has one or more regions that are double-stranded (preferably two or more double-stranded regions), in which all or a portion of at least one double-stranded region has substantial sequence identity to a target nucleic acid sequence (e.g., all or a region of a gene, a gene promoter, or a portion of a gene and its promoter) in an infectious pathogen (e.g., a virus, a bacterium, a yeast, a fungus, a protozoan, or a parasite) or in a cell infected with the pathogen. Following administration of the dsRNA molecule and its cleavage by endogenous or exogenously provided single-stranded RNases to liberate the dsRNA regions within the first dsRNA molecule (i.e., the siRNA molecules), as is discussed herein, the dsRNA molecule specifically reduces or inhibits the expression of a target gene in a cell of the pathogen or a cell infected with the pathogen. In various embodiments, the pathogen is an intracellular or extracellular pathogen. In some embodiments, the target nucleic acid sequence is a gene of the pathogen that is necessary for replication and/or pathogenesis, or a gene encoding a cellular receptor necessary for a cell to be infected with the pathogen.

In some embodiments of the above aspect, a second agent that provides to the cell a second, non-specific dsRNA molecule is also introduced into the cell. This second dsRNA differs from the first dsRNA in that it does not have substantial sequence identity to a target nucleic acid sequence (e.g., all or a region of a gene, a gene promoter, or a portion of a gene and its promoter). Administration of the second dsRNA reduces or inhibits the interferon response or dsRNA-mediated toxicity associated with administration of the first dsRNA molecule. In some embodiments, the second dsRNA binds PKR and inhibits the dimerization and/or activation of PKR. In some embodiments of the above aspects, the second, non-specific dsRNA and/or the first dsRNA is a dsRNA molecule with mismatched regions or one strand with two or more hairpin regions separated by single-stranded regions, as described herein.

In further embodiments of any of the above aspects, the methods of administering a dsRNA molecule or a nucleic acid molecule encoding the dsRNA molecule (e.g., a DNA molecule, such as a vector; referred to herein as a dsRNA expression construct or a dsRNA expression vector) includes contacting an in-dwelling device with an agent comprising the dsRNA molecule or dsRNA expression vector prior to, concurrent with, or following the administration of the in-dwelling device to a patient. In-dwelling devices include, but are not limited to, surgical implants, prosthetic devices, and catheters, i.e., devices that are introduced to the body of an individual and remain in position for an extended time. Such devices include, for example, artificial joints, heart valves, pacemakers, vascular grafts, vascular catheters, cerebrospinal fluid shunts, urinary catheters, and continuous ambulatory peritoneal dialysis (CAPD) catheters. Desirably, the dsRNA molecule prevents the growth of bacteria on the device. In some embodiments, the first dsRNA molecule inhibits the expression of a bacterial gene in a bacterium, a cell infected with a bacterium, or an animal infected with a bacterium.

In other desirable embodiments, the bacterial infection is due to one or more of the following bacteria: Chlamydophila pneumoniae, C. psittaci, C. abortus, Chlamydia trachomatis, Simkania negevensis, Parachlarnydia acanthamoebae, Pseudomonas aeruginosa, P. alcaligenes, P. chlororaphis, P. fluorescens, P. luteola, P. mendocina, P. monteilii, P. oryzihabitans, P. pertocinogena, P. pseudalcaligenes, P. putida, P. stutzeri, Burkholderia cepacia, Aeromonas hydrophilia, Escherichia coli, Citrobacter freundii, Salmonella typhimuriunm, S. typhi, S. paratyphi, S. enteritidis, Shigella dysenteriae, S. flexneri, S. sonnei, Enterobacter cloacae, E. aerogenes, Klebsiella pneumoniae, K. oxytoca, Serratia marcescens, Francisella tularensis, Morganella morganii, Proteus mirabilis, Proteus vulgaris, Providencia alcalifaciens, P. rettgeri, P. stuartii, Acinetobacter calcoaceticus, A. haemolyticus, Yersinia enterocolitica, Y. pestis, Y. pseudotuberculosis, Y. intermedia, Bordetella pertussis, B. parapertussis, B. bronchiseptica, Haemophilus influenzae, H. parainfluenzae, H. haemolyticus, H. parahaemolyticus, H. ducreyi, Pasteurella multocida, P. haemolytica, Branhamella catarrhalis, Helicobacterpylori, Campylobacterfetus, C. jejuni, C. coli, Borrelia burgdorferi, V. cholerae, V. parahaemolyticus, Legionella pneumophila, Listeria monocytogenes, Neisseria gonorrhea, N. meningitidis, Kingella denttrificans, K. kingae, K. oralis, Moraxella catarrhalis, M. atlantae, M. lacunata, M. nonliquefaciens, M. osloensis, M. phenylpyruvica, Gardnerella vaginalis, Bacteroides fragilis, Bacteroides distasonis, Bacteroides 3452A homology group, Bacteroides vulgatus, B. ovalus, B. thetaiotaomicron, B. uniformis, B. eggerthii, B. splanchniicus, Clostridium difficile, Mycobacterium tuberculosis, M. avium, M intracellulare, M. leprae, C. diphtheriae, C. ulcerans, C. accolens, C. afermentans, C. amycolatum, C. argentorense, C. auris, C. bovis, C. confusunm, C. coyleae, C. durum, C. falsenii, C. glucuronolyticum, C. imitaiis, C. jeikeiuin, C. kutscheri, C. kroppenstedtii, C. lipophilum, C. macginleyi, C. matruchoti, C. mucifaciens, C. pilosum, C. propinquum, C. renale, C. riegelii, C. sanguinis, C. singulare, C. striatum, C. sundsvallense, C. thomssenii, C. urealyticum, C. xerosis, Streptococcus pneumoniae, S. agalactiae, S. pyogenzes, Enterococcus avium, E. casseliflavus, E. cecorum, E. dispar, E. durans, E. faecalis, E. faecium, E. flavescens, E. gallinarum, E. hirae, E. malodoratus, E. mundtii, E. pseudoavium, E. raffinosus, E. solitarius, Staphylococcus aureus, S. epidermidis, S. saprophyticus, S. intermedius, S. hyicus, S. haemolyticus, S. hominis, and/or S. saccharolyticus.

Preferably, the dsRNA molecule is administered in an amount sufficient to prevent, stabilize, or inhibit the growth of the pathogen or to kill the pathogen.

In some embodiments, the first dsRNA molecule inhibits the expression of a yeast gene in a yeast cell, a cell infected with yeast, or an animal infected with yeast.

In some embodiments, the first dsRNA molecule inhibits the expression of a viral gene in a cell infected with a virus, or in an animal infected with virus. In desirable embodiments, the viral infection relevant to the methods of the invention is an infection by one or more of the following viruses: Hepatitis B, Hepatitis C, picornavirus, polio, HIV, coxsacchie, herpes simplex virus Type I and 2, St. Louis encephalitis, Epstein-Barr, myxoviruses, JC, coxsakieviruses B, togaviruses, measles, paramyxoviruses, echoviruses, bunyaviruses, cytomegaloviruses, varicella-zoster, mumps, equine encephalitis, lymphocytic choriomeningitis, rhabodoviruses including rabies, simian virus 40, human polyoma virus, parvoviruses, papilloma viruses, primate adenoviruses, coronaviruses, retroviruses, Dengue, yellow fever, Japanese encephalitis virus, and/or BK. In some embodiments, the first dsRNA molecule inhibits the expression of a viral gene in a cell or animal infected with a virus.

Particularly suitable for the therapeutic and prophylactic methods of the invention are DNA viruses or viruses that have an intermediary DNA stages. Among such viruses are included, without limitation, viruses of the species Retrovirus, Herpesvirus, Hepadenovirus, Poxvirus, Parvovirus, Papillomavirus, and Papovavirus. Specifically some of the more desirable viruses to treat with this method include, without limitation, HIV, BBV, HSV, CMV, HPV, HTLV and EBV. The agent used in this method provides to the cell of the mammal an at least partially double-stranded RNA molecule as described herein, which includes one or more double-stranded regions (preferably two or more double-stranded regions), in which all or a portion of at least one double-stranded region has substantial sequence identity to a target nucleic acid sequence of a virus (e.g., all or a region of a viral gene, a viral gene promoter, or a portion of a viral gene and its promoter). In an embodiment of this method of the invention, the viral nucleic acid sequence is necessary for replication and/or pathogenesis of the virus in an infected mammalian cell. Such viral target genes are necessary for the propagation of the virus and include, e.g., the HIV gag, env, and pol genes, the HPV6 LI and E2 genes, the HPV II LI and E2 genes, the HPV 16 E6 and E7 genes, the BPV 18 E6 and E7 genes, the HBV surface antigens, the HBV core antigen, HBV reverse transcriptase, the HSV gD gene, the HSVvp 16 gene, the HSV gC, gH, gL and gB genes, the HSV ICPO, ICP4 and ICP6 genes, Varicella zoster gB, gC and gH genes, and the BCR-abl chromosomal sequences, and non-coding viral polynucleotide sequences which provide regulatory functions necessary for transfer of the infection from cell to cell, e.g., the HIV LTR, and other viral promoter sequences, such as HSV vp 16 promoter, HSV-ICPO promoter, HSV-ICP4, ICP6 and gD promoters, the HBV surface antigen promoter, the HBV pre-genomic promoter, among others.

Thus, this method can be used to treat mammalian subjects already infected with a virus, such as HIV, in order to shut down or inhibit a viral gene function essential to virus replication and/or pathogenesis, such as HIV gag. Alternatively, this method can be employed to inhibit the functions of viruses which exist in mammals as latent viruses, e.g., Varicella zoster virus, and are the causative agents of the disease known as shingles. Similarly, diseases such as atherosclerosis, ulcers, chronic fatigue syndrome, and autoimmune disorders, recurrences of HSV-I and HSV-2, HPV persistent infection, e.g., genital warts, and chronic BBV infection among others, which have been shown to be caused, at least in part, by viruses, bacteria, or another pathogen, can be treated according to this method by targeting certain viral polynucleotide sequences essential to viral replication and/or pathogenesis in the mammalian subject.

Still another analogous embodiment of the above “anti-viral” methods of the invention includes a method for treatment or prophylaxis of a virally induced cancer in a mammal. Such cancers include HPV E6/E7 virus-induced cervical carcinoma, HTLV-induced cancer, and EBV induced cancers, such as Burkitts lymphoma, among others. This method is accomplished by administering to the mammal a composition, as described herein, in which the target polynucleotide is a sequence encoding a tumor antigen or functional fragment thereof, or a non-expressed regulatory sequence, which antigen or sequence function is required for the maintenance of the tumor in the mammal. Among such sequences are included, without limitation, HPV16 E6 and E7 sequences and HPV 18 E6 and E7 sequences. Others may readily be selected by one of skill in the art. The composition is administered in an amount effective to reduce or inhibit the function of the antigen in the mammal, and preferably employs the composition components, dosages, and routes of administration as described herein.

### Methods for Treating or Preventing an Immune Response in an Animal by Inhibiting Gene Expression

In another aspect, the invention features a method for reducing or preventing an immune response in an animal (e.g., a mammal, such as a human) to a transplanted cell, tissue, or organ. The method involves administering to the transplanted cell, tissue, or organ or to the animal receiving the cell, tissue, or organ a first agent that provides a first dsRNA molecule. The first dsRNA molecule attenuates the expression of a target nucleic acid sequence (e.g., all or a region of a gene associated with causing an immune response, a promoter of that gene, or a portion of both the gene and its promoter) in the transplanted cell, tissue, or organ or in the animal receiving the cell, tissue, or organ that can elicit an immune response in the recipient.

In some embodiments of the above aspect, a second agent is administered to the transplanted cell, tissue, or organ or to the animal receiving the cell, tissue, or organ that provides a second, non-specific dsRNA molecule. This second dsRNA differs from the first dsRNA molecule in that it does not have substantial sequence identity to a target nucleic acid sequence (e.g., all or a region of a gene, a gene promoter, or a portion of a gene and its promoter). Administration of the second dsRNA reduces or inhibits the interferon response or dsRNA-mediated toxicity associated with administration of the first dsRNA molecule. In some embodiments, the second dsRNA binds PKR and inhibits the dimerization and/or activation of PKR. In some embodiments of the above aspects, the second, non-specific dsRNA and/or the first dsRNA is a dsRNA molecule with mismatched regions or one strand with two or more hairpin regions separated by single-stranded regions, as described herein. See, e.g., the teaching of U.S. Ser. No. 60/375,636, filed Apr. 26, 2002 and U.S. Ser. No. 10/425,006 filed Apr. 28, 2003, “Methods of Silencing Genes Without Inducing Toxicity”, C. Pachuk, incorporated herein by reference.

Effect of the dsRNA Molecule Upon Administration to a Cell, Tissue, Organ, or Animal

In desirable embodiments of any of the above aspects, the first dsRNA molecule reduces or inhibits expression of a target gene by at least 20, 40, 60, 80, 90, 95, or 100%. In some embodiments, the first dsRNA molecule has multiple double-stranded regions, in which all or a portion of each double-stranded region has substantial sequence identity to a different nucleic acid sequence (e.g., all or a portion of a different gene, a different gene promoter, or all or a portion of a different gene and its promoter), and is administered to the cell or animal to inhibit the expression of multiple target genes. In other embodiments, a multiple epitope first dsRNA molecule that has double-stranded regions with substantial sequence identity to different target genes is administered to silence multiple target genes. For example, multiple oncogenes or multiple pathogen genes may be simultaneously silenced.

In various embodiments of any of the above aspects, the first agent (comprising the first dsRNA molecule) and/or a second agent (comprising the second, non-specific dsRNA) is a DNA molecule or DNA vector encoding the first and/or second dsRNA molecules (i.e., dsRNA expression vectors). In other embodiments, the first agent and/or the second agent is a dsRNA molecule, a single-stranded RNA molecule that assumes a double-stranded conformation inside the cell or animal (e.g., a multiple hairpin or “udderly” structured RNA, or a partial or full hairpin), or a combination of two single-stranded RNA molecules that are administered simultaneously or sequentially and that assume a double-stranded conformation inside the cell or animal. The first agent may be administered before, during, or after the administration of the second agent. In some embodiments, the first and second agents are expressed from the same or different nucleic acid molecules (e.g., the same vector encodes both the first and the second dsRNA molecules, different vectors encode the first and the second dsRNA molecules, or a different vector encodes one strand of the first and second dsRNA molecules, while a second vector encodes the second strand of the first and second dsRNA molecules). In various embodiments, the first agent provides a short dsRNA or a long dsRNA to the cell or animal. In some embodiments of the above aspects, the second, non-specific dsRNA and/or the first dsRNA is a dsRNA molecule with mismatched regions or one strand with two or more hairpin regions separated by single-stranded regions, as described herein.

In some embodiments, a cytokine is also administered to the cell or animal. Exemplary cytokines are disclosed in WO 00/63364, filed Apr. 19, 2000. In some embodiments, the expression of the target gene is increased to promote the amplification of the dsRNA molecule, resulting in more dsRNA molecules to silence the target gene. For example, a vector containing the target nucleic acid can be administered to the cell or animal before, during, or after the administration of the first and/or second agent.

Methods for Identifying Nucleic Acid Sequences of Interest by Transfecting Cells with a dsRNA Expression Library

The invention also features high throughput methods of using dsRNA-mediated gene silencing to identify a nucleic acid sequence associated with a detectable phenotype in a cell, e.g., a gene that modulates the function of a cell, that modulates expression of a target gene, or that modulates the biological activity of a target polypeptide, for example a target polypeptide, e.g., those polypeptides described herein. The method involves the use of specially constructed cDNA libraries derived from a cell (for example, a primary cell or a cell line that has an observable phenotype or biological activity e.g., an activity mediated by a target polypeptide or altered gene expression) that are transfected into cells to inhibit gene expression. The inhibition of gene expression by the present methods alters a detectable phenotype, e.g., the function of a cell, expression of a target gene, or the biological activity of a target polypeptide, and allows the nucleic acid sequence responsible for the alteration or modulation to be readily identified. The method may also utilize genomic libraries. While less desirable, the method may also utilize randomized nucleic acid sequences or a given sequence for which the function is not known, as described in, e.g., U.S. Pat. No. 5,639,595, the teaching of which is hereby incorporated by reference.

Accordingly, in one aspect, the invention features a method for identifying a nucleic acid sequence associated with a modulation of a detectable phenotype in a cell, (e.g., a gene that modulates the function of a cell, that modulates expression of a target gene in a cell, or that modulates the biological activity of a target polypeptide in a cell.) The method involves (a) transforming a population of cells with a dsRNA expression library, where at least two cells of the population of cells are each transformed with a different nucleic acid sequence from the dsRNA expression library, and where at least one encoded dsRNA molecule specifically reduces or inhibits the expression of a target gene in at least one cell; (b) optionally selecting for a cell in which the gene is expressed in the cell; and (c) assaying for a modulation of a detectable phenotype of the cell, wherein detection of said modulation identifies a nucleic acid sequence associated with the detectable phenotype of the cell (e.g., a specific target gene associated with the detectable phenotype of the cell). In a desirable embodiment, assaying for a modulation in the function of a cell involves measuring cell motility, apoptosis, cell growth, cell invasion, vascularization, cell cycle events, cell differentiation, cell dedifferentiation, neuronal cell regeneration, or the ability of a cell to support viral replication.

If desired, a second, non-specific dsRNA molecule, or a nucleic acid molecule (e.g., a vector) encoding the second, non-specific dsRNA molecule is also administered to the cell to reduce or inhibit the adverse effects due to the possible induction of the interferon response upon administration of the dsRNA expression library to the cell, as is discussed above. See also, e.g., U.S. Ser. No. 10/425,006 filed 28 Apr. 2003, “Methods of Silencing Genes Without Inducing Toxicity”, C. Pachuk, incorporated herein by reference. The second dsRNA molecule differs from the dsRNA molecules encoded by the dsRNA expression library, in that it does not have substantial sequence identity to a target nucleic acid sequence (e.g., all or a region of a gene, a gene promoter, or a portion of a gene and its promoter), and is provided specifically to reduce or inhibit the interferon response or dsRNA-mediated toxicity; it is not provided to modulate the function of a cell, to modulate the expression of a target gene in a cell, or to modulate the biological activity of a target polypeptide in a cell. In some embodiments, the second, non-specific dsRNA molecule binds PKR and inhibits the dimerization and/or activation of PKR.

In some embodiments of these aspects, the dsRNA molecule of the invention having double-stranded regions with substantial sequence identity to a target nucleic acid sequence and the second, non-specific dsRNA molecule are a dsRNA molecules with one or more mismatched regions or one strand with two or more hairpin regions separated by single-stranded regions, as described herein.

In one embodiment of any of the above aspects of the invention, in transforming step (a), discussed above, the nucleic acid molecule (i.e., the dsRNA expression vector) is stably integrated into a chromosome of the cell. Integration of the dsRNA expression vector may be random or site-specific. Desirably integration is mediated by recombination or retroviral insertion. In addition, a single copy of the dsRNA expression vector is desirably integrated into the chromosome and is stably expressed. In another embodiment of any of the above aspects of the invention, in step (a) at least 50, more desirably 100; 500; 1000; 10,000; or 50,000 cells of the cell population are each transformed with a different nucleic acid molecule from the dsRNA expression library. Desirably, the expression library is derived from the transfected cells or cells of the same cell type as the transfected cells. In other embodiments, the population of cells is transformed with at least 5%, more desirably at least 25%, 50%, 75%, or 90%, and most desirably at least 95% of the dsRNA expression library.

In other embodiments of any of the above aspects of the invention, the dsRNA expression library contains cDNA molecules or randomized nucleic acid molecules. The dsRNA expression library may be a nuclear dsRNA expression library, in which case the dsRNA molecule encoded by the dsRNA expression vector is made in the nucleus. Alternatively, the dsRNA expression library may be a cytoplasmic dsRNA expression library, in which case the dsRNA molecule encoded by the dsRNA expression vector is made in the cytoplasm. In addition, the nucleic acid molecule from the dsRNA expression library may be made in vitro or in vivo. In addition, the identified nucleic acid sequence may be located in the cytoplasm or nucleus of the cell.

In still another embodiment of any of the above aspects of the invention, the nucleic acid sequence is contained in a vector, for example a dsRNA expression vector. The vector may then be transformed such that it is stably integrated into a chromosome of the cell, or it may function as an episomal (non-integrated) expression vector within the cell. In one embodiment, a vector that is integrated into a chromosome of the cell contains a promoter operably linked to a nucleic acid sequence encoding a hairpin or dsRNA molecule. In another embodiment, the vector does not contain a promoter operably linked to a nucleic acid sequence encoding a dsRNA molecule. In this latter embodiment, the vector integrates into a chromosome of a cell, such that an endogenous promoter is operably linked to a nucleic acid sequence from the vector that encodes the dsRNA molecule.

Desirably, the dsRNA expression vector comprises at least one RNA polymerase II promoter, for example, a human CMV-immediate early promoter (HCMV-IE) or a simian CMV (SCMV) promoter, and/or at least one RNA polymerase I promoter, and/or at least one RNA polymerase III promoter. Desirably, multiple promoters active in different subcellular compartments of a eukaryotic cell may be used; see further the teaching of “Multiple-Compartment Eukaryotic Expression Systems”, C. Pachuk and C. Satishchandran, U.S. Provisional Application Ser. No. 60/497,304, filed Aug. 22, 2003, incorporated herein by reference.

The promoter may also be a T7 promoter, in which case, the cell further comprises T7 polymerase. Alternatively, the promoter may be an SP6 promoter, in which case, the cell further comprises SP6 polymerase. The promoter may also be one convergent T7 promoter and one convergent SP6 promoter. A cell may be made to contain T7 or SP6 polymerase by transforming the cell with a T7 polymerase or an SP6 polymerase expression plasmid, respectively. In some embodiments, a T7 promoter or a RNA polymerase III promoter is operably linked to a nucleic acid sequence that encodes a short dsRNA (e.g., a dsRNA that is less than 200, 150, 100, 75, 50, or 25 nucleotides in length). In other embodiments, the promoter is a mitochondrial promoter that allows cytoplasmic transcription of the nucleic acid sequence in the vector (see, for example, the mitochondrial promoters described in WO 00/63364, filed Apr. 19, 2000). Alternatively, the promoter is an inducible promoter, such as a lac (Cronin et al. Genes & Development 15: 1506-1517, 2001), ara (Khlebnikov et al., J Bacteriol. 2000 December; 182(24):7029-34), ecdysone (Rheogene website), RU48 (mefepristone) (corticosteroid antagonist) (Wang X J, Liefer K M, Tsai. S, O'Malley B W, Roop D R, Proc Natl Acad Sci USA. 1999 Jul. 20; 96(15):8483-8), or tet promoter (Rendal et al., Hum. Gene Ther. 2002; 13(2):335-42 and Larnartina et al., Hum. Gene Ther. 2002; 13(2):199-210) or a promoter disclosed in WO 00/63364, filed Apr. 19, 2000. In desirable embodiments, the inducible promoter is not induced until all the episomal vectors are eliminated from the cell. The vector may also comprise a selectable marker.

In particular embodiments, the dsRNA molecule encoded by the dsRNA expression library is between 11 and 40 nucleotides in length and, in the absence of a second, non-specific dsRNA molecule, as is discussed above, may induce toxicity in vertebrate cells because its sequence has affinity for PKR or another protein in a dsRNA-mediated stress response pathway. In this instance, the second, non-specific dsRNA molecule can be administered to the cell to reduce or inhibit this toxicity.

In still other embodiments of any of the above aspects of the invention, the cell and the dsRNA expression vector each further comprise a loxP site and site-specific integration of the dsRNA expression vector into a chromosome of the cell occurs through recombination between the loxP sites. In addition, the method further involves rescuing the dsRNA expression vector through Cre-mediated double recombination, thereby facilitating integration of the dsRNA expression vector into the genome of the cell.

In yet another embodiment of any of the above aspects of the invention, the cell is derived from a parent cell, and is generated by (a) transforming a population of parent cells with a bicistronic plasmid expressing a selectable marker and a reporter gene, and comprising a loxP site; (b) selecting for a cell in which the plasmid is stably integrated; and (c) selecting for a cell in which one copy of the plasmid is stably integrated in a transcriptionally active locus. Desirably the selectable marker is G418 and the reporter gene is green fluorescent protein (GFP). These methods are disclosed in further detail in U.S. Published Application 2002/0132257 and European Published Application 1229134, “Use of post-transcriptional gene silencing for identifying nucleic acid sequences that modulate the function of a cell”, the teaching of which is hereby incorporated by reference.

Methods for Identifying Nucleic Acids of Interest by Transfecting Cells with dsRNA Molecules

In addition to the above screening methods that utilize a dsRNA expression library, the invention provides screening methods that utilize one or more dsRNA molecules having one or more double-stranded regions preferably two or more double-stranded regions), in which all or a portion of at least one double-stranded region has substantial sequence identity to a target nucleic acid sequence (e.g., all or a region of a gene, a gene promoter, or a portion of a gene and its promoter) and that reduce or inhibit expression of a target gene. If desired, one or more non-specific dsRNA molecules, as described above, can also be administered to inhibit the interferon response. Desirably, the method is carried out under conditions that inhibit or prevent an interferon response or dsRNA stress response.

In one such aspect, the invention features a method for identifying a nucleic acid sequence that modulates a detectable phenotype in a cell, (e.g., a gene that modulates the function of a cell, that modulates expression of a target gene in a cell, or that modulates the biological activity of a target polypeptide in a cell) in which the method involves (a) transforming a population of cells with a first dsRNA molecule or a dsRNA expression vector encoding the dsRNA molecule; and, when a dsRNA expression vector is used, (b) optionally selecting for a cell in which dsRNA molecule(s) is expressed; and (c) assaying for a modulation in the detectable phenotype of the cell. When expressed or present in the cell, the first dsRNA molecule, which has one or more double-stranded regions (preferably two or more double-stranded regions), and in which all or a portion of at least one double-stranded region has substantial sequence identity to at least one target nucleic acid sequence in the cell, and when cleaved by an endogenous or exogenously provided single-stranded ribonuclease that liberates the double-stranded region(s) of the dsRNA molecule, specifically reduces or inhibits the expression of a target gene in the cell, thereby resulting in a modulation in a detectable phenotype of the cell. In a desirable embodiment, the target nucleic acid sequence is assayed using DNA array technology. In a desirable embodiment, assaying for a modulation in the function of a cell involves measuring cell motility, apoptosis, cell growth, cell invasion, vascularization, cell cycle events, cell differentiation, cell dedifferentiation, neuronal cell regeneration, or the ability of a cell to support viral replication.

### Additional Embodiments of any of the Various Aspects of the Invention

In one embodiment of any of the above aspects of the invention, at least 2, more desirably 50; 100; 500; 1000; 10,000; or 50,000 cells of the population of cells are each transformed with a different dsRNA molecule or dsRNA expression vector encoding the dsRNA molecule. Desirably, at most one first dsRNA molecule having one or more double-stranded regions (preferably two or more double-stranded regions), in which all or a portion of at least one double-stranded region has substantial sequence identity to a target nucleic acid sequence (e.g., all or a region of a gene, a gene promoter, or a portion of a gene and its promoter), is inserted into each cell. In other embodiments, the population of cells is transformed with at least 5%, more desirably at least 25%, 50%, 75%, or 90%, and most desirably, at least 95% of the dsRNA expression library or dsRNA library. In still another embodiment, the method further involves identifying the nucleic acid sequence by amplifying and cloning the sequence. Desirably amplification of the sequence involves the use of the polymerase chain reaction (PCR).

### Desirable Vectors

In still another embodiment of any of the various aspects of the invention, the nucleic acid sequence is contained in a vector, for example, a dsRNA expression vector that encodes a dsRNA molecule of the invention. Desirably the dsRNA expression vector comprises at least one promoter. The promoter may be a T7 promoter, in which case, the cell further comprises T7 polymerase. Alternatively, the promoter may be an SP6 promoter, in which case, the cell further comprises SP6 polymerase. The promoter may also be one convergent T7 promoter and one convergent SP6 promoter. A cell may be made to contain T7 or SP6 polymerase by transforming the cell with a T7 polymerase or an SP6 polymerase expression plasmid, respectively. The vector may also comprise a selectable marker, for example hygromycin. In some embodiments, the same vector encodes the dsRNA molecule and the polymerase (e.g., a T7 or SP6 polymerase). Desirably, multiple promoters active in different subcellular compartments of a eukaryotic cell may be used; see further the teaching of “Multiple-Compartment Eukaryotic Expression Systems”, C. Pachuk and C. Satishchandran, U.S. Provisional Application Ser. No. 60/497,304, filed Aug. 22, 2003, incorporated herein by reference.

Desirably, in a vector for use in the methods of the invention, the sense strand and the antisense strand of the nucleic acid sequence are transcribed from the same nucleic acid sequence using two convergent promoters. In another desirable embodiment, in a vector for use in any of the above aspects of the invention, the nucleic acid sequence comprises an inverted repeat, such that upon transcription, the transcribed RNA forms a dsRNA molecule. In desirable embodiments, the dsRNA molecule has mismatched regions or one strand with two or more hairpin regions separated by single-stranded regions, as described herein.

Other desirable vectors have an origin of replication that enables the DNA vector to be replicated upon nuclear localization, such as the SV40 T origin, EBNA origin, or a mammalian origin. Desirably, the vector with the origin of replication or another vector or chromosome in the cell encodes an accessory factor such as SV40 TAg or EBNA that enables the vector to replicate in the cell.

Desirable dsRNA Molecules

Desirable methods of any of the above aspects use one or more dsRNA molecules (e.g., dsRNA molecule with mismatched regions or one strand with two or more hairpin regions separated by single-stranded regions, as described herein), or one or more vectors of the invention. In some embodiments, the dsRNA molecule contains coding sequence, non-coding sequence, or a combination thereof. For TGS applications, the dsRNA desirably includes a regulatory sequence (e.g., a transcription factor binding site, a promoter, and/or a 5′ or 3′ untranslated region (UTR) of an mRNA) and/or a coding sequence. For PTGS applications, the dsRNA desirably includes a regulatory sequence (e.g., a 5′ or 3′ untranslated region (UTR) of an mRNA) and/or a coding sequence. In some embodiments, the same dsRNA mediates both TGS and PTGS. In other embodiments, one or more dsRNA molecules that mediate TGS and one or more dsRNA molecules that mediate PTGS are used. In some embodiments, the dsRNA has 1, 2, 3, 4, 5, 6, or more constitutive transport element (CTE) sequences (e.g., a CTE from Mason-Pfizer Monkey virus). In certain embodiments, the dsRNA has one or more introns and/or a polyA tail. Desirably, the amount of dsRNA located in the cytoplasm of a cell is at least 24, 50, 75, 100, 200, 400, 600, or even 1000% greater for a dsRNA that has a CTE, intron, and/or polyA tail than for a control dsRNA lacking the CTE, intron, and/or polyA tail.

In other embodiments of any of the above aspects of the invention, the dsRNA molecules are derived from cDNA molecules or randomized nucleic acid sequences. In some embodiments, the dsRNA is located in the cytoplasm or nucleus. In some embodiments, some of the dsRNA transcripts are located in the cytoplasm, and some of the transcripts are located in the nucleus. Desirably, the dsRNA mediates both PTGS and TGS. In other embodiments, at least 50, 60, 70, 80, 90, 95, or 100% of the dsRNA molecules are located in the cytoplasm and thus can mediate PTGS. In still other embodiments, at least 50, 60, 70, 80, 90, 95, or 100% of the dsRNA molecules are located in the nucleus and can mediate TGS. In some embodiments, dsRNA molecules that mediate TGS comprise a region with substantial sequence identity to the promoter of a target gene. Other dsRNA molecules have, e.g., a region with substantial sequence identity to the promoter and a region substantially identical to the coding region of the target gene. The dsRNA molecule may be made in vitro or in vivo. In various embodiments, the identified nucleic acid sequence is located in the cytoplasm or nucleus of the cell.

In yet another embodiment, the dsRNA is at least 100, 500, 600, or 1000 nucleotides in length. In other embodiments, the dsRNA is at least 10, 20, 30, 40, 50, 60, 70, 80, or 90 nucleotides in length. In yet other embodiments, the number of nucleotides in the dsRNA is between 5-100 nucleotides, 15-100 nucleotides, 20-95 nucleotides, 25-90 nucleotides, 35-85 nucleotides, 45-80 nucleotides, 50-75 nucleotides, or 55-70 nucleotides, inclusive. In still other embodiments, the number of nucleotides in the dsRNA is contained in one of the following ranges: 5-15 nucleotides, 15-20 nucleotides, 19-26 nucleotides, 20-25 nucleotides, 25-35 nucleotides, 35-45 nucleotides, 45-60 nucleotides, 60-70 nucleotides, 70-80 nucleotides, 80-90 nucleotides, or 90-100 nucleotides, inclusive. In other embodiments, the dsRNA contains less than 50,000; 10,000; 5,000; or 2,000 nucleotides. In addition, the dsRNA may contain a sequence that is less than a full length RNA sequence. In other desirable embodiments, the double-stranded region in the dsRNA (e.g., a long dsRNA) contains between 11 and 30 nucleotides, inclusive; between 19 and 26 nucleotides, inclusive; over 30 nucleotides; or over 200 nucleotides. In desirable embodiments, the double-stranded region in the short dsRNA contains between 11 and 30 nucleotides, inclusive; or between 19 and 26 nucleotides, inclusive.

In some embodiments, the dsRNA molecule (e.g., the first dsRNA molecule) is 20 to 30 nucleotides (e.g., 20, 21, 22, 23, 24, 25, 26, 27, or 28 nucleotides) in length. In particular embodiments, the first dsRNA molecule is between 11 and 40 nucleotides in length and, in the absence of the non-specific dsRNA molecule described above, may induce toxicity in vertebrate cells because its sequence has affinity for PKR or another protein in a dsRNA mediated stress response pathway. The non-specific dsRNA molecule of the invention inhibits this toxicity.

In other embodiments, the dsRNA molecule is derived from a cell or a population of cells and is used to transform another cell population of either the same cell type or a different cell type. In desirable embodiments, the transformed cell population contains cells of a cell type that are related to the cell type of the cells from which the dsRNA was derived (e.g., the transformation of cells of one neuronal cell type with the dsRNA derived from cells of another neuronal cell type). In yet other embodiments of any of these aspects, the dsRNA molecule contains one or more contiguous or non-contiguous positions that are randomized (e.g., by chemical or enzymatic synthesis using a mixture of nucleotides that may be added at the randomized position). In still other embodiments, the dsRNA molecule contains a randomized nucleic acid sequence in which segments of ribonucleotides and/or deoxyribonucleotides are ligated to form the dsRNA molecule. In desirable embodiments, the agent, nucleic acid molecule, dsRNA molecule, or dsRNA expression vector is a nucleic acid molecule of the invention (e.g., a partial or full hairpin, or a vector encoding a partial or full hairpin).

In other embodiments of any of the various aspects of the invention, the dsRNA molecule of the invention specifically hybridizes to a target nucleic acid sequence (e.g., all or a region of a gene, a gene promoter, or a gene and gene promoter sequence) in a cell or biological sample, but does not substantially hybridize to non-target molecules, which include other nucleic acid sequences in the cell or biological sample having a sequence that is less than 99, 95, 90, 80, or 70% identical or complementary to that of the target nucleic acid sequence. Desirably, the amount of the non-target molecules hybridized to, or associated with, the dsRNA molecule, as measured using standard assays, is 2-fold, desirably 5-fold, more desirably 10-fold, and most desirably 50-fold lower than the amount of the target nucleic acid sequence hybridized to, or associated with, the dsRNA molecule. In other embodiments, the amount of a target nucleic acid sequence hybridized to, or associated with, the dsRNA molecule, as measured using standard assays, is 2-fold, desirably 5-fold, more desirably 10-fold, and most desirably 50-fold greater than the amount of a control nucleic acid sequence hybridized to, or associated with, the dsRNA molecule. Desirably, the dsRNA molecule of the invention only hybridizes to one target nucleic acid sequence from a cell or biological sample under denaturing, high stringency hybridization conditions, as defined herein. In certain embodiments, the dsRNA molecule has one or more double-stranded regions (preferably two or more double-stranded regions), in which all or a portion of at least one double-stranded region has substantial sequence identity (e.g., at least 80, 90, 95, 98, or 100% sequence identity) to only one target nucleic acid sequence from a cell or biological sample.

In other embodiments, the dsRNA molecule has one or more double-stranded regions (preferably two or more double-stranded regions), in which all or a portion of at least one double-stranded region has substantial sequence identity to multiple RNA molecules, such as RNA molecules from the same gene family. In yet other embodiments, the dsRNA molecule has one or more double-stranded regions (preferably two or more double-stranded regions), in which all or a portion of at least one double-stranded region has substantial sequence identity to distinctly different mRNA sequences from genes that are similarly regulated (e.g., developmental, chromatin remodeling, or stress response induced). In other embodiments, the dsRNA molecule is homologous to a large number of RNA molecules, such as a dsRNA designed to induce a stress response or apoptosis (e.g., a dsRNA designed to kill cancer cells or other unhealthy or excess cells).

In other embodiments, the percent decrease in the expression of a target gene is at least 2, 5, 10, 20, or 50 fold greater than the percent decrease in the expression of a non-target or control gene. Desirably, the dsRNA molecule reduces or inhibits the expression of a target gene but has negligible, if any, effect on the expression of other genes in the cell. A desired characteristic of the dsRNA molecule is that the double-stranded region(s), when liberated from the dsRNA molecule by an endogenous or exogenously provided ribonuclease, has little, if any, affinity for a nucleic acid molecule with a random nucleic acid sequence (i.e., a nucleic acid sequence that is not related to or associated with a target gene). Desirably, the dsRNA molecules of the invention are substantially non-homologous to a naturally-occurring essential mammalian gene or to all the essential mammalian genes (see, for example, WO 00/63364). In some embodiments, the dsRNA molecule does not adversely affect the function of an essential gene. In other embodiments, the dsRNA molecule adversely affects the function of an essential gene, e.g., a gene in a cancer cell.

Desirably, the non-specific dsRNA molecule described above inhibits the dimerization of PKR or another protein in a dsRNA-mediated stress response pathway in a cell or animal by at least 10, 20, 30, 40, 50, 60, 70, 80, 90, or 95% compared to the amount of dimerization of the protein in a control cell or animal not administered the non-specific dsRNA molecule, as measured using standard methods such as those described herein. In some embodiments, the non-specific dsRNA molecule includes a region of randomized sequence, or the entire non-specific dsRNA molecule contains randomized sequence. In various embodiments, the non-specific dsRNA does not substantially decrease the expression of a target gene in a cell or biological sample (e.g., the non-specific dsRNA decreases expression of a target gene by less than 60, 40, 30, 20, or 10%). In certain embodiments, the sequence of the non-specific dsRNA is less than 80, 70, 60, 50, 30, 20, or 10% identical to or complementary to that of a nucleic acid sequence (e.g., a gene or gene promoter) in a cell or biological sample. In particular embodiments, multiple non-specific dsRNA molecules or multiple vectors encoding non-specific dsRNA molecules are administered to a cell, and less than 70, 60, 50, 30, 20, or 10% of the non-specific dsRNA molecules have a sequence that is at least 50, 70, 80, or 90% identical to or complementary to that of a nucleic acid sequence (e.g., a target gene or its promoter) in the cell.

In other embodiments of any of various aspects of the invention, at most one molecular species of the dsRNA molecule of the invention is inserted into each cell. In other embodiments, at most one vector encoding a dsRNA molecule of the invention is stably integrated into the genome of each cell and one dsRNA molecule of the invention is stably expressed therefrom. In various embodiments, the dsRNA molecule of the invention is active in the nucleus of the transformed cell and/or is active in the cytoplasm of the transformed cell. In various embodiments, at least 1, 10, 20, 50, 100, 500, or 1000 cells or all of the cells in the population are selected as cells that contain or express a dsRNA (e.g., a long dsRNA). In some embodiments, at least 1, 10, 20, 50, 100, 500, or 1000 cells or all of the cells in the population are assayed for a modulation of a detectable phenotype, e.g., modulation in the function of the cell, a modulation in the expression of a target nucleic acid (e.g., an endogenous or pathogen gene) in the cell, and/or a modulation in the biological activity of a target protein (e.g., an endogenous or pathogen protein) in the cell.

Desirable RNA Polymerases

In certain embodiments, an RNA dependent-RNA polymerase is expressed in a cell or animal into which a dsRNA or a vector encoding a dsRNA is introduced. The RNA dependent-RNA polymerase amplifies the dsRNA and desirably increases the number of dsRNA molecules in the cell or animal by at least 25, 50, 100, 200, 500, 1000, 5000, or even 10000%. In various embodiments, the RNA dependent-RNA polymerase is naturally expressed by the cell or animal or is encoded by the same or a different vector that encodes the dsRNA. Exemplary RNA dependent-RNA polymerases include viral, plant, invertebrate, or vertebrate (e.g., mammalian or human) RNA dependent-RNA polymerases. Providing an RNA dependent-RNA polymerase (RdRp) is especially important in those embodiments of the invention that utilize partial hairpin dsRNAs which are extended in vitro or in vivo with an RNA dependent-RNA polymerase, unless the cells or system in which the partial hairpin is utilized contains an endogenous RdRp. See Table 1, which provides a non-exclusive list of RNA dependent-RNA polymerases useful in the methods of the invention.

TABLE 1
RNA dependent RNA polymerases

| | Genbank |
|---|---|
| Source of Polymerase | Accession No. |
| Turnip Crinkle Virus | NC_003821 |
| Taura Syndrome Virus | NC_003005 |
| L. esculentum mRNA for RNA-directed RNA | Y10403 |
| polymerase | |
| Perina Nuda Picorna-like Virus | NC_003113 |
| Dengue Virus Type 2 Strain TSV01 | AY037116 |
| Caenorhabditis elegans RNA-directed RNA | AF159143 |
| polymerase related EGO-1 (ego-1) mRNA | |
| Caenorhabditis elegans RRF-1 (rrf-1) mRNA | AF159144 |
| Hepatitis C virus | NC_001433 |
| Cucumber Leaf Spot Virus putative RNA- | AY038365 |
| dependent RNA polymerase gene | |
| Pseudomonas phage phi-6 segment M | NC_003716 |
| Pseudomonas phage phi-6 segment L | NC_003715 |
| Pseudomonas phage phi-6 segment S | NC_003714 |
| Chain P, RNA Dependent RNA Polymerase from | 1HI0P |
| dsRNA Bacteriophage Phi6 Plus Initiation | |
| Complex | |
| Bovine Viral Diarrhea Virus Genotype 2 | NC_002032 |
| Putative Polyprotein [Bovine Viral Diarrhea Virus | NP_044731 |
| Genotype 2] | |

Optional Administration of Target Gene

In some embodiments, a target gene (e.g., a pathogen or endogenous target gene) or a region from a target gene (e.g., a region from an intron, exon, untranslated region, promoter, or coding region) is introduced into the cell or animal. For example, this target gene can be inserted into a vector (e.g., a vector that desirably can integrate into the genome of a cell) and then administered to the cell or animal. Desirably, the administration of one or more copies of the target gene enhances the amplification of a dsRNA molecule (e.g., a dsRNA molecule having one or more double-stranded regions, preferably two or more double-stranded regions, in which all or a portion of at least one double-stranded region has substantial sequence identity to the target gene) administered to the cell or animal or enhances the amplification of cleavage products from this dsRNA molecule.

### Optional Methods to Inhibit an Interferon Response

In some embodiments, a component of the interferon response or dsRNA stress response pathway (e.g., PKR, human beta interferon, and/or 2′5′OAS) is inhibited in the cell or animal. In various embodiments, one or more components are inhibited using dsRNA-mediated gene silencing, antisense-mediated gene silencing, ribozyme-mediated gene silencing, or genetic knockout methods. Additionally, one or more IRE sequences and/or one or more transcription factors that bind IRE sequences, such as STAT1, can be optionally silenced or mutated. In various embodiments, one or more nucleic acid sequences that encode proteins that block the PKR response, such as the Vaccinia virus protein E3, the cellular protein P58^{IPK}, or a Hepatitis C E2 protein, are administered to the cell or animal.

### Desirable Methods of Administration of Nucleic Acid Sequences

In some embodiments, the dsRNA or dsRNA expression vector is complexed with one or more cationic lipids or cationic amphiphiles, such as the compositions disclosed in U.S. Pat. No. 4,897,355 (Eppstein et al., filed Oct. 29, 1987), U.S. Pat. No. 5,264,618 (Felgner et al., filed Apr. 16, 1991) or U.S. Pat. No. 5,459,127 (Felgner et al., filed Sep. 16, 1993). In other embodiments, the dsRNA or dsRNA expression vector is complexed with a liposome/liposomic composition that includes a cationic lipid and optionally includes another component, such as a neutral lipid (see, for example, U.S. Pat. No. 5,279,833 (Rose), U.S. Pat. No. 5,283,185 (Epand), and U.S. Pat. No. 5,932,241 (Gorman)). In other embodiments, the dsRNAs or dsRNA expression constructs are complexed with the multifunctional molecular complexes of U.S. Pat. No. 5,837,533, U.S. Pat. No. 6,127,170, and U.S. Pat. No. 6,379,965 (Boutin), or the multifunctional molecular complexes or oil/water cationic amphiphile emulsions of PCT/US03/14288, filed May 6, 2003 (Satishchandran).

In yet other embodiments, the dsRNA or dsRNA expression vector is complexed with any other composition that is devised by one of ordinary skill in the fields of pharmaceutics and molecular biology. In some embodiments, the dsRNA or the vector is not complexed with a cationic lipid.

Transformation/transfection of the cell may occur through a variety of means including, but not limited to, lipofection, DEAE-dextran-mediated transfection, microinjection, protoplast fusion, calcium phosphate precipitation, viral or retroviral delivery, electroporation, or biolistic transformation. The RNA or RNA expression vector (DNA) may be naked RNA or DNA or local anesthetic complexed RNA or DNA (Pachuk et al., supra). In yet another embodiment, the cell is not a C. elegans cell. Desirably the vertebrate (e.g., mammalian) cell has been cultured for only a small number of passages (e.g., less than 30 passages of a cell line that has been directly obtained from American Type Culture Collection), or are primary cells. In addition, desirably the vertebrate (e.g., mammalian) cell is transformed with dsRNA that is not complexed with cationic lipids.

Desirable Cells

In still further embodiments of any aspect of the invention, the cell is a plant cell or an animal cell. Desirably the animal cell is an invertebrate or vertebrate cell (e.g., a mammalian cell, for example, a human cell). The cell may be ex vivo or in vivo. The cell may be a gamete or a somatic cell, for example, a cancer cell, a stem cell, a cell of the immune system, a neuronal cell, a muscle cell, or an adipocyte. In some embodiments, one or more proteins involved in gene silencing, such as Dicer or Argonaut, are overexpressed or activated in the cell or animal to increase the amount of inhibition of gene expression.

Some Advantages of the Present Invention

The present methods provide numerous advantages for the silencing of genes in cells and animals. For example, in other dsRNA delivery systems some dsRNA molecules induce an interferon response (Jaramillo et al., Cancer Invest. 13:327-338, 1995). Induction of an interferon response is not desired because it can lead to cell death and possibly prevent gene silencing. Thus, a significant advantage of the present invention is that the dsRNA delivery methods described herein are performed such that an interferon response is inhibited or prevented. These methods allow dsRNA to be used in clinical applications for the prevention or treatment of disease or infection without the generation of adverse side-effects due to dsRNA-induced toxicity. The use of both short and long dsRNA molecules in some embodiments of the present methods may also have improved efficiency for silencing genes, as compared to previous methods that use only short dsRNA molecules.

### DEFINITIONS

By “agent that provides an at least partially double-stranded RNA” is meant a composition that generates an at least partially double-stranded (ds)RNA in a cell or animal. For example, the agent can be a dsRNA, a single-stranded RNA molecule that assumes a double-stranded conformation inside the cell or animal (e.g., a hairpin), or a combination of two single-stranded RNA molecules that are administered simultaneously or sequentially and that assume a double-stranded conformation inside the cell or animal. Other exemplary agents include a DNA molecule, plasmid, viral vector, or recombinant virus encoding an at least partially dsRNA. Other agents are disclosed in WO 00/63364, filed Apr. 19, 2000. In some embodiments, the agent includes between 1 ng and 20 mg, 1 ng to 1 ug, 1 ug to 1 mg, or 1 mg to 20 mg of DNA and/or RNA.

By “alteration in the level of gene expression” is meant a change in transcription, translation, or mRNA or protein stability, such that the overall amount of a product of the gene, i.e., mRNA or polypeptide, is increased or decreased.

By “apoptosis” is meant a cell death pathway wherein a dying cell displays a set of well-characterized biochemical hallmarks that include cytolemmal membrane blebbing, cell soma shrinkage, chromatin condensation, nuclear disintegration, and DNA laddering. There are many well-known assays for determining the apoptotic state of a cell, including, and not limited to: reduction of MTT tetrazolium dye, TUNEL staining, Annexin V staining, propidium iodide staining, DNA laddering, PARP cleavage, caspase activation, and assessment of cellular and nuclear morphology. Any of these or other known assays may be used in the methods of the invention to determine whether a cell is undergoing apoptosis.

By “assaying” is meant analyzing the effect of a treatment, be it chemical or physical, administered to whole animals, cells, tissues, or molecules derived therefrom. The material being analyzed may be an animal, a cell, a tissue, a lysate or extract derived from a cell, or a molecule derived from a cell. The analysis may be, for example, for the purpose of detecting altered cell function, altered gene expression, altered endogenous RNA stability, altered polypeptide stability, altered polypeptide levels, or altered polypeptide biological activity. The means for analyzing may include, for example, antibody labeling, immunoprecipitation, phosphorylation assays, glycosylation assays, and methods known to those skilled in the art for detecting nucleic acid molecules. In some embodiments, assaying is conducted under selective conditions.

By “bacterial infection” is meant the invasion of a host animal by pathogenic bacteria. For example, the infection may include the excessive growth of bacteria that are normally present in or on the body of a animal or growth of bacteria that are not normally present in or on the animal. More generally, a bacterial infection can be any situation in which the presence of a bacterial population(s) is damaging to a host animal. Thus, a animal is “suffering” from a bacterial infection when an excessive amount of a bacterial population is present in or on the animal's body, or when the presence of a bacterial population(s) is damaging the cells or other tissue of the animal. In one embodiment, the number of a particular genus or species of bacteria is at least 2, 4, 6, or 8 times the number normally found in the animal. The bacterial infection may be due to gram positive and/or gram negative bacteria.

By “Bernie Moss hairpin” or “BM hairpin” is meant a hairpin structure as described in, e.g., Fuerst and Moss, “Structure and stability of mRNA synthesized by vaccinia virus-encoded bacteriophage T7 RNA Polymerase in mammalian cells”, J. Mol. Biol. 206:333-348, 1989. The presence of a BM hairpin at the 5′ terminus of an RNA transcript stabilizes the proximate transcript region and protects the 5′ terminus of the transcript from degradation and/or loss due to staggered initiation of transcription.

By “cistron” or “transcription unit” is meant a unit in which transcription occurs. Usually a “cistron” or “transcription unit” means a promoter sequence operably linked to a nucleic acid sequence to be transcribed, optionally with a terminator or polyadenylation signal.

By “Cre-mediated double recombination” is meant two nucleic acid recombination events involving loxP sites that are mediated by Cre recombinase. A Cre-mediated double recombination event can occur, for example, as disclosed in more detail in U.S. Published Application 2002/0132257, and, e.g., in FIG. 1 thereof.

By “a decrease” is meant a lowering in the level of: a) protein (e.g., as measured by ELISA or Western blot analysis); b) reporter gene activity (e.g., as measured by reporter gene assay, for example, β-galactosidase, green fluorescent protein, or luciferase activity); c) mRNA (e.g., as measured by RT-PCR or Northern blot analysis relative to an internal control, such as a “housekeeping” gene product, for example, β-actin or glyceraldehyde 3-phosphate dehydrogenase (GAPDH)); or d) cell function, for example, as assayed by the number of apoptotic, mobile, growing, cell cycle arrested, invasive, differentiated, or dedifferentiated cells in a test sample. In all cases, the lowering is desirably by at least 20%, more desirably by at least 30%, 40%, 50%, 60%, 75%, and most desirably by at least 90%. As used herein, a decrease may be the direct or indirect result of PTGS, TGS, or another gene silencing event.

By “dsRNA” is meant a nucleic acid molecule containing a region of 17, 18 or more, preferably at least 19 or more basepairs that are in a double-stranded conformation. In various embodiments, the dsRNA consists entirely of ribonucleotides or consists of a mixture of ribonucleotides and deoxynucleotides, such as the RNA/DNA hybrids disclosed, for example, by WO 00/63364, filed Apr. 19, 2000, or U.S. Ser. No. 60/130,377, filed Apr. 21, 1999. The dsRNA may be a single molecule with regions of self-complementarity such that nucleotides in one segment of the molecule base pair with nucleotides in another segment of the molecule. In various embodiments, a dsRNA that consists of a single molecule consists entirely of ribonucleotides or includes a region of ribonucleotides that is complimentary to a region of deoxyribonucleotides. Alternatively, the dsRNA may be a duplex dsRNA, i.e., including two different strands that have a region of complementarity to each other. In various embodiments, both strands consist entirely of ribonucleotides, one strand consists entirely of ribonucleotides and one strand consists entirely of deoxyribonucleotides, or one or both strands contain a mixture of ribonucleotides and deoxyribonucleotides. Desirably, the regions of complementarity are at least 70, 80, 90, 95, 98, or 100% complimentary. Desirably, the region of the dsRNA that is present in a double-stranded conformation includes at least 19, 20, 30, 50, 75, 100, 200, 500, 1000, 2000, or 5000 nucleotides, or includes all of the nucleotides in a cDNA being represented in the dsRNA. In some embodiments, the dsRNA does not contain any single-stranded regions, such as single-stranded ends, or the dsRNA is a hairpin. In other embodiments, the dsRNA has one or more single-stranded regions or overhangs. In some embodiments, the dsRNA will be duplex RNA having double-stranded regions separated by mismatched regions that exist in single-stranded conformation. In some embodiments, the dsRNA will be a single RNA strand which assumes a hairpin or stem-loop structure having double-stranded regions separated by mismatched, single-stranded regions. In some embodiments, the dsRNA will comprise a series of hairpin or stem-loop structures separated by single-stranded “spacer” regions. Desirably, at least a portion of one or more double-stranded regions, or one or more entire double-stranded regions in any of the above embodiments will have sequence identity to a sequence of at least about 17, 18, or 19 to about 30 contiguous nucleotides of a target nucleotide, desirably about 19 to about 27, about 20 to about 27, about 21 to about 26, or about 21 to about 23 nucleotides of a target sequence. Desirably, there will be single-stranded regions located 5′, 3′, or both 5′ and 3′ to such double-stranded region(s) that can be cleaved to yield siRNAs (short interfering dsRNAs) of the desired length independent of Dicer or other similar enzymes which cleave dsRNA. Desirable RNA/DNA hybrids include a DNA strand or region that is an antisense strand or region (e.g., has at least 70, 80, 90, 95, 98, or 100% complementarity to a target nucleic acid) and an RNA strand or region that is a sense strand or region (e.g., has at least 70, 80, 90, 95, 98, or 100% identity to a target nucleic acid), or vice versa. In various embodiments, the RNA/DNA hybrid is made in vitro using enzymatic or chemical synthetic methods such as those described herein, or those described in WO 00/63364, filed Apr. 19, 2000 or U.S. Ser. No. 60/130,377, filed Apr. 21, 1999. In other embodiments, a DNA strand synthesized in vitro is complexed with an RNA strand made in vivo or in vitro before, after, or concurrent with the transformation of the DNA strand into the cell. In yet other embodiments, the dsRNA is a single circular nucleic acid containing a sense and an antisense region, or the dsRNA includes a circular nucleic acid and either a second circular nucleic acid or a linear nucleic acid (see, for example, WO 00/63364, filed Apr. 19, 2000 or U.S. Ser. No. 60/130,377, filed Apr. 21, 1999). Exemplary circular nucleic acids include lariat structures in which the free 5′ phosphoryl group of a nucleotide becomes linked to the 2′ hydroxyl group of another nucleotide in a loop back fashion. Desirable dsRNAs include the “forced hairpins” and “partial hairpins” as taught in U.S. Provisional Application 60/399,998, “Use of Double-Stranded RNA for Identifying Nucleic Acid Sequences that Modulate the Function of a Cell”, filed Jul. 31, 2003, and PCT/US03 . . . . “Double-stranded RNA Structures and Constructs and Methods for Generating and Using the Same”, filed Jul. 31, 2003, incorporated herein by reference.

In other embodiments, the dsRNA includes one or more modified nucleotides in which the 2′ position in the sugar contains a halogen (such as flourine group) or contains an alkoxy group (such as a methoxy group) which increases the half-life of the dsRNA in vitro or in vivo compared to the corresponding dsRNA in which the corresponding 2′ position contains a hydrogen or an hydroxyl group. In yet other embodiments, the dsRNA includes one or more linkages between adjacent nucleotides other than a naturally-occurring phosphodiester linkage. Examples of such linkages include phosphoramide, phosphorothioate, and phosphorodithioate linkages. In other embodiments, the dsRNA contains one or two capped strands or no capped strands, as disclosed, for example, by WO 00/63364, filed Apr. 19, 2000 or U.S. Ser. No. 60/130,377, filed Apr. 21, 1999. In other embodiments, the dsRNA contains coding sequence or non-coding sequence, for example, a regulatory sequence (e.g., a transcription factor binding site, a promoter, or a 5′ or 3′ untranslated region (UTR) of an mRNA). Additionally, the dsRNA can be any of the at least partially double-stranded RNA molecules disclosed in WO 00/63364, filed Apr. 19, 2000 (see, for example, pages 8-22). Any of the dsRNA molecules may be expressed in vitro or in vivo using the methods described herein, or using standard methods, such as those described in WO 00/63364, filed Apr. 19, 2000 (see, for example, pages 16-22).

By “dsRNA expression library” is meant a collection of nucleic acid expression vectors containing nucleic acid sequences, for example, cDNA sequences or randomized nucleic acid sequences that are capable of forming a dsRNA (dsRNA) upon expression of the nucleic acid sequence. Desirably the dsRNA expression library contains at least 10,000 unique nucleic acid sequences, more desirably at least 50,000; 100,000; or 500,000 unique nucleic acid sequences, and most desirably, at least 1,000,000 unique nucleic acid sequences. By a “unique nucleic acid sequence” is meant that a nucleic acid sequence of a dsRNA expression library has desirably less than 50%, more desirably less than 25% or 20%, and most desirably less than 10% nucleic acid identity to another nucleic acid sequence of a dsRNA expression library when the full length sequence is compared. Sequence identity is typically measured using BLAST® (Basic Local Alignment Search Tool) or BLAST®2 with the default parameters specified therein (see, Altschul et al., J. Mol. Biol. 215:403-410 (1990); and Tatiana et al., FEMS Microbiol. Lett. 174:247-250 (1999)). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

The preparation of cDNAs for the generation of dsRNA expression libraries is described, e.g., in U.S. Published Application 2002/0132257 and European Published Application 1229134, “Use of post-transcriptional gene silencing for identifying nucleic acid sequences that modulate the function of a cell”, the teaching of which is hereby incorporated by reference. A randomized nucleic acid library may also be generated as described, e.g., in U.S. Pat. No. 5,639,595, the teaching of which is hereby incorporated by reference, and utilized for dsRNA-mediated functional genomics applications. The dsRNA expression library may contain nucleic acid sequences that are transcribed in the nucleus or that are transcribed in the cytoplasm of the cell. A dsRNA expression library may be generated using techniques described herein.

By an “expression construct”, “expression vector”, “dsRNA expression construct”, or “dsRNA expression vector” is meant any double-stranded DNA or double-stranded RNA designed to transcribe an RNA, e.g., a construct that contains at least one promoter operably linked to a downstream gene or coding region of interest (e.g., a cDNA or genomic DNA fragment that encodes a protein, optionally, operatively linked to sequence lying outside a coding region, an antisense RNA coding region, or RNA sequences lying outside a coding region, or any RNA of interest). Transfection or transformation of the expression construct into a recipient cell allows the cell to express RNA or protein encoded by the expression construct. An expression construct may be a genetically engineered plasmid, virus, or an artificial chromosome derived from, for example, a bacteriophage, adenovirus, retrovirus, poxvirus, or herpesvirus. An expression construct can be replicated in a living cell, or it can be made synthetically.

By “full RNA hairpin” is meant a hairpin without a single-stranded overhang.

By “function of a cell” is meant any cell activity that can be measured or assessed. Examples of cell function include, but are not limited to, cell motility, apoptosis, cell growth, cell invasion, vascularization, cell cycle events, cell differentiation, cell dedifferentiation, neuronal cell regeneration, and the ability of a cell to support viral replication. The function of a cell may also be to affect the function, gene expression, or the polypeptide biological activity of another cell, for example, a neighboring cell, a cell that is contacted with the cell, or a cell that is contacted with media or other extracellular fluid in which the cell is contained.

By “gene of a pathogen” is meant a gene associated with a biological activity of a pathogenic cell or virus, e.g., a bacterium, a protozoan, or a parasite. Exemplary genes associated with a biological activity of a pathogen are genes for replication and/or pathogenesis of said pathogen, or a gene encoding a cellular receptor necessary for a host cell, e.g., a mammalian cell, to be infected with the pathogen.

By “high stringency conditions” is meant hybridization in 2×SSC at 40° C. with a DNA probe length of at least 40 nucleotides. For other definitions of high stringency conditions, see F. Ausubel et al., Current Protocols in Molecular Biology, pp. 6.3.1-6.3.6, John Wiley & Sons, New York, N.Y., 1994, hereby incorporated by reference.

By “isolated nucleic acid,” “nucleic acid sequence,” “nucleic acid molecule,” “dsRNA nucleic acid sequence,” or “dsRNA nucleic acid” is meant a nucleic acid molecule, or a portion thereof, that is free of the genes that, in the naturally-occurring genome of the organism from which the nucleic acid sequence of the invention is derived, flank the gene, or free of the flanking sequences and other cellular components that would accompany an RNA molecule in the naturally-occurring cell or organism. The term therefore includes, for example, a recombinant DNA, with or without 5′ or 3′ flanking sequences that is incorporated into a vector, for example, dsRNA expression vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences.

By “an increase” is meant a rise in the level of: (a) protein (e.g., as measured by ELISA or Western blot analysis); (b) reporter gene activity (e.g., as measured by reporter gene assay, for example, 3-galactosidase, green fluorescent protein, or luciferase activity); (c) mRNA (e.g., as measured by RT-PCR or Northern blot analysis relative to an internal control, such as a “housekeeping” gene product, for example, β-actin or glyceraldehyde 3-phosphate dehydrogenase (GAPDH)); or (d) cell function, for example, as assayed by the number of apoptotic, mobile, growing, cell cycle arrested, invasive, differentiated, or dedifferentiated cells in a test sample.

Desirably, the increase is by at least 1.5-fold to 2-fold, more desirably by at least 3-fold, and most desirably by at least 5-fold. As used herein, an increase may be the indirect result of PTGS, TGS, or another gene silencing event. For example, the dsRNA may inhibit the expression of a protein, such as a suppressor protein, that would otherwise inhibit the expression of another nucleic acid molecule.

By “long dsRNA” or “dsRNA of the invention” is meant a dsRNA that is at least 20, 30, 40, 50, 100, 200, 500, 1000, 2000, 50000, 10000, or more nucleotides in length. In some embodiments, the long dsRNA has a double-stranded region of between 100 to 10000, 100 to 1000, 200 to 1000, or 200 to 500 contiguous nucleotides, inclusive. In desirable embodiments, the double-stranded region is between 11 to 45, 11 to 40, 11 to 30, 11 to 20, 15 to 20, 15 to 18, 20 to 25, 21 to 23, 25 to 30, or 30 to 40 contiguous nucleotides in length, inclusive. In some embodiments, the long dsRNA is a single strand which achieves a double-stranded structure by virtue of regions of self-complementarity (e.g., inverted repeats or tandem sense and antisense sequences) that result in the formation of a hairpin structure. In one embodiment, the long dsRNA molecule does not produce a functional protein or is not translated. For example, the long dsRNA may be designed not to interact with cellular factors involved in translation. Exemplary long dsRNA molecules lack a poly-adenylation sequence, a Kozak region necessary for protein translation, an initiating methionine codon, and/or a cap structure. In other embodiments, the dsRNA molecule has a cap structure, one or more introns, and/or a polyadenylation sequence. Other such long dsRNA molecules include RNA/DNA hybrids. Other dsRNA molecules that may be used in the methods of the invention and various means for their preparation and delivery are described in WO 00/63364, filed Apr. 19, 2000, the teaching of which is incorporated herein by reference.

By “mismatched region” is meant a region that includes at least one nucleotide of a dsRNA that is not involved in base-pairing and wherein the unpaired nucleotide(s) is flanked by double-stranded regions (i.e., the nucleotide does not base-pair with other nucleotides in the mismatched region and does not base-pair with other nucleotides in other regions of the dsRNA). For example, the nucleotides of the mismatched region are unable to form a base-pair due to an insertion of a nucleotide, a deletion of a nucleotide, or due to steric constraints. Typically, a single mismatch, i.e., a one nucleotide insertion or deletion in one strand will result in a region of four nucleotides which will not participate in basepairing. In desirable embodiments, the mismatched region includes at least one nucleotide in one strand of a duplex dsRNA that is not involved in base-pairing (i.e., the nucleotide does not base-pair with other nucleotides in the same strand and does not base-pair with other nucleotides in the other strand). In some embodiments, the mismatched region includes at least two nucleotides (e.g., at least one nucleotide from each strand) of a duplex dsRNA that are not involved in base-pairing. In some embodiments, the mismatched region includes at least one nucleotide in a hairpin dsRNA that is not involved in base-pairing (i.e., the nucleotide does not base-pair with either other nucleotides in the mismatched region and does not base-pair with other nucleotides in other regions of the dsRNA). In desirable embodiments, there will be between about 4 and 10 nucleotides, about 4 to 20, about 4 to 50, or about 4 to about 100 nts in a mismatched region. In some embodiments, a mismatched region may include more than 100 nts, e.g., several hundred to a thousand nts. A mismatched region as defined herein includes not only regions of true nucleotide mismatch, e.g., a sequence of AAAAA residues vis-a-via a sequence of CCCCC residues, but also regions which are single-stranded because of steric constraints such as nucleotides in the region of a single nucleotide insertion or deletion in only one of two strands, or nucleotides in single-stranded “shoulder” regions flanking the stem region of a stem-loop structure, such as in FIG. 5F. Mismatched regions, particularly longer mismatched regions, may themselves include stem-loop or other structures. Desirably, in various embodiments, at least 10, 20, 40, 50, 60, 70, 80, 90, 95, 99, or 100% of the nucleotides in the mismatched region do not participate in base-pairing. Desirably, one or more mismatched regions of a dsRNA (e.g., 2, 3, 4, 5, 7, 8, 9, 10, 11, 12, 15, 18, 20, or more mismatched regions) are cleaved by an enzyme (e.g., an endogenous or exogenous RNase expressed in a cell, tissue, organ, or mammal in which gene silencing is desired).

By “modulates” is meant changing, either by a decrease or an increase. As used herein, desirably a nucleic acid molecule decreases the function of a cell, the expression of a target nucleic acid molecule in a cell, or the biological activity of a target polypeptide in a cell by least 20%, more desirably by at least 30%, 40%, 50%, 60% or 75%, and most desirably by at least 90%. Also as used herein, desirably a nucleic acid molecule increases the function of a cell, the expression of a target nucleic acid molecule in a cell, or the biological activity of a target polypeptide in a cell by at least 1.5-fold to 2-fold, more desirably by at least 3-fold, and most desirably by at least 5-fold.

By “multiple cloning site” is meant a known sequence within a DNA plasmid construct that contains a single specific restriction enzyme recognition site for one or more restriction enzymes, and that serves as the insertion site for a nucleic acid sequence. A multiple cloning site is also referred to as a polylinker or polycloning site. A wide variety of these sites are known in the art.

By “multiple epitope dsRNA” is meant an RNA molecule that has segments derived from multiple target nucleic acids or that has non-contiguous segments from the same target nucleic acid. For example, the multiple epitope dsRNA may have segments derived from (i) sequences representing multiple genes of a single organism; (ii) sequences representing one or more genes from a variety of different organisms; and/or (iii) sequences representing different regions of a particular gene (e.g., one or more sequences from a promoter and one or more sequences from a coding region such as an exon). Desirably, each segment has substantial sequence identity to the corresponding region of a target nucleic acid. In various desirable embodiments, a segment with substantial sequence identity to the target nucleic acid is at least 30, 40, 50, 100, 200, 500, 750, or more nucleotides in length. In desirable embodiments, the multiple epitope dsRNA inhibits the expression of at least 2, 4, 6, 8, 10, 15, 20, or more target genes by at least 20, 40, 60, 80, 90, 95, or 100%. In some embodiments, the multiple epitope dsRNA has non-contiguous segments from the same target gene that may or may not be in the naturally occurring 5′ to 3′ order of the segments, and the dsRNA inhibits the expression of the nucleic acid by at least 50, 100, 200, 500, or 1000% more than a dsRNA with only one of the segments.

By “nucleic acid molecule” is meant a compound in which one or more molecules of phosphoric acid are combined with a carbohydrate (e.g., pentose or hexose) which are in turn combined with bases derived from purine (e.g., adenine or guanine) and from pyrimidine (e.g., thymine, cytosine, or uracil). Particular naturally-occurring nucleic acid molecules include genomic deoxyribonucleic acid (DNA) and genomic ribonucleic acid (RNA), as well as the several different forms of the latter, e.g., messenger RNA (mRNA), transfer RNA (tRNA), and ribosomal RNA (rRNA). Also included are different DNA molecules which are complementary (cDNA) to the different RNA molecules. Synthesized DNA, or a hybrid thereof with naturally-occurring DNA, as well as DNA/RNA hybrids, and PNA molecules (Gambari, Curr Pharm Des 2001 November; 7(17):1839-62) are also included within the definition of “nucleic acid molecule.”

Nucleic acids typically have a sequence of two or more covalently bonded naturally-occurring or modified deoxyribonucleotides or ribonucleotides. Modified nucleic acids include, e.g., peptide nucleic acids and nucleotides with unnatural bases. Modifications include those chemical and structural modifications described under the definition of “dsRNA” below. Also included are, e.g., various structures, as described within the definitions of “dsRNA”, “expression vectors”, and “expression constructs”, and elsewhere in this specification.

By “operably linked” is meant that a gene and one or more transcriptional regulatory sequences, e.g., a promoter or enhancer, are connected in such a way as to permit gene expression when the appropriate molecules (e.g., transcriptional activator proteins) are bound to the regulatory sequences.

By “partial RNA hairpin” is meant a hairpin that has a single-stranded overhang, such as a 5′ or 3′ overhang.

By “phenotype” is meant, for example, any detectable or observable outward physical manifestation, such as molecules, macromolecules, structures, metabolism, energy utilization, tissues, organs, reflexes, and behaviors, as well as anything that is part of the detectable structure, function, or behavior of a cell, tissue, or living organism. Particularly useful in the methods of the invention are dsRNA mediated changes, wherein the detectable phenotype derives from modulation of the function of a cell, modulation of expression of a target nucleic acid, or modulation of the biological activity of a target polypeptide through dsRNA effects on a target nucleic acid molecule.

By “polypeptide biological activity” is meant the ability of a target polypeptide to modulate cell function. The level of polypeptide biological activity may be directly measured using standard assays known in the art. For example, the relative level of polypeptide biological activity may be assessed by measuring the level of the mRNA that encodes the target polypeptide (e.g., by reverse transcription-polymerase chain reaction (RT-PCR) amplification or Northern blot analysis); the level of target polypeptide (e.g., by ELISA or Western blot analysis); the activity of a reporter gene under the transcriptional regulation of a target polypeptide transcriptional regulatory region (e.g., by reporter gene assay, as described below); the specific interaction of a target polypeptide with another molecule, for example, a polypeptide that is activated by the target polypeptide or that inhibits the target polypeptide activity (e.g., by the two-hybrid assay); or the phosphorylation or glycosylation state of the target polypeptide. A compound, such as a dsRNA, that increases the level of the target polypeptide, mRNA encoding the target polypeptide, or reporter gene activity within a cell, a cell extract, or other experimental sample, is a compound that stimulates or increases the biological activity of a target polypeptide. A compound, such as a dsRNA, that decreases the level of the target polypeptide, mRNA encoding the target polypeptide, or reporter gene activity within a cell, a cell extract, or other experimental sample, is a compound that decreases the biological activity of a target polypeptide.

By “promoter” is meant a minimal sequence sufficient to direct transcription of a gene, including PolI, PolII, PolIII, mitochondrial, viral, bacterial, and other promoter sequences that are capable of driving transcription. Also included in this definition are those transcription control elements (e.g., enhancers) that are sufficient to render promoter-dependent gene expression controllable in a cell type-specific, tissue-specific, or temporal-specific manner, or that are inducible by external signals or agents; such elements, which are well-known to skilled artisans, may be found in a 5′ or 3′ region of a gene or within an intron. Desirably a promoter is operably linked to a nucleic acid sequence, for example, a cDNA or a gene in such a way as to permit expression of the nucleic acid sequence.

By “protein” or “polypeptide” or “polypeptide fragment” is meant any chain of more than two amino acids, regardless of post-translational modification (e.g., glycosylation or phosphorylation), constituting all or part of a naturally-occurring polypeptide or peptide, or constituting a non-naturally occurring polypeptide or peptide.

By “reporter gene” or “reporter nucleic acid molecule” is meant any gene that encodes a product whose expression is detectable and/or able to be quantitated by immunological, chemical, biochemical, or biological assays. A reporter gene product may, for example, have one of the following attributes, without restriction: fluorescence (e.g., green fluorescent protein), enzymatic activity (e.g., β-galactosidase, luciferase, chloramphenicol acetyltransferase), toxicity (e.g., ricin A), or an ability to be specifically bound by an additional molecule (e.g., an unlabeled antibody, followed by a labelled secondary antibody, or biotin, or a detectably labelled antibody). It is understood that any engineered variants of reporter genes that are readily available to one skilled in the art, are also included, without restriction, in the foregoing definition.

By “ribonucleic acid complex” or “RNA complex” is meant a chemical association of two or more RNA strands.

By “segment” is meant a fully base-paired RNA molecule (i.e., double-stranded RNA molecule).

By “selective conditions” is meant conditions under which a specific cell or group of cells can undergo selection. For example, the parameters of a fluorescence-activated cell sorter (FACS) can be modulated to identify a specific cell or group of cells. Cell panning, a technique known to those skilled in the art, is another method that employs selective conditions.

By “short dsRNA” or “non-specific dsRNA” is meant a dsRNA as taught in U.S. Ser. No. 60/375,636, filed Apr. 26, 2002, and in U.S. Ser. No. 10/425,006 filed Apr. 28, 2003, “Methods of Silencing Genes Without Inducing Toxicity”, C. Pachuk, both of which are incorporated herein by reference, which can be used to avoid toxicity or an interferon response triggered by long exogenously introduced dsRNA. The short dsRNA has 45, 40, 35, 30, 27, 25, 23, 21, 18, 15, 13, or fewer contiguous nucleotides in length that are in a double-stranded conformation. Unlike an siRNA, the short dsRNA need not have sequence identity to a target polynucleotide, but is used to inhibit or prevent an interferon or RNA stress response normally induced by dsRNA, e.g., dsRNA poly(I)(C). Thus, these methods inhibit the induction of non-specific cytotoxicity and cell death by dsRNA molecules (e.g., exogenously introduced long dsRNA molecules) that would otherwise preclude their use for gene silencing in vertebrate cells and vertebrates. Desirably, the short dsRNA is at least 11 nucleotides in length. In desirable embodiments, the double-stranded region is between 11 to 45, 11 to 40, 11 to 30, 11 to 20, 15 to 20, 15 to 18, 20 to 25, 21 to 23, 25 to 30, or 30 to 40 contiguous nucleotides in length, inclusive. In some embodiments, the short dsRNA is between 30 to 50, 50 to 100, 100 to 200, 200 to 300, 400 to 500, 500 to 700, 700 to 1000, 1000 to 2000, or 2000 to 5000 nucleotides in length, inclusive and has a double-stranded region that is between 11 and 40 contiguous nucleotides in length, inclusive. In one embodiment, the short dsRNA is completely double-stranded. In some embodiments, the short dsRNA is between 11 and 30 nucleotides in length, and the entire dsRNA is double-stranded. In other embodiments, the short dsRNA has one or two single-stranded regions. In particular embodiments, the short dsRNA binds PKR or another protein in a dsRNA-mediated stress response pathway. Desirably, the short dsRNA inhibits the dimerization and activation of PKR by at least 20, 40, 60, 80, 90, or 100%. In some desirable embodiments, the short dsRNA inhibits the binding of a long dsRNA to PKR or another component of a dsRNA-mediated stress response pathway by at least 20, 40, 60, 80, 90, or 100%.

By “specifically hybridizes” is meant a dsRNA that hybridizes to a target nucleic acid molecule but does not substantially hybridize to other nucleic acid molecules in a sample (e.g., a sample from a cell) that naturally includes the target nucleic acid molecule, when assayed under denaturing conditions. In one embodiment, the amount of a target nucleic acid molecule hybridized to, or associated with, the dsRNA, as measured using standard assays, is 2-fold, desirably 5-fold, more desirably 10-fold, and most desirably 50-fold greater than the amount of a control nucleic acid molecule hybridized to, or associated with, the dsRNA.

By “specifically inhibits the expression of a target nucleic acid molecule” is meant that inhibition of the expression of a target nucleic acid molecule in a cell or biological sample occurs to a greater extent than the inhibition of expression of a non-target nucleic acid molecule that has a sequence that is less than 99, 95, 90, 80, or 70% identical or complementary to that of the target nucleic acid molecule. Desirably, the inhibition of expression of the non-target molecule is 2-fold, desirably 5-fold, more desirably 10-fold, and most desirably 50-fold less than the inhibition of expression of the target nucleic acid molecule.

By “substantially pure” is meant a nucleic acid, polypeptide, or other molecule that has been separated from the components that naturally accompany it. Typically, the polypeptide is substantially pure when it is at least 60%, 70%, 80%, 90% 95%, or even 99%, by weight, free from the proteins and naturally-occurring organic molecules with which it is naturally associated. For example, a substantially pure nucleic acid molecule may be obtained by extraction from a natural source, by extraction from a cell that has been genetically engineered to contain the nucleic acid molecule, or by chemical synthesis.

By “substantial sequence complementarity” is meant sufficient sequence complementarity between a dsRNA and a target nucleic acid molecule for the dsRNA to inhibit the expression of the nucleic acid molecule. Preferably, the sequence of the dsRNA is at least 40, 50, 60, 70, 80, 90, 95, or 100% complementary to the sequence of a region of the target nucleic acid molecule.

By “strand” is meant a polymer of ribonucleotides or deoxyribonucleotides, or analogues thereof, that are connected in series by 5′ to 3′ phosphate linkages. The polymer is joined together by a phosphate group, which connects the 5′ carbon of one sugar moiety (ribose, in the case of RNA or deoxyribose, in the case of DNA) of one ribonucleotide or deoxyribonucleotide, respectively, to the 3′ carbon of a second sugar moiety of a second ribonucleotide or deoxyribonucleotide.

By “substantial sequence identity” is meant sufficient sequence identity between a dsRNA and a target nucleic acid molecule for the dsRNA to inhibit the expression of the nucleic acid molecule (e.g., a target gene). Preferably, the sequence of the dsRNA is at least 40, 50, 60, 70, 80, 90, 95, or 100% identical to the sequence of a region of the target nucleic acid molecule. Substantial sequence identity between a ribonucleic acid molecule, e.g., a double-stranded region of an RNA molecule, and a deoxyribonucleic acid molecule, e.g., a target gene, takes into account the presence of a uridine residue in RNA, rather than a thymidine residue, as in DNA. For this reason, an RNA molecule having, for example, the sequence UUUU would be considered 100% identical to a DNA molecule/target gene having the sequence TTTT.

By “sequitope” is meant a contiguous sequence of double-stranded polyribonucleotides that can associate with and activate RISC (RNA-induced silencing complex), usually a contiguous sequence of between 19 and 27 basepairs, e.g., 21 to 23, or 19 to 30 bp, inclusive.

“Multiple-epitope dsRNAs” The advantages of a multiple-epitope or multi-sequitope double-stranded RNA approach as taught in U.S. Ser. No. 60/419,532, filed 18 Oct. 2002, are applicable to utilization of the conserved HBV and/or HCV sequences as taught in U.S. Provisional Application 60/478,076, filed 12 Jun. 2003, “Conserved HBV and HCV Sequences Useful for Gene Silencing”. Because a singular species of dsRNA can simultaneously silence many target genes (e.g., genes from multiple pathogens, multiple genes or sequences from a single pathogen, or genes associated with multiple diseases), a multiple epitope dsRNA can be used for many different indications in the same subject or used for a subset of indications in one subject and another subset of indications in another subject. For such applications, the ability to express long dsRNA molecules (e.g., dsRNA molecules with sequences from multiple genes) without invoking the dsRNA stress response is highly desirable. For example, by using a series of sequences, each, e.g., as short as 19-21 nucleotides, desirably 100 to 600 nucleotides, or easily up to 1, 2, 3, 4, 5, or more kilobases such that the total length of such sequences is within the maximum capacity of the selected plasmid (e.g., 20 kilobases in length), a single such pharmaceutical composition can provide protection against a large number of pathogens and/or toxins at a relatively low cost and low toxicity, e.g., HBV, HCV, HIV, etc. The use of dsRNAs having multiple double-stranded regions separated by single-stranded regions as taught in the instant invention is particularly amenable to such applications. The double-stranded regions can include a single sequitope which does not require an enzyme such as Dicer for activation, or can include longer regions having multiple-sequitopes which require Dicer for cleavage into double-stranded units of the appropriate length.

The use of multiple epitopes derived from one or more genes from multiple strains and/or variants of a highly variable or rapidly mutating pathogen such as HBV and/or HCV can also be very advantageous. For example, a singular dsRNA species that recognizes and targets multiple strains and/or variants of HBV and/or HCV can be used as a universal treatment or vaccine for the various strains/variants of influenza.

The ability to silence multiple genes of a particular pathogen, e.g., HBV and/or HCV prevents the selection of HBV and/or HCV “escape mutants.” In contrast, typical small molecule treatment or vaccine therapy that only targets one gene or protein results in the selection of pathogens that have sustained mutations in the target gene or protein and the pathogen thus becomes resistant to the therapy. By simultaneously targeting a number of genes or sequences of the pathogen and or extensive regions of the pathogen using the multiple epitope approach of the present invention, the emergence of such “escape mutants” is effectively precluded.

By “target”, “target nucleic acid”, “target gene”, “target polynucleotide” or “target polynucleotide sequence” is meant any nucleic acid sequence present in a eukaryotic cell, plant or animal, vertebrate or invertebrate, mammalian, avian, etc., whether a naturally-occurring, and possibly defective, polynucleotide sequence, or a heterologous sequence present due to an intracellular or extracellular pathogenic infection or a disease, whose expression is modulated as a result of post-transcriptional gene silencing, transcriptional gene silencing, or other sequence-specific dsRNA-mediated inhibition. As used herein, the “target”, “target nucleic acid”, “target gene”, or “target polynucleotide sequence” may be in the cell in which the PTGS, transcriptional gene silencing (TGS), or other gene silencing event occurs, or it may be in a neighboring cell, or in a cell contacted with media or other extracellular fluid in which the cell that has undergone the PTGS, TGS, or other gene silencing event is contained. Such a “target”, “target nucleic acid”, “target gene”, or “target polynucleotide sequence” may be a coding sequence, that is, it is transcribed into an RNA, including an mRNA, whether or not it is translated to express a protein or a functional fragment thereof. Alternatively, it may be non-coding, but may have a regulatory function, including a promoter, enhancer, repressor, or any other regulatory element. The term “gene” is intended to include any target sequence intended to be “silenced”, whether or not transcribed and/or translated, including regulatory sequences, such as promoters.

Exemplary “target”, “target nucleic acid”, “target gene”, or “target polynucleotide sequence” molecules include nucleic acid molecules associated with cancer or abnormal cell growth, such as oncogenes, and nucleic acid molecules associated with an autosomal dominant or recessive disorder (see, for example, WO 00/63364, WO 00/44914, and WO 99/32619). Desirably, the dsRNA inhibits the expression of an allele of a nucleic acid molecule that has a mutation associated with a dominant disorder and does not substantially inhibit the other allele of the nucleic acid molecule (e.g., an allele without a mutation associated with the disorder). Other exemplary “target”, “target nucleic acid”, “target gene”, or “target polynucleotide sequence” molecules include host cellular nucleic acid molecules and pathogen nucleic acid molecules including coding and non-coding regions required for the infection or propagation of a pathogen, such as a virus, bacteria, yeast, fungus, protozoa, or parasite.

By “target polypeptide” is meant a polypeptide whose biological activity is modulated as a result of gene silencing. As used herein, the target polypeptide may be in the cell in which the PTGS, TGS, or other gene silencing event occurs, or it may be in a neighboring cell, or in a cell contacted with media or other extracellular fluid in which the cell that has undergone the PTGS, TGS, or other gene silencing event is contained.

By “transformation” or “transfection” is meant any method for introducing foreign molecules into a cell (e.g., a bacterial, yeast, fungal, algal, plant, insect, or animal cell, particularly a vertebrate or mammalian cell). The cell may be in an animal. Lipofection, DEAE-dextran-mediated transfection, microinjection, protoplast fusion, calcium phosphate precipitation, viral or retroviral delivery, electroporation, and biolistic transformation are just a few of the transformation/transfection methods known to those skilled in the art. The RNA or RNA expression vector (DNA) may be naked RNA or DNA or local anesthetic complexed RNA or DNA (Pachuk et al., supra). Other standard transformation/transfection methods and other RNA and/or DNA delivery agents (e.g., a cationic lipid, liposome, or bupivacaine) are described in WO 00/63364, filed Apr. 19, 2000 (see, for example, pages 18-26). The dsRNAs or dsRNA expression constructs may also be complexed with the multifunctional molecular complexes of U.S. Pat. No. 5,837,533, U.S. Pat. No. 6,127,170, or U.S. Pat. No. 6,379,965 (Boutin), or the multifunctional molecular complexes or oil/water cationic amphiphile emulsions of PCT/US03/14288, filed May 6, 2003 (Satishchandran). Commercially available kits can also be used to deliver RNA or DNA to a cell. For example, the Transmessenger Kit from Qiagen, an RNA kit from Xeragon Inc., and an RNA kit from DNA Engine Inc. (Seattle, Wash.) can be used to introduce single or dsRNA into a cell.

By “transformed cell” or “transfected cell” is meant a cell (or a descendent of a cell) into which a nucleic acid molecule, for example, a dsRNA or double-stranded expression vector has been introduced, by means of recombinant nucleic acid techniques. Such cells may be either stably or transiently transfected.

By “treating, stabilizing, or preventing cancer” is meant causing a reduction in the size of a tumor, slowing or preventing an increase in the size of a tumor, increasing the disease-free survival time between the disappearance of a tumor and its reappearance, preventing an initial or subsequent occurrence of a tumor, or reducing or stabilizing an adverse symptom associated with a tumor. In one embodiment, the percent of cancerous cells surviving the treatment is at least 20, 40, 60, 80, or 100% lower than the initial number of cancerous cells, as measured using any standard assay. Preferably, the decrease in the number of cancerous cells induced by administration of a composition of the invention is at least 2, 5, 10, 20, or 50-fold greater than the decrease in the number of non-cancerous cells. In yet another embodiment, the number of cancerous cells present after administration of a composition of the invention is at least 2, 5, 10, 20, or 50-fold lower than the number of cancerous cells present after administration of a vehicle control. Preferably, the methods of the present invention result in a decrease of 20, 40, 60, 80, or 100% in the size of a tumor as determined using standard methods. Preferably, at least 20, 40, 60, 80, 90, or 95% of the treated subjects have a complete remission in which all evidence of the cancer disappears. Preferably, the cancer does not reappear, or reappears after at least 5, 10, 15, or 20 years. In another desirable embodiment, the length of time a patient survives after being diagnosed with cancer and treated with a composition of the invention is at least 20, 40, 60, 80, 100, 200, or even 500% greater than (i) the average amount of time an untreated patient survives or (ii) the average amount of time a patient treated with another therapy survives.

By “treating, stabilizing, or preventing a disease or disorder” is meant preventing or delaying an initial or subsequent occurrence of a disease or disorder; increasing the disease-free survival time between the disappearance of a condition and its reoccurrence; stabilizing or reducing an adverse symptom associated with a condition; or inhibiting or stabilizing the progression of a condition. This includes prophylactic treatment, in which treatment before infection with an infectious agent, such as a virus, bacterium, or fungus, is established, prevents or reduces the severity or duration of infection. Preferably, at least 20, 40, 60, 80, 90, or 95% of the treated subjects have a complete remission in which all evidence of the disease disappears. In another embodiment, the length of time a patient survives after being diagnosed with a condition and treated using a method of the invention is at least 20, 40, 60, 80, 100, 200, or even 500% greater than (i) the average amount of time an untreated patient survives, or (ii) the average amount of time a patient treated with another therapy survives.

By “under conditions that inhibit or prevent an interferon response or a dsRNA stress response” is meant conditions that prevent or inhibit one or more interferon responses or cellular RNA stress responses involving cell toxicity, cell death, an anti-proliferative response, or a decreased ability of a dsRNA to carry out a PTGS or TGS event. These responses include, but are not limited to, interferon induction (both Type 1 and Type II), induction of one or more interferon stimulated genes, PKR activation, 2′5′-OAS activation, and any downstream cellular and/or organismal sequalae that result from the activation/induction of one or more of these responses. By “organismal sequalae” is meant any effect(s) in a whole animal, organ, or more locally (e.g., at a site of injection) caused by the stress response. Exemplary manifestations include elevated cytokine production, local inflammation, and necrosis. Desirably the conditions that inhibit these responses are such that not more than 95%, 90%, 80%, 75%, 60%, 40%, or 25%, and most desirably not more than 10% of the cells undergo cell toxicity, cell death, or a decreased ability to carry out a PTGS, TGS, or another gene silencing event, compared to a cell not exposed to such interferon response inhibiting conditions, all other conditions being equal (e.g., same cell type, same transformation with the same dsRNA expression library.

Apoptosis, interferon induction, 2′5′ OAS activation/induction, PKR induction/activation, anti-proliferative responses, and cytopathic effects are all indicators for the RNA stress response pathway. Exemplary assays that can be used to measure the induction of an RNA stress response as described herein include a TUNEL assay to detect apoptotic cells, ELISA assays to detect the induction of alpha, beta and gamma interferon, ribosomal RNA fragmentation analysis to detect activation of 2′5′OAS, measurement of phosphorylated eIF2a as an indicator of PKR (protein kinase RNA inducible) activation, proliferation assays to detect changes in cellular proliferation, and microscopic analysis of cells to identify cellular cytopathic effects. Desirably, the level of an interferon response or a dsRNA stress response in a cell transformed with a dsRNA or a dsRNA expression vector is less than 20, 10, 5, or 2-fold greater than the corresponding level in a mock-transfected control cell under the same conditions, as measured using one of the assays described herein. In other embodiments, the level of an interferon response or a dsRNA stress response in a cell transformed with a dsRNA or a dsRNA expression vector using the methods of the present invention is less than 500%, 200%, 100%, 50%, 25%, or 10% greater than the corresponding level in a corresponding transformed cell that is not exposed to such interferon response inhibiting conditions, all other conditions being equal. Desirably, the dsRNA does not induce a global inhibition of cellular transcription or translation.

By “viral infection” is meant the invasion of a host animal by a virus. For example, the infection may include the excessive growth of viruses that are normally present in or on the body of an animal or growth of viruses that are not normally present in or on the animal. More generally, a viral infection can be any situation in which the presence of a viral population(s) is damaging to a host animal. Thus, an animal is “suffering” from a viral infection when an excessive amount of a viral population is present in or on the animal's body, or when the presence of a viral population(s) is damaging the cells or other tissue of the animal.

Conditions and techniques that can be used to prevent an interferon response or dsRNA stress response during the methods of the present invention are described herein.

### BRIEF DESCRIPTION OF THE DRAWINGS

FIG. 1A is an illustration of duplex dsRNAs with double-stranded (ds) regions punctuated by mismatched regions in which basepairing does not occur. FIG. 1A contains a structure sometimes referred to as a dumbbell structure. The mismatched or non-basepaired regions appear as “bubbles” between the basepaired regions of dsRNA. The sizes of the double-stranded regions and loops or mismatched regions are as described elsewhere herein.

FIG. 1B is an illustration showing cleavage (processing) of the single-stranded “bubble” mismatched regions of FIG. 1A. Single-strand specific ribonucleases (ssRNases) cleave the single-stranded mismatched regions into smaller dsRNA duplexes.

FIG. 1C is an illustration that shows that the number of ribonucleotides in a first and a second RNA strand that form a mismatched region of an RNA complex does not have to be of the same length, and in fact, the number of ribonucleotides that can differ between the two strands that form a mismatch region can be as few as one (e.g., a one nucleotide insertion or deletion in a single RNA strand). The result of a one nucleotide mismatch is a 4 nucleotide “bubble” of non-basepaired nucleotides because of steric constraints on basepairing.

FIG. 2A is an illustration of a hairpin dsRNA molecule that contains multiple double-stranded (ds) regions punctuated by mismatched regions in which basepairing does not occur. The mismatched or non-basepaired regions appear as a terminal “loop”, in the case of the hairpin/stem-loop structure, or as “bubbles” between the basepaired regions of the dsRNA molecule. The size of the ds region and the loop or “bubble” mismatched region varies, as is described elsewhere herein.

FIG. 2B is an illustration demonstrating cleavage (processing) of the single-stranded “bubble” mismatched regions and the “loop” region of a dsRNA molecule by single-strand specific ribonucleases (RNAses), which yields smaller dsRNA duplexes.

FIG. 2C is an illustration showing that the number of nucleotides in the 5′ strand of a mismatched region of a dsRNA hairpin molecule does not have to be the same as the number of nucleotides in the 3′ strand of a mismatched region of a dsRNA molecule, and in fact, the number of ribonucleotides that can differ between the two strands that form the mismatch region can be as few as one (e.g., a one nucleotide insertion or deletion in a single RNA strand). The result of a one nucleotide mismatch is a 4 nucleotide “bubble” of non-basepaired nucleotides because of steric constraints on basepairing.

FIG. 3A is an illustration showing a structured RNA molecule containing a series of hairpin regions interspersed by single-stranded spacer regions (e.g., mismatched or unpaired regions). Each hairpin region is comprised of a double-stranded “stem” region and a single-stranded “loop” region.

FIG. 3B is an illustration showing cleavage processing) of the single-stranded loop and spacer regions by single-strand specific RNAses, thereby yielding dsRNA duplexes, e.g., short dsRNA duplexes.

FIG. 4A is an illustration showing two separate plasmids (plasmid A and plasmid B). Plasmid A contains Cistron #1 under the control of a T7 promoter, while plasmid B contains Cistron #2 under the control of a T7 promoter. Cistron #1 of plasmid A encodes one RNA strand, Strand A, while Cistron #2 of plasmid B encodes one strand, Strand B. Transcription of Strand A from plasmid A and Strand B from plasmid B yields two RNA molecules that can hybridize together to form a duplex RNA complex containing two mismatched regions. As shown, transcription of each cistron within the same cell enables Strand A to anneal with Strand B to form a duplex RNA containing double-stranded regions interspersed by mismatched regions.

FIG. 4B is an illustration showing that the cistrons of FIG. 4A can be located within the same expression vector, e.g., separate plasmids can encode the two RNA strands, as indicated in FIG. 4A, or the RNA strands can be encoded by the same plasmid, as depicted in FIG. 4B. As shown, transcription of each cistron within the same cell enables Strand A to anneal with Strand B to form a duplex RNA containing double-stranded regions interspersed by mismatched regions.

FIG. 5A is an illustration showing the construction of a vector construct encoding the sense strand of a large RNA duplex punctuated with mismatched regions. In FIG. 5A, Section 1, three DNA oligonucleotide pairs are depicted: oligo 1, oligo 2, and oligo 3. Each pair is comprised of a top strand and a complementary bottom strand. Box A of each oligonucleotide is comprised of a sequence of at least 19 nucleotides derived from 19 contiguous nucleotides of a target nucleic acid sequence. The top strand Box A of each oligonucleotide encodes a sequence that is the same polarity as the RNA target nucleic acid sequence, while the bottom strand Box A encodes the complement to that sequence. The top strand is designed to be transcribed. The target nucleic acid sequence provided in Box A of oligo 1, 2, or 3 could be the same or different. Box B represents those sequences that are designed to be mismatched with the antisense strand of the large dsRNA duplex (not to be confused with the bottom strand of the oligonucleotide pair). Box B sequences can be any sequence provided that it does not basepair with Box B sequences in the antisense strand of the large dsRNA duplex. In this particular example, the Box B sequences that will be present on the transcribed strand are T residues. In FIG. 5A, Section 2, the top strand of each oligo pair is annealed to its complement, the bottom strand, generating a double-stranded DNA oligonucleotide. In FIG. 5A, Section 3, the annealed oligonucleotide pairs are directionally ligated (as taught in U.S. Pat. No. 6,143,527, “Chain reaction cloning using a bridging oligonucleotide and DNA ligase”, Pachuk, C., Samuel, M., and Satishchandran, C., incorporated herein by reference.) such that oligo 1 is ligated to oligo 2 which is ligated to oligo 3 in the polarity indicated in the figure. The ligation product can be amplified through PCR using primers that are situated at each end of the ligation product. In FIG. 5A, Section 4, the ligation product or amplified product is directionally ligated into a vector as shown such that the top strand (sense polarity with respect to the target RNA) is transcribed. As shown in FIG. 5A, Section 4, transcription results in a sense strand RNA that is of the same polarity as the top strands of the oligonucleotide used during synthesis of the construct.

FIG. 5B is an illustration showing the construction of a vector construct encoding the antisense strand of a large RNA duplex with mismatched regions. In FIG. 5B, Section 1, three DNA oligonucleotide pairs are depicted: oligo 4, oligo 5 and oligo 6. Each pair is comprised of a top strand and a complementary bottom strand. Box A of each oligonucleotide is comprised of at least 19 nucleotides derived from a sequence of at least 19 contiguous nucleotides of a target RNA sequence. The top strand Box A encodes sequences that are the same polarity as the RNA target nucleic acid sequence while the bottom strand Box A encodes the complement to those sequences. The bottom strand is designed to be transcribed. Transcription of the bottom strand generates an antisense RNA with respect to the target RNA. Box B represents those sequences that are designed to be mismatched with the sense strand of the large dsRNA duplex (not to be confused with the top strand of the oligonucleotide pair). Box B sequences can be any sequence providing that it does not basepair with Box B sequences in the sense strand of the large dsRNA duplex. In this particular example, the Box B sequences that will be present on the antisense strand are G residues. In FIG. 5B, Section 2, the top strand of each oligo pair is annealed to its complement, the bottom strand, generating a ds DNA oligonucleotide. In FIG. 5B, Section 3, the annealed oligonucleotide pairs are directionally ligated (as taught in U.S. Pat. No. 6,143,527, “Chain reaction cloning using a bridging oligonucleotide and DNA ligase”, Pachuk, C., Samuel, M., and Satishchandran, C., incorporated herein by reference) such that oligo 4 is ligated to oligo 5 which is ligated to oligo 6 in the polarity indicated in the figure. The ligation product can be amplified through PCR using primers that are situated at each end of the ligation product. In FIG. 5B, Section 4, the ligation product or amplified product is directionally ligated into a vector, e.g., a plasmid as shown, such that the bottom strand (antisense polarity with respect to the target RNA) is transcribed. Transcription, e.g., from the T7 bacteriophage promoter, results in an antisense strand RNA that is of the same polarity as the bottom strands of the oligonucleotides 4, 5 and 6 used during synthesis of the construct. As shown in FIG. 5B, Section 5, transcription of both the sense strand of FIG. 5A, Section 4, and the antisense strand of FIG. 5B, Section 4, in the same cell results in annealing of both strands generating a duplex dsRNA containing mismatched regions, as indicated.

FIG. 6 is an illustration showing the construction of a vector construct encoding an RNA hairpin with double-stranded regions interspersed with mismatched regions. In FIG. 6, Section 1, six DNA oligonucleotide pairs are shown, oligo pairs 1-6. Each pair is comprised of a top strand and a bottom strand. Box A of each oligonucleotide is comprised of at least 19 nucleotides derived from a sequence of at least 19 contiguous nucleotides of a target RNA sequence. For illustrative purposes, six contiguous nucleotides are depicted for each Box A. The “ . . . ” denotes the remaining sequences of Box A that are not shown. In this particular example, the top strands of oligopairs 1, 2, and 3 are the same polarity as the target RNA, while the top strands in oligo pairs 4, 5, and 6 represent the antisense polarity with respect to the target sequence. The top strands of oligopairs 4, 5, and 6 encode the antisense sequence with respect to the top strands of oligopairs 3, 2, and 1, respectively. In FIG. 6, Section 2, following annealing of the top and bottom strands of each oligopair, the annealed oligos are directionally ligated according to the methods of U.S. Pat. No. 6,143,527, to yield a sequence of oligo 1, oligo 2, oligo 3, oligo 4, oligo 5, oligo 6, as indicated. This sequence can first be PCR amplified or directly ligated into a vector of choice, e.g., a plasmid as shown is FIG. 6, Section 3, the ligation product can first be PCR amplified or directly ligated into a vector of choice. The product can be ligated into the vector in any orientation with respect to the promoter, e.g., the bacteriophage T7 promoter. Transcription of the insert yields a hairpin RNA with double-stranded regions interspersed with single-stranded mismatched regions including a single-stranded “loop” between oligo 3 and oligo 4, as shown in FIG. 6, Section 4.

FIG. 7 is an illustration showing the construction of a vector construct encoding an “udderly” structured RNA, comprising a plurality of hairpin or stem-loop RNAs interspersed by single-stranded “spacer” regions (e.g., mismatched or unpaired regions). In FIG. 7, Section 1, two dsDNA oligonucleotides, oligo A and oligo B, encoding short hairpin loops are depicted. Converging arrows represent inverted repeat or sense (“S”) and antisense (“AS”) sequences flanking a “loop” sequence. Extra sequences (“spacer”) are encoded at the ends of each oligo to serve as spacer elements. The structure of the encoded hairpin-loop RNA is shown below the oligonucleotide. The structure of each RNA is composed of two spacer elements, located at the termini of the RNA molecule, and a hairpin, comprising a double-stranded stem region (“stem”) and a single-stranded loop region (“loop”). One strand of the stem region is composed of between 19 and 30 nucleotides derived from between 19 and 30 contiguous nucleotides of a target nucleic acid sequence and the other strand of the stem region is complementary to this strand. The loop may be 1 to about 100 nucleotides, about 11 to 100 nucleotides, or desirably, about 4-10 nucleotides in length. There can be some degree of mismatch tolerated between the partner strands when the double-stranded stem region is greater than 19 nucleotides in length. In general, however, at least 19 contiguous nucleotides of one hairpin must be able to basepair with its complementary partner hairpin strand. In FIG. 7, Section 2, the oligos are ligated to generate ligation products, some of which contain oligo A juxtaposed to oligo B as depicted. The ligation product (or PCR amplified product) is ligated into a vector of choice. Transcription of the insert results in an RNA molecule having the structure depicted in FIG. 7, Section 3. For this type of multiple hairpin RNA structure, the minimal number of oligos used is 2 and the maximum number is desirably 500, with a desirable range of 2 to 100. The dsDNA oligos represented in a ligation product may all be unique; they may all be identical, or they may be any combination of the same or different sequences.

FIG. 8A illustrates a plasmid (A-1), described in more detail in Example 9, which is designed to contain HBV sequences in tandem, in the antisense and sense orientations separated by the loop sequence. HBV sequence is designated as, A-B-C-D-(loop)-D′-C′-B′-A′. The A-B-C-D region and the D′-C′-B′-A′ region is each between 19 and 27 nucleotides, as indicated. The arrangement results in transcription of an RNA molecule that folds back on itself to form a stem-loop structure. The promoter at the S′ end is the RNA polymerase III promoter U6. At the 5′ end, a flanking sequence is added that includes multiple G residues. The major transcription site of the plasmid is indicated (by the arrow) 5′ to the HBV sequence beginning at A. The several G residues are included to force transcription initiation if the major transcription start site is missed; these are referred to as minor transcription start sites. Similarly, at the 3′ end of the HBV sequence (A′), a flanking sequence with one or more terminators as described above is provided. Various transcripts will terminate at different sites in the 3′ flanking sequence. A large majority are predicted to terminate at the Major Termination Site. In this particular embodiment, the 5′ and the 3′ flanking sequences are designed not to hybridize with each other or with the HBV sequences. Following transcription, four different types of RNA molecules (designated I, II, III, and IV) can be generated due to staggered initiation and termination sites. These RNA molecules fold into stem-loop structures with varying single-stranded 5′ and 3′ ends, as shown. (Only extreme examples of transcripts and structures are shown.) These molecules will all be processed by single-strand cellular RNAases to yield a siRNA molecule of 19 to 27 bp, as shown.

FIG. 8B is an illustration of a plasmid (A-2) designed to contain HBV sequences in tandem, in the antisense and sense orientations separated by the loop sequence. HBV sequence is designated as A-B-C-D-(loop)-D′-C′-B′-A′. The A-B-C-D region and the D′-C′-B′-A′ region is each between 19 and 27 nucleotides as indicated. The arrangement results in transcription of an RNA molecule that folds back on itself to form a stem-loop structure. The promoter at the 5′ end is the RNA polymerase III promoter U6. At the 5′ end, a flanking sequence is provided with multiple G residues. The major transcription site is indicated (by the arrow) 5′ to the HBV sequence beginning at A. Several G residues are included to force transcription initiation if the major transcription start site is missed; these are referred to as minor transcription start sites. Similarly, at the 3′ end of the HBV sequence (A′), a set of flanking sequences are provided to force termination. Various transcripts will terminate at different sites in the 3′ flanking sequence. A large majority of the transcripts are predicted to terminate at the Major Termination Site. In this particular embodiment, the 5′ and the 3′ flanking sequences are designed to hybridize with each other, but not to the HBV sequences. Following transcription, four different types of RNA molecules (designated I, II, III, and IV) can be generated. These RNA molecules fold into the structures shown. (Only extreme examples of transcripts and structures are shown.) As can be seen, these molecules will be processed by single-strand cellular RNAases ((II, III, and IV), or by both single-strand cellular RNAases and Dicer (I), to yield siRNA molecules of 19 to 27 base pairs.

FIG. 8C illustrates two plasmids (B-1 and C-1) designed to contain HBV sequences. One (B-1) contains the antisense sequence A-B-C-D and the other (C-1) contains the sense sequence D′-C′-B′-A′ and thus, are in opposite orientations with respect to the promoter. The A-B-C-D region and the D′-C′-B′-A′ region is each between 19 and 27 nucleotides, as indicated. When the two plasmids are co-transfected into a cell, the two transcripts will hybridize to each other to form a duplex double-stranded structure, as shown. The promoter at the 5′ end is the RNA polymerase III promoter U6. A 5′ flanking sequence is provided with multiple G residues, as described herein. The major transcription site of each plasmid is indicated (by the arrow) 5′ to the HBV sequences beginning at A in one plasmid and at D′ in the other. Several G residues are included to force transcription initiation if the major transcription start site is missed; these are referred to as minor transcription start sites. Similarly, at the 3′ end of the HBV sequences (D and A′) flanking sequences with terminators are provided. Various transcripts will terminate at different sites in the 3′ flanking sequence. A large majority of the transcripts are predicted to terminate at the Major Termination Site. In this embodiment, the 5′ and the 3′ flanking sequences are designed not to hybridize with each other, or with the HBV sequences. Following transcription, four different types of RNA molecules (I, II, III, and IV) can be generated. These fold into the structures shown. (Only extreme examples of transcripts and structures are shown.) These molecules will be processed by cellular single-strand RNAases to result in siRNA molecules of 19 to 27 basepairs.

FIG. 8D illustrates two plasmids (B-2 and C-2) designed to contain HBV sequences. One (B-2) contains the antisense sequence A-B-C-D and the other (C-2) contains the sense sequence D′-C′-B′-A′ and thus are in opposite orientations to the promoter. The A-B-C-D sequence and the D′-C′-B′-A′ sequence are each 19 to 27 nts in length as indicated. When the two plasmids are co-transfected into a cell, the RNA transcripts will hybridize to each other to form a duplex double-stranded RNA structure as shown. The promoter at the 5′ end is the RNA polymerase III promoter U6. A 5′ flanking sequence as described herein is provided with multiple G residues to force initiation of transcription. The major transcription site is indicated (by arrow) 5′ to the HBV sequences beginning at A in plasmid B-2 and at D′ in C-2. Several G residues are included to force transcription initiation if the major transcription start site is missed; these are referred to as minor transcription start sites. Similarly, at the 3′ end of the HBV sequences (after D and A′, respectively) a flanking sequence with one or more terminators is provided. Various transcripts will terminate at various such terminator sites in the 3′ flanking sequence. A large majority are predicted to terminate at the Major Termination Site. In this embodiment, the 5′ flanking sequences and the 3′ flanking sequences are designed to hybridize with each other as shown, but not to the HBV sequences. Following transcription, four different types of RNA molecules (I, II, III, and IV) can be generated. These RNA molecules fold into the structures shown. Only extreme examples of transcripts and structures are shown. These molecules will be processed by either Dicer or cellular RNAases, or both, to result in siRNA molecules of the requisite 19 to 27 basepairs.

FIG. 8E is an illustration showing various substructures of two RNA molecules (I and II) that can be transcribed to assist in the folding and formation of RNA structures that are readily processed to yield the siRNA molecules that are potent initiators of RNAi. The structure designated as A-B-C-D-(loop)-D′-C′-B′-A′ represents the HBV sequences in two opposing orientations, either sense followed by antisense or vice versa, as described above; the additional single-stranded loops and double-stranded stems beyond those described above are intended to more readily generate the desired shRNA-like stem-loop structures, e.g., by encouraging neighboring nucleotide sequences to participate in certain interactions thereby minimizing unwanted basepairing. In addition, the basepairing shown between the 5′ and 3′ flanking regions results in a more stable RNA molecule that is resistant to exonucleases.

FIG. 8F is an illustration of a dsRNA molecule containing many embodiments of the present invention. The dsRNA molecule of FIG. 8F contains, at the 5′ end, a 5′ stabilizing short stem-loop sequence, as described in Example 10 (a “Bernie Moss” hairpin), followed by Dicer dependent and Dicer independent dsRNA structures containing A-B-C (loop)-C′-B′-A′ as HBV specific sequences. The dsRNA molecule is designed to fold into stem-loop structures that contain more than a sequitope length (>19-30 basepairs) of siRNA, but specific to HBV. These structures will be processed by the enzyme Dicer. A combination of these substructures of RNA assist in RNA folding and aid in the formation of structures that when transcribed are readily processed to yield siRNA molecules that are potent initiators of RNAi. The additional loops and stems beyond those described above are intended to generate the desired shRNA-like stem-loop structures readily, by minimizing unwanted basepairing through engaging the neighboring sequences to participate in other interactions. In addition, basepairing of the flanking regions results in a more stable RNA molecule that is resistant to exonucleases. Furthermore, two distant sequences of the RNA molecule fold back to form additional stem-like structures that may be processed in either Dicer-dependent or Dicer-independent manners. The methods taught in this application may be used to construct a dsRNA molecule comprising the multiple long and/or short hairpin structures depicted in FIG. 8F, which comprise strings of stem-loop or hairpin structures interspersed by double-stranded regions. Some of the stem-loop or hairpins are designed to enhance stability by preventing from degradation (cleavage) by exonucleases. For example, as seen in FIG. 8F, a stem-loop structure located in the 5′-most portion of the RNA molecule, e.g., a stability enhancing Bernie Moss hairpin, as described in more detail in Example 10, and as depicted in FIG. 9, may serve to protect the transcript, including downstream effector portions of the molecule, from degradation. The construct of FIG. 5F also includes a 5′ initiation sequence, as described in Example 9. The dsRNA constructs may be “Dicer independent”, e.g., the double-stranded stem regions may be about 19 to about 30 basepairs in length, such that cleavage of the single-stranded regions by single-strand cellular RNAases yields dsRNAs of 19 to 30 bp, without any cleavage by Dicer or similar enzymes, which cleave dsRNA greater than 19-30 basepairs in length. Such siRNAs (short interfering RNAs) or “sequitopes” are contiguous sequences of double-stranded polyribonucleotides that can associate with and activate RISC (RNA-induced silencing complex), usually a contiguous sequence of between 19 and 27 basepairs, e.g., 21 to 23, or 19 to 30 bp, inclusive. The dsRNA constructs may also be “Dicer-dependent”, e.g., the double-stranded stem regions may be greater than about 27 to 30 basepairs in length, so that cleavage of the single-stranded regions by single-strand RNAases yields dsRNAs of greater than about 27 to about 30 basepairs, so that further dsRNA cleavage by Dicer or similar enzymes is necessary for formation of siRNAs of 19-30 basepairs that are capable of associating with, and activating, the RISC complex. As shown in FIG. 8F, the sequences separating the stem-loop structures may be double-stranded. The “shoulder” regions comprising the several nucleotides between the stem-loop structures and the double-stranded separating regions will include a region of at least about four nucleotides, more if so desired, that will be single-stranded and will be amenable to cleavage by single-strand RNAases. If the double-stranded separating sequences comprise regions of substantial sequence homology to a target polynucleotide, e.g., at least 19 to 30 contiguous basepairs (desirably, no greater than about 200 basepairs, preferably, no greater than about 50 basepairs), they can also be cleaved to produce additional dsRNAs capable of inducing inhibition or silencing of a target. As seen in FIG. 8F, a single such structure can easily be engineered to include both Dicer-dependent and Dicer-independent double-stranded regions.

FIGS. 8A-8F are described in more detail in Example 9.

FIG. 9 is an illustration showing the secondary structure of an RNA transcript encoded by the expression construct described in Example 10. At the 5′ terminus is the “BM” hairpin, followed by a linker or spacer region, selected in this example to lack homology to any known human genomic sequences, followed by a dsRNA “Effector” hairpin. Providing the 5′ “BM” hairpin-linker region provides transcript stability and protects the sequences of the effector portion of the molecule from degradation. The effector portion of the molecule could be any dsRNA molecule capable of inducing dsRNA-mediated silencing, including expressed or synthesized, duplex or hairpin, long or short, including the many types of structured dsRNA, such as double-stranded RNA sequences separated by mismatched regions, multiple hairpin constructs, udderly-structured, and/or partial and/or forced hairpins, including Dicer-dependent and/or Dicer-independent structures.

### DETAILED DESCRIPTION

We have previously reported that induction of an undesired interferon response and activation of the various components comprising this response is mediated by the particular dsRNA delivery/expression method used. Importantly, not all methods of dsRNA presentation activate this response (see, e.g., U.S. Ser. Nos. 60/378,191, filed May 6, 2002; 60/375,636; filed Apr. 26, 2002; 10/062,707, filed Jan. 31, 2002; U.S. Published Application 2002/0132257 and European Published Application EP 1229134 which are each hereby incorporated by reference).

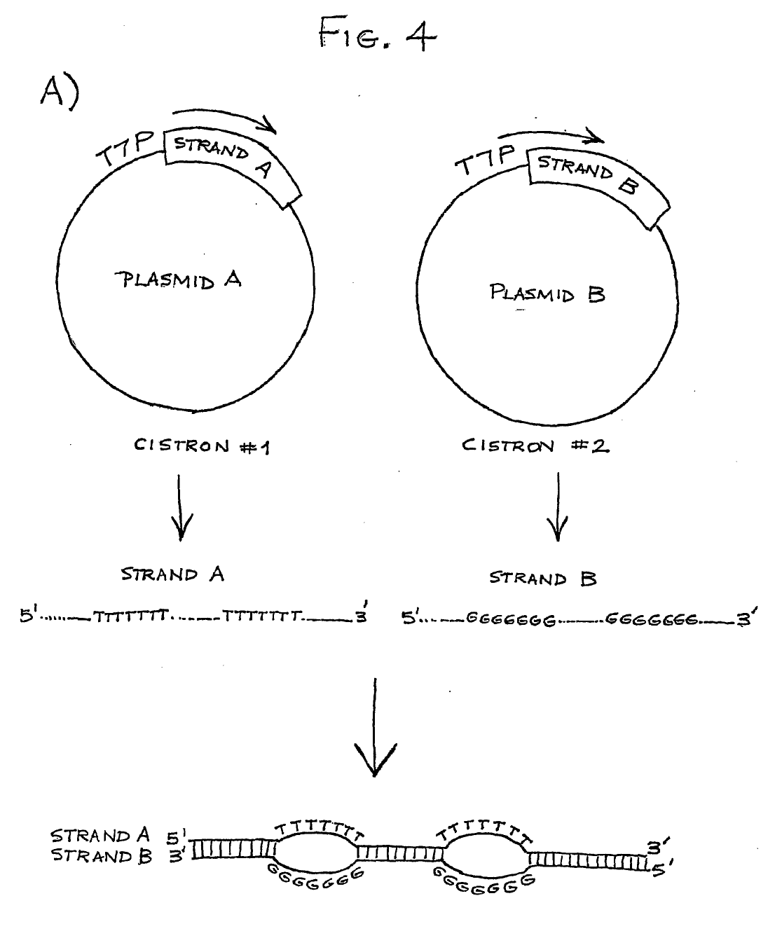

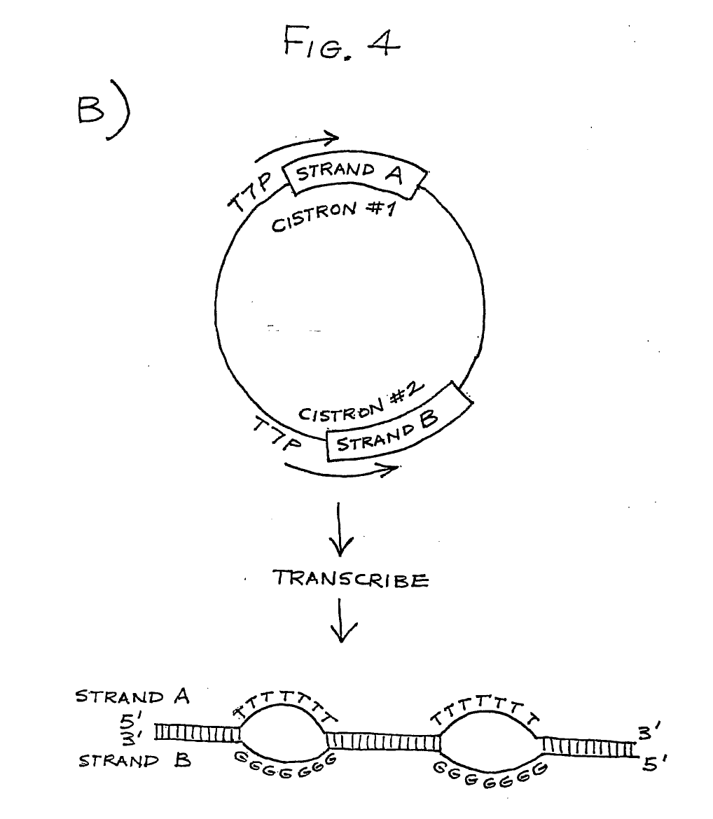

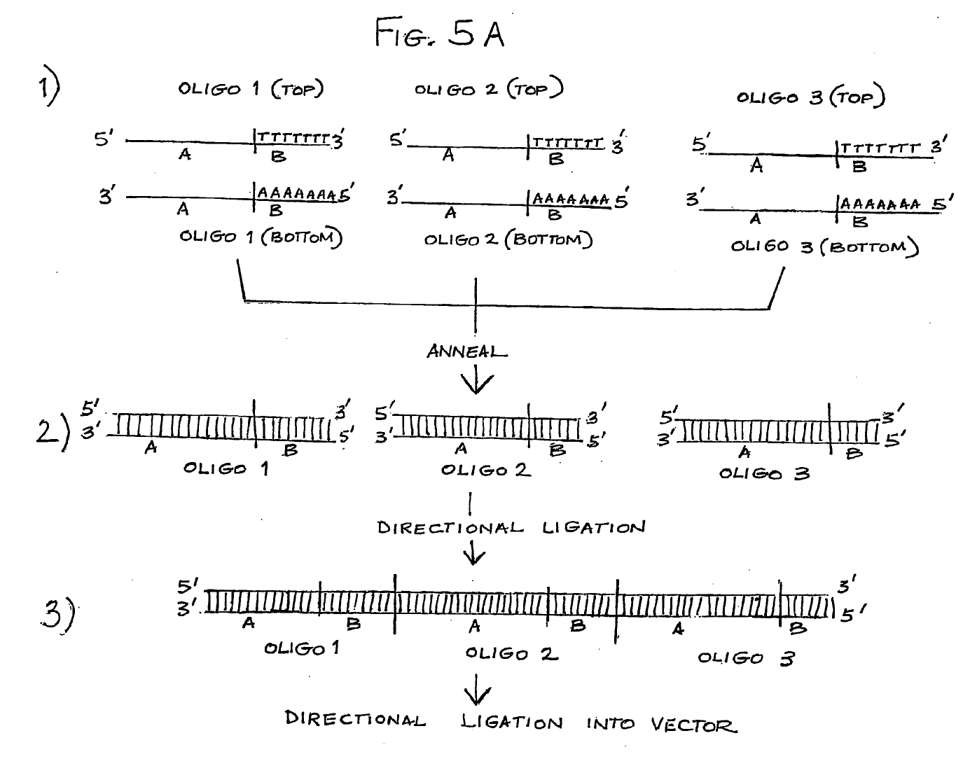

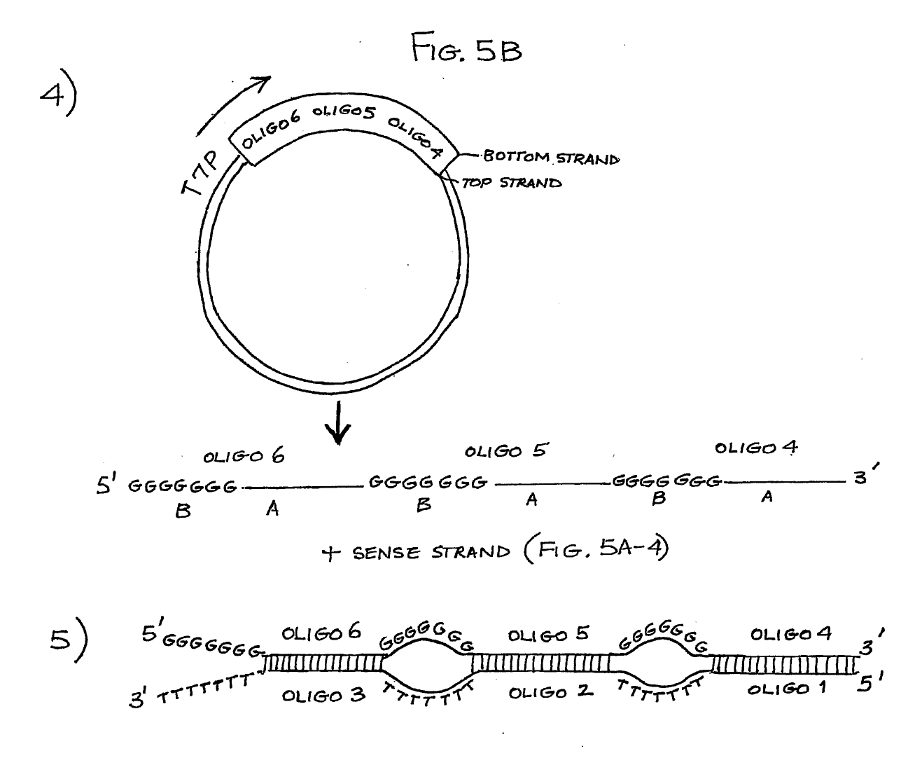

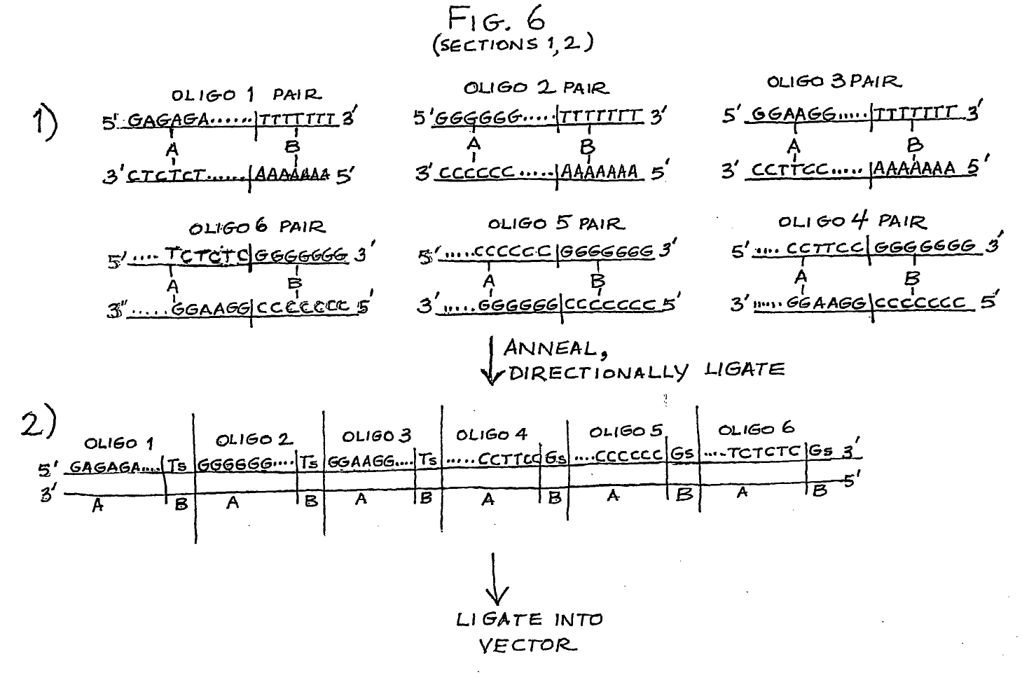

The aforementioned applications disclose a schematic illustration of the RNA stress response pathway, also known as the Type 1 interferon response (see, e.g., FIG. 2 of U.S. Ser. No. 60/375,636; and FIG. 4 of U.S. Ser. No. 10/062,707; U.S. Published Application 2002/0132257; and EP1229134). The pathway is branched and RNA mediated induction/activation can occur at multiple points in the pathway. RNA (dsRNA and other structures) can act to elicit the production of alpha and/or beta interferon in most cell types. Early and key events in the interferon response pathway include interferon-mediated activation of the Jak-Stat pathway, which involves tyrosine-phosphorylation of STAT proteins (STATs). Activated STATs translocate to the nucleus and bind to specific sites in the promoters of IFN-inducible genes thereby effecting transcription of these genes, the expression of which acts in concert to push the cell towards apoptosis or to an anti-proliferative state. There are hundreds of interferon-stimulated genes but only two of the better characterized ones, PKR and 2′5′-OAS, have been shown. RNA can also activate the pathway in an interferon- and STAT-independent manner. In addition, dsRNA/structured RNA can also activate inactive PKR and 2′5′-OAS which are constitutively expressed in many cell types.

Activation of this undesired RNA stress response may require a specific dsRNA sub-cellular localization, higher order structure, and/or amount of cellular dsRNA. For example, we have developed an in vivo expression system for dsRNA (e.g., long dsRNA over 100 base-pairs, desirably over 200 base-pairs, and more desirably over 600 base-pairs) that efficiently induces PTGS without inducing/activating the RNA stress response pathway. Using this system, we have demonstrated the long-term suppression of prostate specific antigen (PSA) and secreted human placental alkaline phosphatase in a human cell line.

The present invention features a variety of novel methods and nucleic acids for silencing genes that produce few, if any, toxic side-effects. In particular, these methods involve administering to a cell or animal an agent that provides one or more double-stranded RNA (dsRNA) molecules that have substantial sequence identity to a region of a target nucleic acid sequence and that specifically inhibit the expression of the target gene. In some embodiments, a portion or all of the dsRNA molecules are located in the cytoplasm and thus mediate post-transcriptional gene silencing (PTGS). In certain embodiments, a portion or all of the dsRNA molecules are located in the nucleus and mediate transcriptional gene silencing (TGS). For TGS applications, the dsRNA desirably includes a regulatory sequence (e.g., a transcription factor binding site or a promoter) and/or a coding sequence, and for PTGS applications, the dsRNA desirably includes a regulatory sequence (e.g., a 5′ or 3′ untranslated region (UTR) of an mRNA) and/or a coding sequence. For methods in which the dsRNA is made in the nucleus and PTGS is desirable, the dsRNA may optionally include one or more constitutive transport element (CTE) sequences or introns to promote transport of the dsRNA into the cytoplasm and/or include a polyA tail to promote dsRNA stability. Desirably, the same dsRNA mediates both TGS and PTGS. In other embodiments, one or more dsRNA molecules that mediate TGS and one or more dsRNA molecules that mediate PTGS are used.

A variety of methods have been developed to inhibit or prevent an interferon or RNA stress response. One such method is based on the surprising discovery that short dsRNA molecules (e.g., dsRNA molecules containing a region of between 11 and 40 nucleotides in length that is in a double-stranded conformation) can be used to inhibit the PKR/interferon/stress/cytotoxicity response induced by other dsRNA molecules (e.g., short or long dsRNA molecules homologous to one or more target genes) in vertebrate cells, tissues, and organisms (See U.S. Ser. No. 60/375,636; filed Apr. 26, 2002 and U.S. Ser. No. 10/425,006 filed Apr. 28, 2003, “Methods of Silencing Genes Without Inducing Toxicity”, C. Pachuk, both of which are incorporated herein by reference). In particular, two short dsRNA molecules prevented the toxic effects that are normally induced by the dsRNA poly (I)(C). Thus, these methods inhibit the induction of non-specific cytotoxicity and cell death by dsRNA molecules (e.g., exogenously introduced long dsRNA molecules) that would otherwise preclude their use for gene silencing in vertebrate cells and vertebrates.

Other approaches for dsRNA-mediated gene silencing without induction of the interferon response involve intracellular expression, either in the cytoplasm or the nucleus, of dsRNA (e.g., a long dsRNA) with substantial identity to a target gene. Surprisingly, this method allows for the sustained expression of long dsRNA within cells without invoking the components of the dsRNA stress or type I interferon response pathway. In particular, gene silencing was observed using nuclear expression of dsRNA from RNA polII, RNA polIII, and T7 constructs, and using cytoplasmic expression of dsRNA. Thus, generation of dsRNA in vivo is an efficient and practicable method for inducing long-term gene silencing in mammalian and other vertebrate systems. Furthermore, intracellular expression of long dsRNA was a very potent inducer of gene silencing. For example, long dsRNA was able to down-regulate the expression of target genes by 95% for at least one month. Additionally, long dsRNA may be more effective for some applications than short dsRNA in the degree and/or the duration of gene silencing. Long-term maintenance of the silencing response is important in many silencing applications such as functional genomics and target validation because many cell models for studying gene function and validating gene targets require sustained loss of targeted gene function. Long-term gene silencing is also desirable for many therapeutic purposes.

If desired, expression of a target gene can be further inhibited by RNA replication of dsRNA with substantial identity to the target gene. For example, an RNA dependent-RNA polymerase can be expressed in a cell or animal into which the dsRNA or a vector encoding the dsRNA is introduced. The RNA dependent-RNA polymerase amplifies the dsRNA and desirably increases the number of dsRNA molecules in the cell or animal by at least 2, 5, or 10-fold. The RNA dependent-RNA polymerase is naturally expressed by the cell or animal, is encoded by the same vector that encodes the dsRNA, or is encoded by a different vector. Exemplary RNA dependent-RNA polymerases include viral, plant, invertebrate, or vertebrate (e.g., mammalian or human) RNA dependent-RNA polymerases. In other approaches, long-term gene silencing is enhanced by expressing the dsRNA from a vector that has an origin of replication that permits replication of the vector in the cell or animal. Desirably, the vector is maintained in the progeny of the cell or animal after 10, 30, 50, 100, or more cell divisions or after one week, one month, six months, or one year.

Additionally, gene silencing can be enhanced by using dsRNA molecules with single-stranded mismatched regions to silence a target gene. In order to facilitate the generation of short dsRNA molecules which are active in gene silencing via RNA interference or other gene silencing pathways, the invention provides novel methods for generation of constructs encoding RNA duplexes or hairpins with mismatches. The sites of mismatches in the RNA are cleavage sites for the single-stranded RNA-specific RNAses. Alternatively, dsRNA can be generated as hairpin RNA from an “udder-structured” RNA which contains multiple short hairpin-loop structures situated in tandem but separated by short spacer sequences susceptible to cleavage by single-strand specific RNAses.

Duplex RNA or hairpin RNA molecules desirably have double-stranded stretches punctuated by regions that are not double-stranded. The double-stranded regions are from desirably from 19 to 100, 19 to 75, 19 to 50, 19 bp to 30 bp, or 19 bp to 25 bp in length. The mismatched regions are desirably from 1 nt to 100 nucleotides, a desirable embodiment being 1-50 nucleotides and the most desirable embodiment being 1-10 nucleotides. The length of the original RNA is from about 40 nucleotides to 10,000 nucleotides.

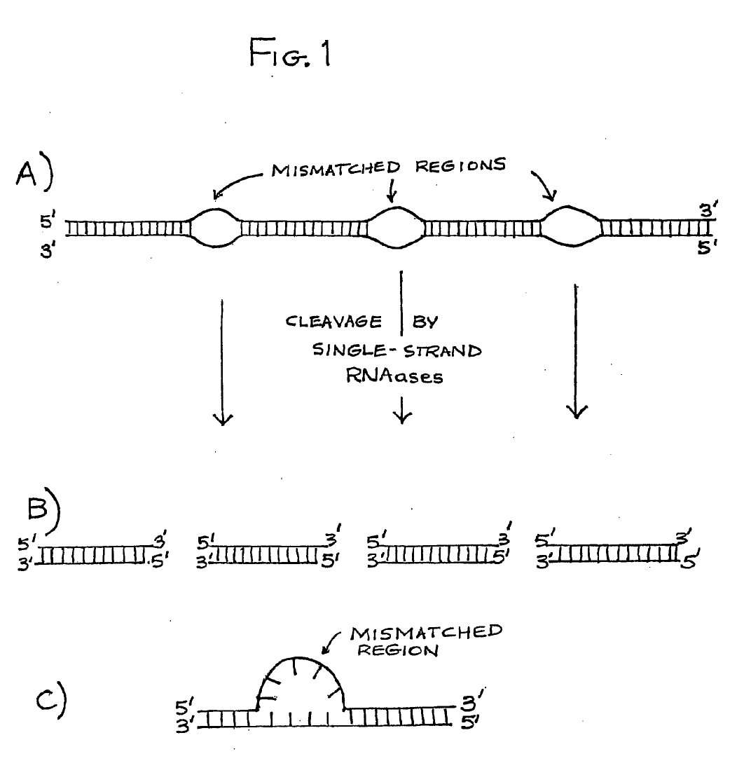

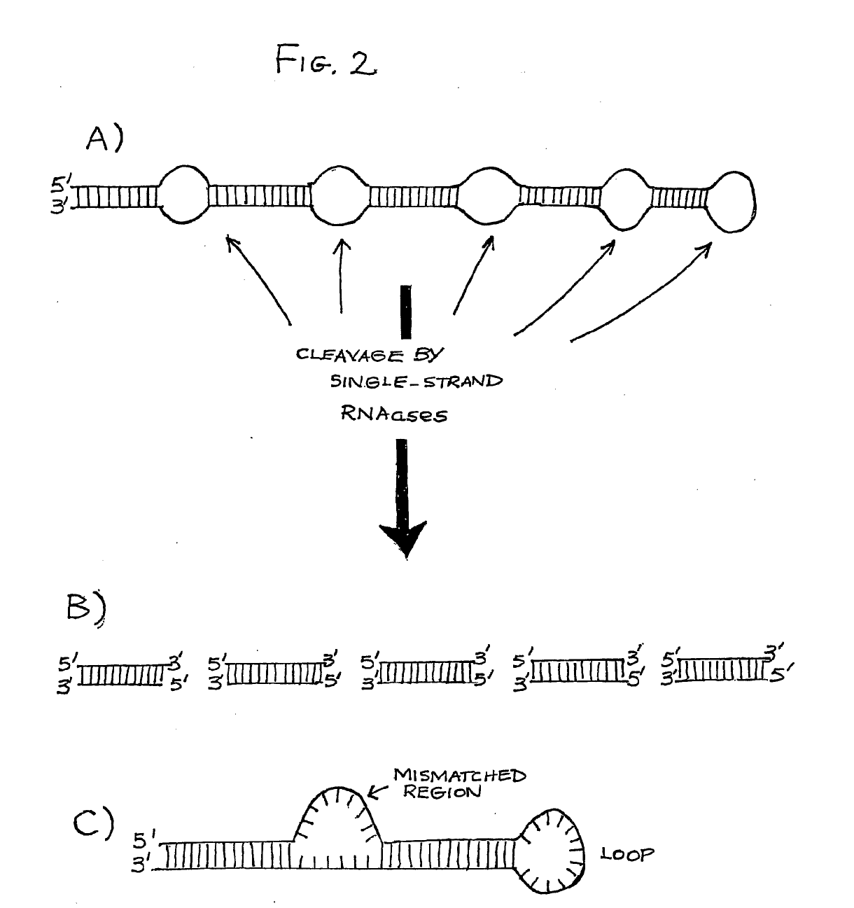

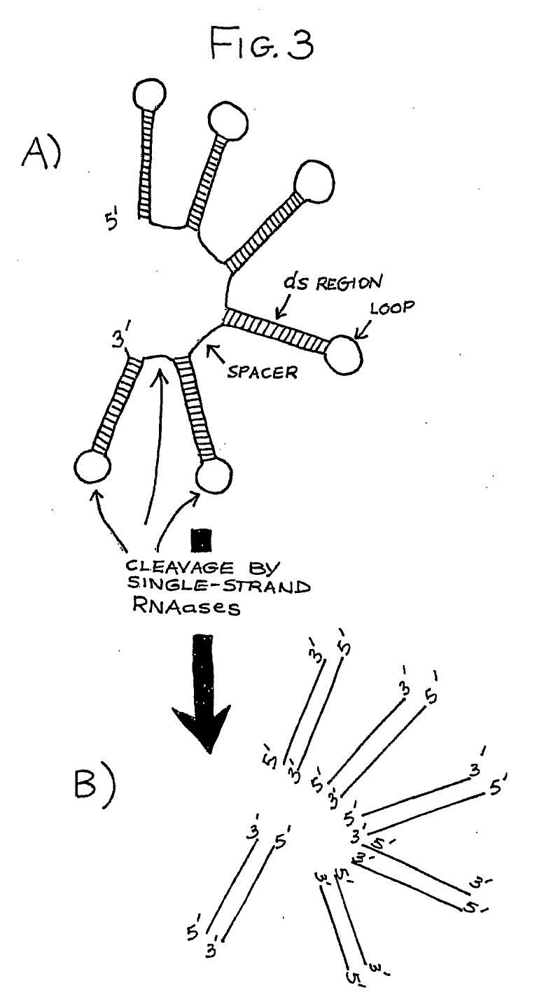

Such molecules are cleaved in the mismatched regions by cellular single-strand specific RNAses to yield double-stranded duplexes (see, e.g., FIGS. 1-3). An array of smaller dsRNA duplexes can therefore be generated from a larger significantly double-stranded duplex or hairpin dsRNA. The smaller dsRNA duplexes can be blunt ended or contain 5′ and or 3′ overhangs. The minimal desirable size of the duplex is 19 base-pairs. Such an invention is useful for situations in which the dsRNA nuclease, Dicer, or its homologues are not present in sufficient amounts, or are not of sufficient activity to process larger dsRNA molecules into smaller dsRNA duplexes. It is these smaller duplexes that are part of the RISC complex which is required for RNAi, also known as PTGS. This invention therefore enables the use of long dsRNA for RNAi purposes under conditions in which Dicer is either not available in sufficient quantities or is not of sufficient activity to process large dsRNA into the smaller dsRNA duplexes. These dsRNA molecules can be used for either PTGS or TGS.

The DNA sequences encoding such RNA molecules are cloned into vectors such that the RNA is transcribed from one or more promoters. Exemplary promoters and vector systems include the T7 RNA polymerase promoter, RNA Pol 1, RNA pol II, RNA pol III promoters, and viral promoters. The duplex RNAs can be generated by using separate cistrons to express the sense and antisense RNA. The cistrons can be located on separate plasmids or on the same plasmid (see, e.g., FIGS. 4A and 4B). The hairpin RNAs are transcribed from one promoter.

Another method for enhancing the generation of short dsRNA molecules which are active in gene silencing vis a vis RNA interference or other gene silencing pathway involves expressing dicer or a dicer homologue in cells with a dsRNA molecule (e.g., long dsRNA) that has substantial sequence identity to one or more target genes. The advantage of co-expressing dicer is that in situations in which endogenous dicer or a dicer homologue is not expressed in adequate levels to process dsRNA (e.g., long dsRNA) into siRNAs, co-expression from a dicer expression vector can supplement these levels, enabling more efficient processing of dsRNA (e.g., long dsRNA) into siRNA. In one embodiment, mouse dicer is co-expressed in vitro in any mammalian cell line or in vivo in mice or in humans. In another embodiment, human dicer is expressed in vitro in a human cell line or in vivo in a human. These methods can be used to express exogenous dicer in a cell, tissue, or animal (e.g., a mammal, such as a human) or to over-express endogenous dicer under the control of a heterologous promoter in a cell, tissue, or animal (e.g., a mammal, such as a human). The cloning of murine and human dicer is described in further detail in Example 16.

Additionally, gene silencing can be enhanced by using other partial or full RNA hairpins to silence a target gene. In some circumstances dsRNA may be generated more efficiently from a single-stranded RNA with inverted repeat sequences that promote formation of a dsRNA hairpin structure from two separate RNA molecules that must hybridize in vitro or in vivo to form dsRNA. In various embodiments, the dsRNA is a partial RNA hairpin that has a single-stranded overhang or a full RNA hairpin without a single-stranded overhang. In the hairpins, one region of the dsRNA molecule has substantial sequence identity to all or a portion of a target nucleic acid sequence (e.g., all or a portion of a gene, a gene promoter, or all or a portion of a gene and its promoter) and is base-paired to another region of interest that has substantial complementarity to the target nucleic acid sequence. If desired, the dsRNA can include additional base-paired regions to increase the efficiency of hairpin formation; for example, the dsRNA can include a loop that is flanked by a base-paired helix which promotes hairpin formation.

The invention also provides novel methods for generating hairpins in vitro or in vivo. These methods involve producing a partial hairpin that has a single-stranded overhang and extending the partial hairpin so that the single-stranded overhang decreases in size. In particular, the partial hairpin has a 3′ end that is base-paired with another region in the partial hairpin, and the 3′ end of the partial hairpin is extended by an RNA dependent-RNA polymerase (e.g., a viral, plant, invertebrate, or vertebrate such as mammalian or human RNA dependent-RNA polymerase). See the teaching of U.S. Provisional Application, 60/399,998, filed 31 Jul. 2002, PCT/US03 . . . , filed Jul. 31, 2003, “Double-stranded RNA Constructs and Structures and Methods for Generating and Using the Same.”, incorporated herein by reference.

The above dsRNA molecules and vectors can be used in a variety of methods for treating, stabilizing, or preventing a disease or disorder in an animal (e.g., an invertebrate or a vertebrate, such as a mammal, e.g., a human). In these methods, a dsRNA or a vector encoding a dsRNA that has substantial sequence identity to all or a region of a target nucleic acid associated with the disease or disorder, and that specifically inhibits the expression of the target gene, is administered to the animal. In some embodiments, the target gene is a gene associated with cancer, such as an oncogene, or a gene encoding a protein associated with a disease, such as a mutant protein, a dominant negative protein, or an overexpressed protein. Moreover, the dsRNA molecules can be used to treat, stabilize, or prevent an infection by a pathogen such as a virus, a bacterium, a yeast, or a fungus. In some embodiments, the target nucleic acid is a gene of the pathogen that is necessary for replication and/or pathogenesis, or a gene encoding a cellular receptor necessary for a cell to be infected by the pathogen.

The invention also features the use of the above dsRNA molecules and dsRNA expression vectors in methods which utilize dsRNA-mediated gene silencing for functional genomics applications, including high throughput methods of using dsRNA-mediated gene silencing to identify a nucleic acid molecule that modulates a detectable phenotype of a cell, e.g., a function of the cell, expression of a target gene, or biological activity of a target polypeptide. These methods involve transfection of libraries of dsRNA molecules or libraries of vectors encoding dsRNA molecules into cells to inhibit gene expression. The inhibition of gene expression modulates a detectable phenotype of a cell and allows the nucleic acid molecule responsible for the modulation to be readily identified.

### EXAMPLES

The following examples are to illustrate the invention. They are not meant to limit the invention in any way. For example, it is noted that any of the following examples can be used with dsRNA molecules of any length and structure, including any of the dsRNA structures of the invention, which include one or more double-stranded regions (preferably two or more double-stranded regions), one strand of which has substantial sequence identity to all or a region of a target nucleic acid sequence (e.g., all or a portion of a gene, a gene promoter, or all or a portion of a gene and its promoter), and which includes at least one mismatched region. The methods of the present invention can be readily adapted by one skilled in the art to utilize multiple dsRNA molecules and/or multiple dsRNA expression constructs to inhibit multiple target nucleic acid molecules (e.g., one or more target genes). Any of the dsRNA molecules, target nucleic acid molecules, or methods described in, e.g., in U.S. Published Application 2002/0132257 and European Published Application EP1229134, “Use of post-transcriptional gene silencing for identifying nucleic acid sequences that modulate the function of a cell”, the teaching of which is hereby incorporated by reference, can also be used in the present methods.

While the use of the present invention is not limited to vertebrate or mammalian cells, such cells can be used to carry out the methods described herein. Desirably, the vertebrate (e.g., mammalian) cells used to carry out the present invention are cells that have been cultured for only a small number of passages (e.g., less than 30 passages of a cell line that has been obtained directly from American Type Culture Collection), or are primary cells. In addition, vertebrate (e.g., mammalian) cells can be used to carry out the present invention when the dsRNA being transfected into the cell is not complexed with cationic lipids.

### Example 1

### Transcriptional and Post-Transcriptional Gene Silencing

Transcriptional gene silencing (TGS) is a phenomenon in which silencing of gene expression occurs at the level of RNA transcription. Double-stranded RNA mediates TGS as well as post-transcriptional gene silencing (PTGS), but the dsRNA needs to be located in the nucleus, and desirably is made in the nucleus in order to mediate TGS. PTGS occurs in the cytoplasm. A number of dsRNA structures and dsRNA expression vectors have been delineated herein that can mediate TGS, PTGS, or both. Various strategies for mediating TGS, PTGS, or both are summarized below.

All of the cytoplasmic dsRNA expression vectors described herein mediate PTGS because they generate dsRNA in the cytoplasm where the dsRNA can interact with target mRNA. Because some of the dsRNA made by these vectors translocate to the nucleus via a passive process (e.g., due to nuclear envelope degeneration and reformation during mitosis), these vectors are also expected to affect TGS at a low efficiency in dividing cells. RNA PolII vectors express RNA molecules in the nucleus with various abilities to enter the cytoplasm.

If desired, one or more constitutive transport element (CTE) sequences can be added to enable cytoplasmic transport of the different effector RNA molecules (e.g., hairpins or duplexes) that are made in the nucleus by RNA PolII. A CTE can be used instead of and/or in addition to an intron and/or polyA sequence to facilitate transport. A desirable location for the CTE is near the 3′ end of the RNA molecules. If desired, multiple CTE sequences (e.g., 2, 3, 4, 5, 6, or more sequences can be used). A preferred CTE is from the Mason-Pfizer Monkey Virus (U.S. Pat. Nos. 5,880,276 and 5,585,263).

Vectors encoding a functional intron or CTE in combination with a polyadenylation signal more efficiently export dsRNA to the cytoplasm. Vectors with (i) only an intron or CTE and no polyadenylation signal, or (ii) with only a polyadenylation signal and no intron or CTE, export RNA to the cytoplasm with a lesser efficiency, resulting in less RNA in the cytoplasm and a lower efficiency for PTGS. Vectors encoding RNA without an intron, CTE, and polyadenylation signal result in RNA molecules that are the least efficiently transported to the cytoplasm. The lower the level of cytoplasmic transport of RNA, the more RNA retention in the nucleus and the higher efficiency with which TGS is induced. Therefore, all of these vectors induce PTGS and TGS with varying efficiencies according to the level of cytoplasmic transport and nuclear retention, respectively, as described above.

RNA PolIII vectors, which can have one or more introns or no introns and can have a polyA tail or no polyA tail, encode RNA molecules that are made in the nucleus and are primarily retained in the nucleus. This nuclear RNA induces TGS. However, a percentage of the transcribed RNA reaches the cytoplasm and can therefore induce PTGS. For TGS induction, the dsRNA desirably contains a promoter, or a subset of a promoter sequence, and is retained in the nucleus. Alternatively, the dsRNA may contain only coding or UTR sequence, or may desirably contain a combination of coding or UTR sequence and promoter sequence. Such “fusion target” dsRNAs may contain, e.g., both a promoter sequence and a linked gene sequence to be targeted for concurrent TGS and PTGS. For PTGS, the dsRNA contains sequence derived from an RNA (e.g., coding or UTR sequence from an mRNA) and does not have to contain promoter sequence. In addition, more efficient PTGS is induced by vectors that enable cytoplasmic transcription or by vectors that result in more efficiently cytoplasmically transported RNA. If desired, PTGS and TGS can be induced simultaneously with a combination of these vectors using the methods described herein and techniques known to those skilled in the art.

Any of the vectors described herein, in “Multiple-Compartment Eukaryotic Expression Systems”, C. Pachuk and C. Satishchandran, U.S. Provisional Application Ser. No. 60/497,304, filed Aug. 22, 2003, incorporated herein by reference, or any other standard vector can also be used to generate the dsRNA structures of the invention, and used in the present methods.

### Example 2

### Exemplary Methods for Enhancing Post-Transcriptional Gene Silencing

To enhance PTGS by dsRNA transcribed in the nucleus by RNA PolII, one or more introns and/or a polyadenylation signal can be added to the dsRNA to enable processing of the transcribed RNA. This processing is desirable because both splicing and polyadenylation facilitate export from the nucleus to the cytoplasm. In addition, polyadenylation stabilizes RNA PolII transcripts. In some embodiments, a prokaryotic antibiotic resistance gene, e.g., a zeomycin expression cassette is located in the intron. Other exemplary prokaryotic selectable markers include other antibiotic resistance genes such as kanamycin, including the chimeric kanamycin resistance gene of U.S. Pat. No. 5,851,804, aminoglycosides, tetracycline, and ampicillin. The zeomycin gene is under the regulatory control of a prokaryotic promoter, and translation of zeomycin in the host bacterium is ensured by the presence of Shine-Dalgarno sequences located within about 10 base-pairs upstream of the initiating ATG. Alternatively, the zeomycin expression cassette can be placed in any location between the inverted repeat sequences of the hairpin (i.e., between the sense and antisense sequences with substantial identity to the target nucleic acid to be silenced).

Although inverted repeat sequences are usually deleted from DNA by DNA recombination when a vector is propagated in bacteria, a small percentage of bacteria may have mutations in the recombination pathway that allow the bacteria to stably maintain DNA bearing inverted repeats. In order to screen for these infrequent bacteria, a zeomycin selection is added to the culture. The undesired bacteria that are capable of eliminating inverted repeats are killed because the zeomycin expression cassette is also deleted during recombination. Only the desired bacteria with an intact zeomycin expression cassette survive the selection.

After the DNA is isolated from the selected bacteria and inserted into eukaryotes (e.g., mammalian cell culture) or into animals (e.g., adult mammals) for expression of RNA, the intron is spliced from the RNA transcripts. If the zeomycin expression cassette is located in the intron, this cassette is removed by RNA splicing. In the event of inefficient splicing, the zeomycin expression cassette is not expressed because there are no eukaryotic signals for transcription and translation of this gene. The elimination of the antibiotic resistance cassette is desirable for applications involving short dsRNA molecules because the removal of the cassette decreases the size of the dsRNA molecules. The zeomycin cassette can also be located beside either end of an intron instead of within the intron. In this case, the zeomycin expression cassette remains after the intron is spliced and can be used to participate in the loop structure of the hairpin. These RNA PolI transcripts are made in the nucleus and transported to the cytoplasm where they can effect PTGS. However, some RNA molecules may be retained in the nucleus. These nuclear RNA molecules may effect TGS. For TGS applications, the encoded dsRNA desirably contains a promoter or a subset of a promoter. In order to more efficiently retain RNA within the nucleus, the intron and/or polyadenylation signal can be removed.

Another strategy for both cytoplasmic and nuclear localization is to use “upstream” or internal RNA PolIII promoters (see, e.g., Gene regulation: A Eukaryotic Perspective, 3^{rd} ed., David Latchman (Ed.) Stanley Thornes: Cheltenham, UK, 1998). These promoters result in nuclear transcribed RNA transcripts, some of which are exported and some of which are retained in the nucleus and hence can be used for PTGS and/or TGS. These promoters can be used to generate hairpins, including the partial and forced hairpin structures of the invention, or duplex RNA through the use of converging promoters or through the use of a two vector or two cistronic system. One promoter directs synthesis of the sense strand, and the other promoter directs synthesis of the antisense RNA. The length of RNA transcribed by these promoters is generally limited to several hundred nucleotides (e.g., 250-500). In addition, transcriptional termination signals may be used in these vectors to enable efficient transcription termination.

Exemplary Vector Encoding a dsRNA with an Intron Containing an Antibiotic Resistance Gene

The human cytomegalovirus major immediate-early protein intron I (Accession No. M21295) was PCR amplified using the following forward primer KpnI-intron-f (5′-CGC GGG TAC CAA CGG TGC ATT GGA ACG C-3′) and the reverse primer NheI-intron-r (5′-ATC GGC TAG CGG ACG GTG ACT GCA GAA AAG ACC CAT GG-3′). These primers amplify the region from nucleotides 594 to 1469 and introduce a KpnI site on the 5′ end and a NheI site on the 3′ end of the intron. This product was inserted into the EcoRV site of pBSII KS(+) (Stratagene, LaJolla, Calif.) to create the vector pBS-IVS.

The Zeocin gene is commercially available (Invitrogen, pcDNA3.1 (+)Zeo). The gene with a prokaryotic promoter was PCR amplified using the forward primer 5′ ZeoSphI (5′-ATG CAT GCC GTG TTG ACA ATT AAT CAT CGG C-3′) and the reverse primer 3′ ZeoHpaI (5′-ATG TTA ACC ACG TGT CAG TCC TGC TCC TCG-3′) using pCDNA (+Zeo) (Invitrogen). This PCR product was cleaved with SphI and HpaI, and the fragment was inserted into the hCMV intron A (Genbank accession number M21295, nucleotides 594-1470) contained at the SphI and HpaI sites to create the vector pBS-Iz. This insertion incorporates Zeocin into the intron A sequence in the same orientation and leaves the intron A acceptor and donor sites and their flanking regions intact (IVS-Zeocin).

The IVS-Zeocin (Iz) was excised from pBS-Iz using the enzymes KpnI and NheI and the isolated fragment was inserted into an expression vector downstream of a human cytomegalovirus promoter (Genbank accession number AF105229). Downstream of the insertion site, the vector contained the bovine growth hormone polyadenylation signal. The Iz was inserted into the KpnI and NheI sites of the vector MCS; this construct maintains the native orientation of Iz with respect to the promoter to allow for processing of the RNA and excision of the intronic sequence. The encoded RNA is also predicted to be polyadenylated. This vector was named pCMV-Iz.

Secreted Alkaline Phosphatase (SEAP; Genbank accession number U89938) was PCR amplified using the forward primer KpnI-SEAP-f (5′-AGC CGG TAC CCT ATT CCA GAA GTA GTG AGG-3′) and the reverse primer SEAP5′Xho (5′-CGT AAC TCG AGC ACT GCA TTC TAG TTG TGG-3′). This PCR reaction amplifies the full length SEAP and introduces a KpnI site into the 5′ end and a XhoI site into the 3′ end. The product was sub-cloned into pBSII KS(+) that was cleaved with EcoRI to create the vector pBS-SEAPKX. Full length SEAP was excised from pBS-SEAPKX using KpnI and XhoI and inserted into pCMV-Iz. This insertion was in the reverse orientation and was upstream of the Iz sequence using the KpnI and XhoI sites of the pCMV-Iz vector. A SEAPΔ PCR product was generated using the forward primer NheI-SEAP-f (5′-AGC CGC TAG CCT ATT CCA GAA GTA GTG AGG-3′) and SEAP3′XhoI. This reaction produces a 650 base-pair fragment of SEAP with an NheI site on the 5′ end and an XhoI site on the 3′ end. The SEAP NheI/XhoI PCR product was cut with NheI and XhoI and inserted into pCMV-SEAP-Iz at the NheI and SalI restriction sites. This insertion was in the forward orientation and was downstream of the Iz sequence, generating the vector pCMV-SEAP-Iz-SEAPΔ. Selection on media containing 35 ug/ml Zeocin resulted in the successful replication of a vector containing a 650-700 base-pair inverted repeat. The replication of this desired vector occurred in DH5α cells under the conditions tested.

This method has also been performed with mIL-12p40 (full length and 500 base-pair segments) and mCK-M. Additionally, this method was performed in two different vector systems utilizing both the T7 and the hCMV promoter system. Theoretically, this method can be performed for any vector, any promoter, any polyA signal, and any drug resistance gene or any positive selection marker inserted within or near any intron sequence that contains a functional acceptor and donor site.

### Example 3

### Exemplary Methods for the Generation of dsRNA In Vivo

Exemplary intracellular expression systems for sustained expression of dsRNA include cytoplasmic expression systems, e.g., a T7 promoter/T7 RNA polymerase, mitochondrial promoter/mitochondrial RNA polymerase, or RNA polII expression system. Other possible cytoplasmic expression systems use exogenously introduced viral or bacteriophage RNA polymerases and their cognate promoters or endogenous polymerases such as the mitochondrial RNA polymerase with their cognate promoters. In another embodiment, the sustained long dsRNA intracellular expression system is a nuclear expression system, such as an RNA polI, RNA polII, or RNA polIII expression system.

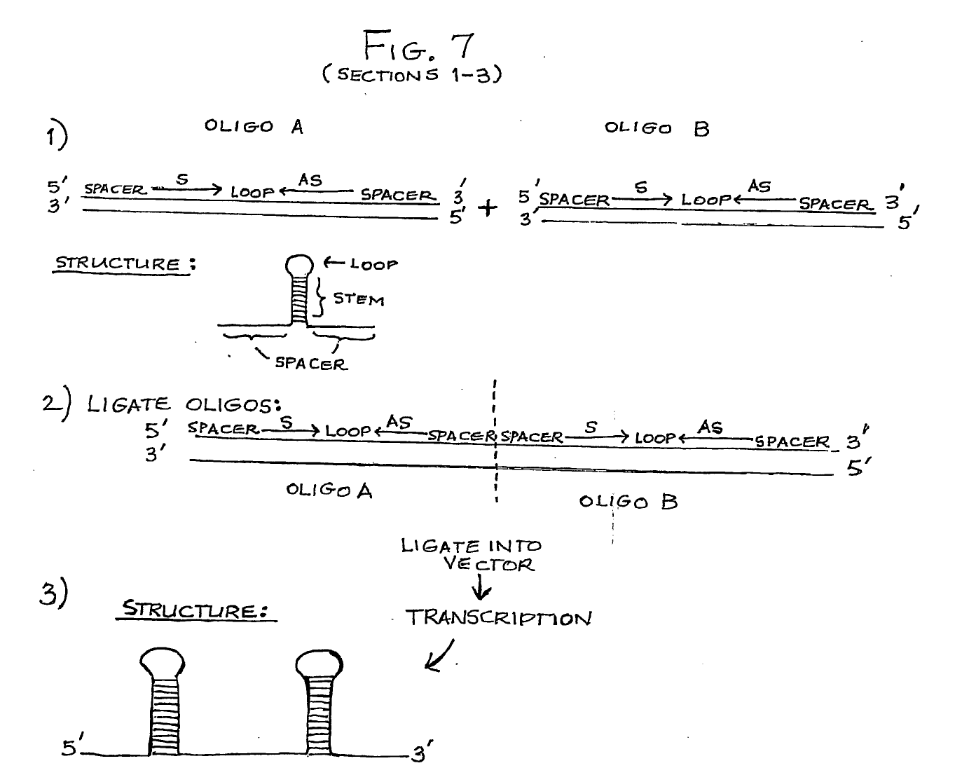

Expression in eukaryotic cells is complicated by the existence of subcellular compartments, including functional compartments. This results in a situation where populations of expression constructs (frequently, the majority of the expression constructs which make it into the cell) are non-functional simply because they are located in subcellular compartments in which the encoded promoters are not active. For example, promoters, including the widely used HCMV promoter, which are driven by RNA polymerase II (RNA pol II) are active only in the nucleus but not in the cytoplasm where the greatest number of the expression constructs are located. The majority of such expression constructs in the cell (those in the cytoplasmic compartments) are therefore not active. By including two or more, e.g., several, promoters each active in a different subcellular compartment of a eukaryote, it is possible to engineer a multi-compartment eukaryotic expression system, e.g. a plasmid or combination of plasmids, that are transcriptionally active no matter where in the cell the plasmid(s) is localized. In some aspects, a single expression construct can be designed to be transcriptionally active in e.g., two, three, four, or even all subcellular compartments of a eukaryotic cell in which transcription occurs, or can be made to occur. In other aspect of the invention, a eukaryotic expression system comprising two or more expression constructs can be designed to include a combination of different-subcompartment promoters to be transcriptionally active in e.g., two, three, four, or even all subcellular compartments, including functional domains, within a single subcellular compartment, of a eukaryotic cell in which transcription occurs, or can be made to occur. Desirable expression constructs to express the dsRNA molecules of the invention having double-stranded regions interspersed by mismatched regions may be designed to be active in two or more compartments of a cell. For example, a plasmid expression vector may be constructed which contains a sequence as described in FIG. 6 placed under the control of two or more promoters. At least two promoters are used, each active in a different physical subcellular compartment and/or a separate functional domain of a subcellular compartment, so that there is a higher likelihood of the sequence being transcribed regardless of the subcellular environment to which the vector localizes following transfection in vitro or in vivo. For example, such a plasmid may include one copy of a sequence encoding a hairpin dsRNA, operably linked to a T7 promoter, and a second copy of the same sequence under the control of an RNA pol III promoter, such as the human U6 promoter. Each transcription unit includes the appropriate terminator sequence, T7t and U6t, respectively. The promoters may be divergent with respect to each other (i.e., transcription proceeds in the same direction) or the T7 promoter and the U6 promoter may flank the encoded hairpin dsRNA sequence and be convergent with respect to each other. See further the teaching of “Multiple-Compartment Eukaryotic Expression Systems”, C. Pachuk and C. Satishchandran, U.S. Provisional Application Ser. No. 60/497,304, filed Aug. 22, 2003, incorporated herein by reference.

Constructs for Intracellular Expression of dsRNA in Vertebrate Cells

A variety of expression constructs capable of expressing dsRNA intracellularly in a vertebrate cell can be utilized to express the various at least partially double-stranded RNA molecules, including the dsRNAs with mismatched regions described in this application, including those which are forced and partial hairpin structures of the invention (as described in more detail in U.S. Provisional Application 60/399,998 filed 31 Jul. 2002, and PCT/US2003 . . . , filed 31 Jul. 2003), and long dsRNA molecules having a double-stranded region desirably at least 50 base-pairs, more desirably greater than 100 base-pairs, still more desirably greater than 200 base-pairs, including sequences of 1, 2, 3, 4, 5, or more kilobases that are within the maximum capacity for a particular plasmid, e.g., 20 kilobases, or as appropriate for a viral or other vector.

Expression vectors designed to produce dsRNA can be a DNA single-stranded or double-stranded plasmid or vector. Expression vectors designed to produce dsRNA as described herein may contain sequences under the control of any RNA polymerase, such as a mitochondrial RNA polymerase, RNA polI, RNA polIII, or exogenously introduced viral or bacteriophage RNA polymerase. Vectors may be desirably designed to utilize an endogenous mitochondrial polymerase (e.g., human mitochondrial RNA polymerase together with the corresponding human mitochondrial promoter). Mitochondrial polymerases may be used to generate capped dsRNA through expression of a capping enzyme or generate uncapped dsRNA transcripts in vivo. RNA polI, RNA polII, and RNA polIII transcripts may also be generated in vivo. Such RNA molecules may be capped or not, and if desired, cytoplasmic capping may be accomplished by various means including use of a capping enzyme such as a vaccinia capping enzyme or an alphavirus capping enzyme. DNA expression vectors are designed to contain one promoter or multiple promoters in combination (mitochondrial, RNA polI, RNA polII, RNA polIII, viral, bacterial or bacteriophage promoters) along with their cognate RNA polymerases (e.g., T3, T7, or SP6 bacteriophage systems). Desirably, RNA polII systems use a segment encoding a dsRNA that has an open reading frame greater than about 300 nucleotides to avoid degradation in the nucleus. Further information concerning constructs for the intracellular production of the RNA molecules of the invention, including viruses and viral sequences that may be manipulated to provide the required RNA molecule to the mammalian cell in vivo (e.g., alphavirus, adenovirus, adeno-associated virus, baculovirus, delta virus, pox viruses, hepatitis viruses, herpes viruses, papova viruses such as SV40, poliovirus, pseudorabies virus, retroviruses, vaccinia viruses, positive and negative stranded RNA viruses, viroids, and virusoids) can be found in, for example, WO 00/63364, which is incorporated herein by reference.

Any other DNA-dependent RNA polymerase (e.g., a viral, plant, invertebrate, or vertebrate polymerase) can be used (see, e.g., Table 2). In some embodiments, the dsRNA transcribed by the polymerase is expressed under the control of a promoter from the same organism, species, or genus from which the polymerase coding sequence was obtained.

TABLE 2
DNA dependent RNA polymerases

| | Genbank |
|---|---|
| Source of DNA-dependent RNA Polymerase | Accession No. |
| African swine fever virus NP1450L gene encoding | Z21489 |
| RNA polymerase largest subunit | |
| African swine fever virus, complete genome | NC_01659 |
| African swine fever virus, complete genome | U18466 |
| African swine fever virus EP1242L gene encoding | Z21490 |
| RNA polymerase second largest subunit | |
| Rabbit fibroma virus, complete genome | NC_001266 |
| Vaccinia virus, complete genome | NC_001559 |
| Autographa californica nucleopolyhedrovirus, complete | NC_001623 |
| genome | |
| Mastigamoeba invertens DNA-dependent RNA | AF083338 |
| polymerase II largest subunit (RPB1) gene, partial cds | |
| G. lamblia rpoA3 gene for subunit A of DNA dependent | X6032 |
| RNA polymerase III | |
| E. gracilis chloroplast RNA polymerase rpoB-rpoC1- | X17191 |
| rpoC2 operon | |
| Listeria monocytogenes unidentified gene and partial | Y16468 |
| rpoB gene | |
| Maize chloroplast RNA polymerase (rpoC1) gene, 5′ | M31207 |
| end | |
| Maize chloroplast RNA polymerase (rpoC2) gene, 5′ | M31208 |
| end | |
| Maize chloroplast RNA polymerase (rpoB) gene, 5′ end | M31206 |

Exemplary promoter and coding sequences of target nucleic acids are listed in Table 3 (see below). Other promoters and coding sequences can be readily identified by one skilled in the art from published databases or references or from standard methods such as standard sequence analysis techniques. For targeting a promoter, a dsRNA of, e.g., at least 19-30 nucleotides in length can be designed to include the TATA box or CAT box within the dsRNA (see, e.g., Molecular Cell Biology, Lodish (ed.) 3rd edition, Scientific American books: New York, 1995). In other embodiments, a region of, e.g., at least 350, 500, 750, 1000, 1500, 2000, or 2500 nucleotides upstream of the coding sequence can be used to target the promoter and/or other regulatory elements of a nucleic acid sequence of interest. In certain desirable embodiments, both a promoter and a coding sequence will be targeted in the same dsRNA or dsRNA expression construct.

TABLE 3
Exemplary Target Genes and Promoters, and Genomes Containing
Same

| | | Genbank Accession |
|---|---|---|
| Virus | Target Genes and Promoters and Genomes containing same | No. |
| Retroviruses | Human immunodeficiency virus type 2, complete genome | NC_001722.1 |
| | Human immunodeficiency virus type 1, complete genome | NC_001802.1 |
| | Human T-cell lymphotropic virus type 1, complete genome | NC_001436.1 |
| | Human T-cell lymphotropic virus type 2, complete genome | NC_001488.1 |
| Hepatitis B | Hepatitis B virus, complete genome | NC_003977.1 |
| Pox Viruses | Variola virus, complete genome | NC_001611.1 |
| | Vaccinia virus, complete genome | NC_001559.1 |
| Herpesvirus | Human herpesvirus 1, complete genome | NC_001806.1 |
| | Human herpesvirus 2, complete genome | NC_001798.1 |
| Epstein-barr Virus | Epstein-barr virus rna polmerase ii promoter region | J02075.1 |
| | Human herpesvirus 4, complete genome | NC_001345.1 |
| | Epstein-barr virus rna polymerase ii promoter region 12 | J02074.1 |
| | Epstein-barr virus (EBV) genome, strain B95-8 | V01555.1 |
| | Epstein-barr virus rna polymerase ii promoter region 11 | J02073.1 |
| Chicken pox | Human herpesvirus 3, complete genome | NC_001348 |
| Cytomegalovirus | Rat cytomegalovirus, complete genome | NC_002512.2 |
| | Chimpanzee cytomegalovirus, complete genome | NC_003521.1 |
| | Human herpesvirus 6, complete genome | NC_001664.1 |
| | Human herpesvirus 5, genome | NC_001347.1 |
| | Mouse cytomegalovirus 1, complete genome | NC_004065.1 |
| Human | Human papillomavirus type 1a, complete genome | NC_001356.1 |
| Papillomavirus | | |
| | Human papillomavirus type 2a, complete genome | NC_001352.1 |
| | Human papillomavirus type 4, complete genome | NC_001457.1 |
| | Human papillomavirus type 5b, complete genome | NC_001444.1 |
| | Human papillomavirus type 6, complete genome | NC_000904.1 |
| | Human papillomavirus type 8, complete genome | NC_001532.1 |
| | Human papillomavirus type 11, complete genome | NC_001525.1 |
| | Human papillomarvirus type 13, complete genome | NC_001349.1 |
| | Human papillomavirus tpe 16, complete genome | NC_001526.1 |
| | Human papillomavirus type 18, complete genome | NC_001357.1 |
| | Human papillomavirus type 31, complete genome | NC_001527.1 |
| | Human papillomavirus type 33, complte genome | NC_001528.1 |
| | Human papillmavirus type 35, complete genome | NC_001529.1 |
| | Human papillomavirus type 39, complete genome | NC_001535.1 |
| | Human papillomavirus type 41, complete genome | NC_001354.1 |
| | Human papillomavirus type 42, complete genome | NC_001534.1 |
| | Human papillomavirus type 47, complete genome | NC_001530.1 |
| | Human papillomavirus type 51, complete genome | NC_001533.1 |
| | Human papillomavirus type 57, complete genome | NC_001353.1 |
| | Human papillomavirus type 58, complete genome | NC_001443.1 |
| | Human papillomavirus type 63, complete genome | NC_001458.1 |
| | Human papillomavirus type 65, complete genome | NC_001459.1 |
| Adenovirus | Human adenovirus B, complete genome | NC_004001.1 |
| | Ovine adenovirus 7, complete genome | NC_004037.1 |
| | Porcine adenovirus C, complete genome | NC_002702.1 |
| | Bovine adenovirus A, complete genome | NC_002685.1 |
| | Murine adenovirus A, complete genome | NC_000942.1 |
| | Fowl adenovirus D, complete genome | NC_000899.1 |
| | Porcine adenovirus A, complete genome | NC_001997.1 |
| | Bovine adenovirus B, complete genome | NC_001876.1 |
| | Duck adenovirus A, complete genome | NC_001813.1 |
| | Canine adenovirus, complete genome | NC_001734.1 |
| | Human adenovirus A, complete genome | NC_001460.1 |
| | Human adenovirus F, complete genome | NC_001454.1 |
| | Human adenovirus C, complete genome | NC_001405.1 |
| | Fowl adenovirus A, complete genome | NC_001720.1 |
| | Ovine adenovirus A, complete genome | NC_002513.1 |
| | Human adenovirus D, complete genome | NC_002067.1 |
| | Human adenovirus E, complete genome | NC_003266.1 |
| | Frog adenovirus 1, complete genome | NC_002501.1 |
| | Hemorrhagic enteritis virus, complete genome | NC_001958 |
| ParvoVirus | Parvovirus H1, complete genome | NC_001358.1 |
| | Bovine parvovirus, complete genome | NC_001540.1 |
| | Porcine parvovirus strain NADL-2 | NC_001718.1 |
| | Canine parvovirus, complete genome | NC_001539.1 |
| | Goose parvovirus, complete genome | NC_001701.1 |
| | Aleutian mink disease parvovirus, complete genome | NC_001662.1 |
| | Mouse parvovirus 1, complete genome | NC_001630.1 |
| Other viruses | West Nile virus, complete genome | NC_001563.2 |
| | Japanese encephalitis virus (strain JaOArS982), complete | NC_001437.1 |
| | genome | |
| | Dengue virus type 2, complete genome | NC_001474.1 |
| | Dengue virus type 4, complete genome | NC_002640.1 |
| | Dengue virus type 1, complete genome | NC_001477.1 |
| | Dengue virus type 3, complete genome | NC_001475.1 |
| | Yellow fever virus, complete genome | NC_002031.1 |
| | Marburg virus, complete genome | NC_001608.2 |
| | Ebola virus, complete genome | NC_002549.1 |
| | Poliovirus, complete genome | NC_002058.3 |
| | Measles virus, complete genome | NC_001498.1 |
| | Mumps virus, complete genome | NC_002200.1 |
| Picornoviridae | Aichi virus, complete genome | NC_001918.1 |
| | Bovine enterovirus, complete genome | NC_001859.1 |
| | Human enterovirus 70, complete genome | NC_001430.1 |
| | Poliovirus, complete genome | NC_002058.3 |
| | Theiler's encephalomyelitis virus, complete genome | NC_001366.1 |
| | Porcine enterovirus A, complete genome | NC_003987.1 |
| | Foot-and-mouth disease virus SAT 2, genome | NC_003992.1 |
| | Foot-and-mouth disease virus C, complete genome | NC_002554.1 |
| | Equine rhinitis B virus, complete genome | NC_003983.1 |
| | Ljungan virus, complete genome | NC_003976.1 |
| | Human rhinovirus A, complete genome | NC_001617.1 |
| | Human rhinovirus B, complete genome | NC_001490.1 |
| | Hepatitis A virus, complete genome | NC_001489.1 |
| | Equine rhinovirus 3, complete genome | NC_003077.1 |
| | Porcine enterovirus B, complete genome | NC_001827.1 |
| | Human enterovirus A, complete genome | NC_001612.1 |
| | Human enterovirus B, complete genome | NC_001472.1 |
| | Human enterovirus C, complete genome | NC_001428.1 |
| | Human parechovirus 2, complete genome | NC_001897.1 |
| | Foot-and-mouth disease virus O, complete genome | NC_004004.1 |
| | Encephalomyocarditis virus, complete genome | NC_001479.1 |
| | A-2 plaque virus, complete genome | NC_003988.1 |
| | Avian encephalomyelitis virus strain | NC_003990.1 |
| | Mengo virus, complete genome | NC_003989.1 |
| | Human echovirus 1, complete genome | NC_003986.1 |
| | Porcine teschovirus, genome | NC_003985.1 |
| | Equine rhinitis A virus, complete genome | NC_003982.1 |
| Calcivirdae | Norwalk virus, complete genome | NC_001959.1 |
| | Calicivirus strain NB, complete genome | NC_004064.1 |
| | Rabbit hemorrhagic disease virus, complete genome | NC_001481.1 |
| | Feline calicivirus, complete genome | NC_001481.1 |
| | Porcine enteric calicivirus, complete genome | NC_000940.1 |
| | European brown hare syndrome virus, complete genome | NC_002615.1 |
| Astroviridae | Avian nephritis virus, complete genome | NC_003790.1 |
| | Human astrovirus, complete genome | NC_001943.1 |
| | Turkey astrovirus, complete genome | NC_002470.1 |
| | Sheep astrovirus, complete genome | NC_002469.1 |
| Togaviridae | Semliki forest virus, complete genome | NC_003215.1 |
| | Barmah Forest virus, complete genome | NC_001786.1 |
| | Mayaro virus, complete genome | NC_003417.1 |
| | Ross River virus, complete genome | NC_001544.1 |
| | Venezuelan equine encephalitis virus, complete genome | NC_001449.1 |
| | Rubella virus, complete genome | NC_001545.1 |
| | Sindbis virus, complete genome | NC_001547.1 |
| | O'nyong-nyong virus, complete genome | NC_001512.1 |
| | Igbo Ora virus, complete genome | NC_001924.1 |
| | Western equine encephalomyelitis virus, complete genome | NC_003908.1 |
| | Aura virus, complete genome | NC_003900.1 |
| | Salmon pancreas disease virus, complete genome | NC_003930.1 |
| | Eastern equine encephalitis virus, complete genome | NC_003899.1 |
| | Sleeping disease virus, complete genome | NC_003433.1 |
| Flavivirus | Hepatitis C virus, complete genome | NC_001433.1 |
| | Tamana bat virus, genome | NC_003996.1 |
| | West Nile virus, complete genome | NC_001563.1 |
| | Powassan virus, complete genome | NC_003687.1 |
| | Pestivirus Giraffe-1, complete genome | NC_003678.1 |
| | Pestivirus Reindeer-1, complete genome | NC_003677.1 |
| | Apoi virus, genome | NC_003676.1 |
| | Rio Bravo virus, genome | NC_003675.1 |
| | Pestivirus type 2, complete genome | NC_002657.1 |
| | Bovine viral diarrhea virus genotype 2, complete genome | NC_002032.1 |
| | Mosquito cell fusing agent, complete genome | NC_001564.1 |
| | Deer tick virus, genome | NC_003218.1 |
| | Louping ill virus, complete genome | NC_001809.1 |
| | Dengue virus type 2, complete genome | NC_001474.1 |
| | Yellow fever virus, complete genome | NC_002031.1 |
| | Dengue virus type 4, complete genome | NC_002640.1 |
| | Japanese encephalitis virus (strain JaOArS982), complete | NC_001437.1 |
| | genome | |
| | Langat virus, complete genome | NC_003690.1 |
| | Hepatitis GB virus C, complete genome | NC_002348.1 |
| | Dengue virus type 1, complete genome | NC_001477.1 |
| Coronaviridae | Transmissible gastroenteritis virus, complete genome | NC_002306.2 |
| | Murine hepatitis virus, complete genome | NC_001846.1 |
| | Bovine coronavirus, complete genome | NC_003045.1 |
| | Human coronavirus 229E, complete genome | NC_002645.1 |
| | Porcine epidemic diarrhea virus, complete genome | NC_003436.1 |
| | Avian infectious bronchitis virus, complete genome | NC_001451.1 |
| Rhabdoviridae | Rice yellow stunt virus, complete genome | NC_003746.1 |
| | Northern cereal mosaic virus, complete genome | NC_002251.1 |
| | Vesicular stomatitis virus, complete genome | NC_001560.1 |
| | Spring viremia of carp virus, complete genome | NC_002803.1 |
| | Bovine ephemeral fever virus, complete genome | NC_002526.1 |
| | Viral hemorrhagic septicemia virus, complete genome | NC_000855.1 |
| | Rabies virus, complete genome | NC_001542.1 |
| | Snakehead rhabdovirus, complete genome | NC_000903.1 |
| | Infectious hematopoietic necrosis virus, complete genome | NC_001652.1 |
| | Sonchus yellow net virus | NC_001615.1 |
| | Australian bat lyssavirus, complete genome | NC_003243.1 |
| Filoviridae | Marburg virus, complete genome | NC_001608.2 |
| | Ebola virus, complete genome | NC_002549.1 |
| Paramyxovirinae | Mumps virus, complete genome | NC_002200.1 |
| | Sendai virus, complete genome | NC_001552.1 |
| | Measles virus, complete genome | NC_001498.1 |
| | Human parainfluenza virus 1 strain Washington/1964, | NC_003461.1 |
| | complete genome | |
| | Newcastle disease virus, complete genome | NC_002617.1 |
| | Human parainfluenza virus 3, complete genome | NC_001796.2 |
| | Human parainfluenza virus 2, complete genome | NC_003443.1 |
| | Nipah virus, complete genome | NC_002728.1 |
| | Avian paramyxovirus 6, complete genome | NC_003043.1 |
| | Bovine parinfluenza virus 3, complete genome | NC_002161.1 |
| | Hendra virus, complete genome | NC_001906.1 |
| | Canine distemper virus, complete genome | NC_001921.1 |
| | Tupaia paramyxoviurs, complete genome | NC_002199.1 |
| Orthomyxoviridae | Influenza A virus RNA segment 1, complete sequence | NC_002023.1 |
| | Influenza A virus RNA segment 3, completed sequence | NC_002022.1 |
| | Influenza A virus RNA segment 2, complete sequence | NC_002021.1 |
| | Influenza A virus RNA segment 8, complete sequence | NC_002020.1 |
| | Influenza A virus RNA segment 5, complete sequence | NC_002019.1 |
| | Influenza A virus RNA segment 6, complete sequence | NC_002018 |
| | Influenza A virus RNA segment 4, complete sequence | NC_002017.1 |
| | Influenza A virus RNA segment 7, complete sequence | NC_002016.1 |
| | Influenza B virus RNA-1, completed sequence | NC_002204.1 |
| | Influenza B virus RNA-8, complete sequence | NC_002211.1 |
| | Influenza B virus RNA-7, complete sequence | NC_002210.1 |
| | Influenza B virus RNA-6, complete sequence | NC_002209.1 |
| | Influenza B virus RNA-5, complete sequence | NC_002208.1 |
| | Influenza B virus RNA-4, complete sequence | NC_002207.1 |
| | Influenza B virus RNA-3, complete sequence | NC_002206.1 |
| | Influenza B virus RNA-2, complete sequence | NC_002205.1 |
| Bunyaviridae | Watermelon spotted wilt segment S, complete sequence | NC_003843.1 |
| | Watermelon spotted wilt virus segment M, complete | NC_003841.1 |
| | sequence | |
| | Watermelon spotted wilt virus segment L, complete | NC_003832.1 |
| | sequence | |
| | Impatiens necrotic spot virus segment L, complete sequence | NC_003625.1 |
| | Impatiens necrotic spot virus segment S, complete sequence | NC_003624.1 |
| | Peanut bud necrosis virus segment M, complete sequence | NC_003620.1 |
| | Peanut bud necrosis virus segment S, complete sequence | NC_003619.1 |
| | Impatiens necrotic spot virus segment M, complete sequence | NC_003616.1 |
| | Peanut bud necrosis virus segment L, complete sequence | NC_003614.1 |
| | Rift Valley fever virus L segment, complete sequence | NC_002043.1 |
| | Bunyamwera virus L segment, complete sequence | NC_001925.1 |
| | Andes virus segment L, complete sequence | NC_003468.1 |
| | Andes virus segment M, complete sequence | NC_003467.1 |
| | Andes virus segment S, complete sequence | NC_003466.1 |
| | Tomato spotted wilt virus RNA-L, complete sequence | NC_002052.1 |
| | Tomato spotted wilt virus RNA-S, completed sequence | NC_002051.1 |
| | Tomato spotted wilt virus RNA-M, complete sequence | NC_002050.1 |
| | Rift Valley fever virus S segment, complete sequence | NC_002045.1 |
| | Rift Valley fever virus M, segment, complete sequence | NC_002044.1 |
| | Bunyamwera virus S segment, complete sequence | NC_001927.1 |
| | Bunyamwera virus M segment, complete sequence | NC_001926.1 |
| Arenaviridae | Ippy virus nucleocapsid protein gene, parital cds | IVU80003.1 |
| Lassa | Lassa virus glycoprotein precursor (GP) and nucleoprotein | AF333969.1 |
| | (NP) genes, complete cds | |
| | Lassa virus partial genomoic RNA for putative glycoprotein | AJ310764.1 |
| | precursor (gpc gene), isolate | |
| | Lassa virus strain AV glycoprotein precursor (GPC) and | AF246121.1 |
| | nucleoprotein (NP) genes, complete cds | |
| | Lassa virus strain las9608911 nucleoprotein gene, partial cds | AF182272.1 |
| | Lassa virus strain las803796 nucleoprotein gene, partial cds | AF182271.1 |
| | Lassa virus strain las808255 nucleoprotein gene, partial cds | AF182270.1 |
| | Lassa virus strain las807868 nucleoprotein gene, partial cds | AF182269.1 |
| | Lassa virus strain las807977 nucleoprotein gene, partial cds | AF182268.1 |
| | Lassa virus strain las807992 nuceloprotein gene, partial cds | AF182267.1 |
| | Lassa virus strain las803203 nucleoprotein gene, partial cds | AF182266.1 |
| | Lassa virus strain las807998 nucleoprotein gene, partial cds | AF182265.1 |
| | Lassa virus strain las806829 nucleoprotein gene, partial cds | AF182264.1 |
| | Lassa virus strain las803793 nucleoprotein gene, partial cds | AF182263.1 |
| | Lassa virus strain las803791 nuceloprotein gene, partial cds | AF182262.1 |
| | Lassa virus strain las806828 nucleoprotein gene, partial cds | AF182262 |
| | Lassa virus strain las803792 nucleoprotein gene, partial cds | AF182260 |
| | Lassa virus strain las803201 nucleoprotein gene, partial cds | AF182259.1 |
| | Lassa virus strain las803204 nuceloprotein gene, partial cds | AF182258.1 |
| | Lassa virus strain las807974 nucleoprotein gene, partial cds | AF182257.1 |
| | Lassa virus strain las803972 nucleoprotein gene, partial cds | AF182256 |
| Reoviridae | Rice dwarf virus segment 12, complete sequence | NC_003768.1 |
| | Rice dwarf virus segment 11, complete sequence | NC_003767.1 |
| | Rice dwarf virus segment 6, complete sequence | NC_003763.1 |
| | Rice dwarf virus segment 5, complete sequence | NC_003762.1 |
| | Rice dwarf virus segment 4, complete sequence | NC_003761.1 |
| | Rice dwarf virus segment 7, complete sequence | NC_003760.1 |
| | Rice dwarf virus segment 2, complete sequence | NC_003774.1 |
| | Rice dwarf virus segment 1, complete sequence | NC_003773.1 |
| | Rice dwarf virus segment 3, complete sequence | NC_003772.1 |
| | Rice ragged stunt virus segment 4, complete sequence | NC_003771.1 |
| | Rice ragged stunt virus segment 7, complete sequence | NC_003770.1 |
| | Rice ragged stunt virus segment 10, complete sequence | NC_003769.1 |
| | Rice ragged stunt virus segment 5, complete sequence | NC_003759.1 |
| | Rice ragged stunt virus segment 8, complete sequence | NC_003758.1 |
| | Rice ragged stunt virus segment 9, complete sequence | NC_003757.1 |
| | Rice ragged stunt virus segment 6, complete sequence | NC_003752.1 |
| | Rice ragged stunt virus segment 3, complete sequence | NC_003751.1 |
| | Rice ragged stunt virus segment 2, complete sequence | NC_003750.1 |
| | Rice ragged stunt virus segment 1, complete sequence | NC_003749.1 |
| | Rice black streaked dwarf virus segment 6, complete | NC_003737.1 |
| | sequence | |
| | Rice black streaked dwarf virus segment 5, complete | NC_003736.1 |
| | sequence | |
| | Rice black streaked dwarf virus segment 4, complete | NC_003735.1 |
| | sequence | |
| | Rice black streaked dwarf virus segment 2, complete | NC_003734.1 |
| | sequence | |
| | Rice streaked dwarf virus segment 10, complete sequence | NC_003733.1 |
| | Rice black streaked dwarf virus segment 8, complete | NC_003732.1 |
| | sequence | |
| | Rice black streaked dwarf virus segment 9, complete | NC_003731.1 |
| | sequence | |
| | Rice black streaked dwarf virus segment 7, complete | NC_003730.1 |
| | sequence | |
| | Rice black streaked dwarf virus segment 1, complete | NC_003729.1 |
| | sequence | |
| | Rice black streaked dwarf virus segment 3, complete | NC_003728.1 |
| | sequence | |
| | Eyach virus segment 12, complete sequence | NC_003707.1 |
| | Eyach virus segment 11, complete sequence | NC_003706.1 |
| | Eyach virus segment 10, complete sequence | NC_003705.1 |
| | Eyach virus segment 9, complete sequence | NC_003704.1 |
| | Eyach virus segment 8, complete sequence | NC_003703.1 |
| | Eyach virus segment 7, complete sequence | NC_003702.1 |
| | Eyach virus segment 6, complete sequence | NC_003701.1 |
| | Eyach virus segment 5, complete sequence | NC_003700.1 |
| | Eyach virus segment 4, complete sequence | NC_003699.1 |
| | Eyach virus segment 3, complete sequence | NC_003698.1 |
| | Eyach virus segment 2, complete sequence | NC_003697.1 |
| | Eyach virus segment 1, complete sequence | NC_003696.1 |
| | Nilaparvata lugens reovirus segment 9, complete sequence | NC_003661.1 |
| | Nilaparvata lugens reovirus segment 7, complete sequence | NC_003660.1 |
| | Nilaparvata lugens reovirus segment 6, complete sequence | NC_003659.1 |
| | Nilapravata lugens reovirus segment 5, complete sequence | NC_003658.1 |
| | Nilaparvata lugens reovirus segment 4, complete sequence | NC_003657.1 |
| | Nilaparvata lugens reovirus segment 3, complete sequence | NC_003656.1 |
| | Nilaparvata lugens reovirus segment 2, complete sequence | NC_003655.1 |
| | Nilaparvata lugens reovirus segment 1, complete sequence | NC_003654.1 |
| | Nilaparvata lugens reovirus segment 8, complete sequence | NC_003653.1 |
| | Nilaparvata lugens reovirus segment 10, complete sequence | NC_003652.1 |
| | Lymantria dispar cypovirus 1 segment 10, complete | NC_003025.1 |
| | sequence | |
| | Lymantria dispar cypovirus 1 segment 9, complete sequence | NC_003024.1 |
| | Lymantria dispar cypovirus | NC_003023.1 |
| | 1 segment 8, complete sequence | |
| | Lymantria dispar cyporvirus 1 segment 7, complete virus | NC_003022.1 |
| | Lymantria dispar cyporvirus 1 segment 6, complete sequence | NC_003021.1 |
| | Lymantria dispar cypovirus | NC_003020.1 |
| | 1 segment 5, complete sequence | |
| | Lymantria dispar cyporivus 1 segment 4, complete sequence | NC_003019.1 |
| | Lymantria dispar cypovirus 1 segment 3, complete sequence | NC_003018.1 |
| | Lymantria dispar cypovirus 1 segment 2, complete sequence | NC_003017.1 |
| | Lymantria dispsar cypovirus 1 segment 1, complete | NC_003016.1 |
| | sequence | |
| | Lymantria dispar cypovirus 14 segment 10, complete | NC_003015.1 |
| | sequence | |
| | Lymantria dispar cypovirus | NC_003014.1 |
| | 14 segment 9, complete sequence | |
| | Lymantria dispar cypovirus 14 segment 8, complete | NC_003013.1 |
| | sequence | |
| | Lymantria dispar cypovirus 14 segment 7, complete | NC_003012.1 |
| | sequence | |
| | Lymantria dispar cypovirus 14 segment 6, complete | NC_003011.1 |
| | sequence | |
| | Lymantria dispar cypovirus 14 segment 5, complete | NC_003010.1 |
| | sequence | |
| | Lymantria dispar cypovirus 14, segment 4, complete | NC_003009.1 |
| | sequence | |
| | Lymantria dispar cypovirus 14 segment 3, complete | NC_003008.1 |
| | sequence | |
| | Lymantria dispar cypovirus 14 segment 2, complete virus | NC_003007.1 |
| | Lymantria dispar cypovirus 14 segment 1, complete | NC_003006.1 |
| | sequence | |
| | Trichoplusia ni cytoplasmic polyhedrosis virus 15 segment | NC_002567.1 |
| | 4, complete sequence | |
| | Trichoplusia ni cytoplasmic polyhedrosis virus 15 segment | NC_002566.1 |
| | 11, complete sequence | |
| | Trichoplusia ni cytoplasmic polyhedrosis virus 15 segment | NC_002565.1 |
| | 10, complete sequence | |
| | Trichoplusia ni cytoplasmic polyhedrosis virus 15 segment | NC_002564.1 |
| | 9, complete sequence | |
| | Trichoplusia ni cytoplasmic polyhedrosis virus 15, segment | NC_002563.1 |
| | 8, complete sequence | |
| ** | Trichoplusia ni cytoplasmic polyhedrosis virus 15 segment | NC_002562.1 |
| | 7, complete sequence | |
| | Trichoplusia ni cytoplasmic polyhedrosis virus 15 segment | NC_002561.1 |
| | 6, complete sequence | |
| | Trichoplusia ni cytoplasmic polyhedrosis virus 15 segment | NC_002560.1 |
| | 5, complete sequence | |
| | Trichoplusia ni cytoplasmic polyhedrosis virus 15 segment | NC_002559.1 |
| | 3, complete sequence | |
| | Trichoplusia ni cytoplasmic polyhedrosis virus 15, segment | NC_002558.1 |
| | 2, complete sequence | |
| | Trichoplusia ni cytoplasmic polyhedrosis virus 15, segment | NC_002557.1 |
| | 1, complete sequence | |
| Prion | Homo sapiens mRNA for prion protein, complete cds | D00015.1 |
| | Human prion protein 27-30 mRNA, complete cds | M13667 |
| | Homo sapiens prion protein (p27-30) (Creutzfled-Jakob | NM_000311 |
| | disease, Gerstmann-Strausler-Scheinker syndrome, fatal | |
| | familial insomnia) (PRNP), mRNA | |
| | MAJOR PRION PROTEIN PRECURSOR (PRP) (PRP27-30) | P52114 |
| | (PRP33-35C) | |
| | prion protein [Mustela putorius] | AAA69022 |
| | prion protein [mink, Genomic, 2446 nt] | S46825 |
| | Major prion protein precursor (PrP) (PrP27-30) (PrP33-35C) | P04156 |
| | (ASCR) (CD230 antigen) | |
| | Odocoileus virginianus prion protein precursor (PrP) gene, | AF156185 |
| | complete cds | |
| | Odocoileus virginianus prion protein precursor (PrP) gene, | AF156186 |
| | PrP-96Gly-138Asn allele, partial cds | |
| | Cervus elaphus nelsoni prion protein precursor (PrP) gene, | AF156182 |
| | PrP-132L allele, complete cds | |
| | Antilocapra Americana prion protein precursor (PrP) gene, | AF156187 |
| | complete cds | |
| | Cervus elaphus nelsoni prion protein precursor (PrP) gene, | AF156183 |
| | complete cds | |
| | Odocoileus virginianus prion protein precursor (PrP) gene, | AF156184 |
| | PrP-96Ser allele, complete cds | |
| | Felis catus prion protein (Prp) gene, complete cds | AF003087.1 |
| | Sheep gene for protein PrP, complete cds | D38179.1 |
| | Bos taurus prp gene for prion protein | AJ298878 |
| | Bos taurus mRNA for prion protein, complete cds | AB001468 |
| | Bovine mRNA for prion protein | D10612 |
| | Capra hircus prion protein (PrP) gene, complete cds | S82626 |
| | Homo sapiens v-abl Abelson murine leukemia viral | XM_033355.1 |
| | oncogene homology 1 | |
| | Mus musculus similar to Proto-oncogene tyrosine-protein | XM_130089 |
| | kinase ABL1 (p150) (c-ABL) (LOC227716), mRNA | |
| B-Raf | Homo sapiens v-raf murine sarcoma viral oncogene | NM_004333 |
| | homology B1 (BRAF), mRNA | |
| | H. sapiens B-raf-1 gene for 94 kDa B-raf protein | X65187.1 |
| | Mus musculus similar to B-Raf proto-oncogene | XM_133086 |
| | serin/threoine-protein kinase (p94) (v-Raf murine sarcoma | |
| | viral oncogene homolog B1) (LOC232705), mRNA | |
| | Mus musculus WGS supercontig Mm6_WIFeb01_98 | NW_000273 |
| BCL1 | H. sapiens of BCL1 mRNA encoding cyclin | Z23022 |
| | Homo sapiens genomic DNA, chromosome 11q, clone: | AP001824 |
| | CTD-2507F7, complete sequence | |
| | H. sapiens cyclin D1 gene promoter region | Z29078 |
| BCL-2 | Homo sapiens BCL-2 antagonist of cell death (BAD) | NM_004322 |
| | transcript variant 1, mRNA | |
| | Homo sapiens Bcl-X/Bcl-2 binding protein (BAD) mRNA, | AF021792 |
| | partial cds | |
| | Homo sapiens v-raf-1 murine leukemia viral oncogene | NM_002880.1 |
| | homolog 1 (RAF1), mRNA | |
| | Homo sapiens BCL-2 antagnoist of cell death (BAD) | NM_032989.1 |
| | transcript variant 2, mRNA | |
| BCL-6 | Human zinc-finger protein (bcl-6) mRNA, complete cds | UO0115 |
| CBFA2 | Human AML1 mRNA for AML1c protein (alternatively | XM_003789 |
| | spliced product), complete cds | |
| CSF1R | Homo sapiens colony stimulating factor 1 receptor, formerly | XM_003789 |
| | McDonough feline sarcoma viral (v-fms) oncogene | |
| | homology (CSF1R), mRNA | |
| | Homo sapiens choromosome 5 working draft segment | NT_006859 |
| EGFR | Human epidermal growth factor (beta-urogastrone) gene | J02548.1 |
| | (synthetic) | |
| | Homo sapiens epidermal growth factor (beta-urogastrone) | NM_001963.2 |
| | (EGF), mRNA | |
| ERB-B-2 | Human tyrosine kinase-type receptor (HER2) mRNA, | M11730.1 |
| | complete cds | |
| | Human c-erb-B-mRNA | X03363 |
| FOS | Human cellular oncogene c-fos (complete sequence) | V01512 |
| | Human fos proto-oncogene (c-fos), complete cds | K00650 |
| | Homo sapiens v-fos FBJ murine osteosarcoma viral | NM_005252.2 |
| | oncogene homology (FOS), mRNA | |
| HRAS | Human (genomic clones lambda-[SK2-T2-HS578T]; cDNA | J00277 |
| | clones RS-[3,4, 6]) c-Ha-ras 1 proto-oncogene, complete | |
| | coding sequence | |
| | Homo sapiens, Similar to v-Ha-ras Harvey rat sarcoma viral | BC006499.1 |
| | oncogene homology, clone MGC: 2359 IMAGE: 2819996, | |
| | mRNA, complete cds | |
| Myb | Human c-myb mRNA, complete cds | M15024.1 |
| | Homo sapiens v-myb myeloblastosis viral oncogene | NM_005375.1 |
| | homology (avian) (MYB), mRNA | |
| | Human c-myb mRNA, complete cds | M15024.1 |
| | Human (c-myb) gene, complete primary cds, and five | HSU22376.1 |
| | complete alternatively spliced cds | |
| | c-myb {promoter, 5′ region} [human, leukocytes, Genomic, | S66422.1 |
| | 1284 nt] | |
| Myc | Human mRNA encoding the c-myc oncogene | V00568.1 |
| | Homo sapiens v-myc myelocytomatosis viral oncogene | NM_002467 |
| | homolog (avian) (MYC), rRNA | |
| | Homo sapiens MYC gene for c-myc proto-oncogene and | X00364.2 |
| | ORF-1 | |
| | Human c-myc-P64 mRNA, initiating from promoter P0, | M13930.1 |
| | (Hlmyc3.1) partial cds | |
| LCK | Human lck mRNA for membrane associated protein tyrosine | X13529.1 |
| | kinase | |
| | Human lymphocyte-specific protein tyrosine kinase (lck) | M36881.1 |
| | mRNA, complete cds | |
| | Homo sapiens lymphocyte-specific protein tyrosine kinase | M26693.1 |
| | (LCK) gene, exon2 and upstream promoter region | |
| | Human mutant lymphocyte-specific protein tyrosine kinase | U07236.1 |
| | (LCK) mRNA, complete cds | |
| | Human T-lymphocyte specific protein tyrosine kinase p56lck | U23852 |
| | (lck) abberant mRNA, complete cds | |
| | homo sapiens, clone MGC: 17196 IMAGE: 4341278, mRNA, | BC013200.1 |
| | complete cds | |
| MYCL1 | Homo sapiens v-myc myelocytomatosis viral oncogene | NM_005376.1 |
| | homology, lung carcinoma derived (avian-(MYCL1), | |
| | mRNA | |
| MYCN | Homo sapiens v-myc myelocytomatosis viral related | NM_005378 |
| | oncogene, neuroblastoma derived (avian_(MYCN), mRNA | |
| | Homo sapiens truncated MYCN fusion protein (MYCN) | AF317388 |
| | gene, complete cds | |
| NRAS | Rattus norvegicus Neuroblastoma RAS viral (v-ras_oncogene | NM_080766.1 |
| | homolog (Nras), mRNA | |
| | Mus musculus WGS supercontig Mm3_WIFeb01_50 | NW_000200.1 |
| | Mus musculus neuroblastoma ras oncogene (Nras), mRNA | XM_124137.1 |
| | Homo sapiens chromosome 1 working draft sequence | NT_019273.11 |
| | segment | |
| | Homo sapiens neuroblastoma RAS viral (v-ras) oncogene | XM_032698.6 |
| | homolog (NRAS), mRNA | |
| | G-15 Mouse, 4 months old female, left ventricular cardiac | BM658481.1 |
| | muscle cells cDNA library Mus musculus cDNA similar to | |
| | Mus musculus neuroblastoma ras oncogene (Nras), | |
| | Homo sapiens Ras family small GTP binding protein N-Ras | AF493919.1 |
| | (NRAS) mRNA, complete cds | |
| ROSI | Mus musculus Ros1 proto-oncogene (Ros1), mRNA | XM_125632 |
| | Homo sapiens chromosome 6 working draft sequence | NT_033944 |
| | segment | |
| | Homo sapiens v-ros UR2 sarcoma virus oncogene homolog | NM_002944.2 |
| | 1 (avian) (ROS1), mRNA | |
| | Human c-ros-1 proto-oncogene | AH002964.1 |
| RET | RET = proto-oncogene [human, neuroblastoma cell line LA- | S80097 |
| | 0N-2, 3621 nt] | |
| | Homo sapiens v-src sarcoma (Schmidt-Ruppin A-2) viral | NM_005417.2 |
| | oncogene homolog (avian) | |
| TCF3 | Human transcription factor (ITF-1) mRNA, 3′ end | X52078.1 |
| | Human e12 protein (E2A) mRNA, complete cds | M31222 |
| | Human (HeLa) helix-loop-helix protein HE47 (E2A) | M65214.1 |
| | mRNA, 3′ end | |
| | Human transcription factor (E2A) mRNA, complete cds | M31523.1 |

T7 Promoter/T7 Polymerase Expression Systems

A desirable method of the invention utilizes a T7 dsRNA expression system to achieve cytoplasmic expression of dsRNA, (e.g., long or short dsRNA molecules) in vertebrate cells (e.g., mammalian cells). Intracellular expression of short dsRNA molecules is expected to increase the duration of the silencing with respect to exogenously added short dsRNA molecules. The T7 expression system utilizes the T7 promoter to express the desired dsRNA. Transcription is driven by the T7 RNA polymerase, which can be provided on a second plasmid or on the same plasmid. For example, a first plasmid construct that expresses both a sense and antisense strand under the control of converging T7 promoters and a second plasmid construct that expresses the T7 RNA polymerase under the control of an RSV promoter can be used. Both the dsRNA and the T7 RNA polymerase could advantageously be expressed from a single bicistronic plasmid construct, particularly when the dsRNA is formed from a single RNA strand with inverted repeats or regions of self-complementarity that enable the strand to assume a stem-loop or hairpin structure with an at least partially double-stranded region. Individual sense and antisense strands which self assemble to form a dsRNA can be synthesized by a single plasmid construct using, e.g., converging promoters such as bacteriophage T7 promoters placed respectively at the 5′ and 3′ ends of the complementary strands of a selected sequence to be transcribed.

### Example 4

### Exemplary Methods for the Generation of dsRNA In Vitro

Short and long dsRNA can be made using a variety of methods known to those of skill in the art. For example, ssRNA sense and antisense strands, or single RNA strands with inverted repeats or regions of self-complementarity that enable the strand to assume a stem-loop or hairpin structure with an at least partially double-stranded region, including the hairpin structures of the invention, can be synthesized chemically in vitro (see, for example, Q. Xu et al, Nucl. Acids. Res., 24 (18): 3643-3644, 1996 and other references cited in WO 00/63364, pp. 16-7), transcribed in vitro using commercially available materials and conventional enzymatic synthetic methods, (e.g., using the bacteriophage T7, T2, or SP6 RNA polymerases according to conventional methods such as those described by Promega Protocols and Applications Guide 3^{rd} Ed., Eds. Doyle, 1996, ISBN No. 1-882274-57-1), or expressed in cell culture using recombinant methods. The RNA can then be purified using non-denaturing methods inducing various chromatographic methods and hybridized to form dsRNA. Such methods are well known to those of skill in the art and are described, for example, in WO 01/75164, WO 00/63364, and Sambrook et al., Molecular Cloning, A Laboratory Manual, 2^{nd} Ed.; Cold Spring Harbor Laboratory Press, New York, 1989, the teaching of which is incorporated herein by reference.

In vitro transcription reactions can be carried out using the Riboprobe Kit (Promega Corp.), according to the manufacturer's directions. The template DNA is as described above. Following synthesis, the RNA is treated with Proteinase K and extracted with Phenol-chloroform to remove contaminating RNases. The RNA is ethanol precipitated, washed with 70% ethanol, and resuspended in RNase-free water. Aliquots of RNA are removed for analysis and the RNA solution is flash frozen by incubating in an ethanol-dry ice bath. The RNA is stored at −80° C.

As an alternative to phenol-chloroform extraction, RNA can be purified in the absence of phenol using standard methods such as those described by Li et al. (WO 00/44943, filed Jan. 28, 2000). Alternatively, RNA that is extracted with phenol and/or chloroform can be purified to reduce or eliminate the amount of phenol and/or chloroform. For example, standard column chromatography can be used to purify the RNA (WO 00/44914, filed Jan. 28, 2000).

Double-stranded RNA can be made by combining equimolar amounts of PCR fragments encoding antisense RNA and sense RNA, as described above, in the transcription reaction. Single-stranded antisense or sense RNA is made by using single species of PCR fragments in the reaction. The RNA concentration is determined by spectrophotometric analysis, and RNA quality is assessed by denaturing gel electrophoresis and by digestion with RNase T1, which degrades single-stranded RNA.

If desired, an mRNA library is produced using Qbeta bacteriophage, by ligating the mRNA molecules to the flank sequences that are required for Qbeta replicase function (Qbeta flank or Qbeta flank plus P1), using RNA ligase. The ligated RNA molecules are then transformed into bacteria that express Qbeta replicase and the coat protein. Single plaques are then inoculated into fresh bacteria. All plaques are expected to carry transgene sequences. Each plaque is grown in larger quantities in bacteria that produce the Qbeta polymerase, and RNA is isolated from the bacteriophage particles. Alternatively, if the Qbeta flank plus P1 is used to generate the library (e.g., P1=MS2, VEEV, or Sindbis promoter sequences), these vectors can be used to carry out the in vitro transcription along with the cognate polymerase. The in vitro made dsRNA is then used to transfect cells.

### Example 5

### Generation of Constructs Encoding Duplexes or Hairpins with Mismatches

Duplexes with Mismatches

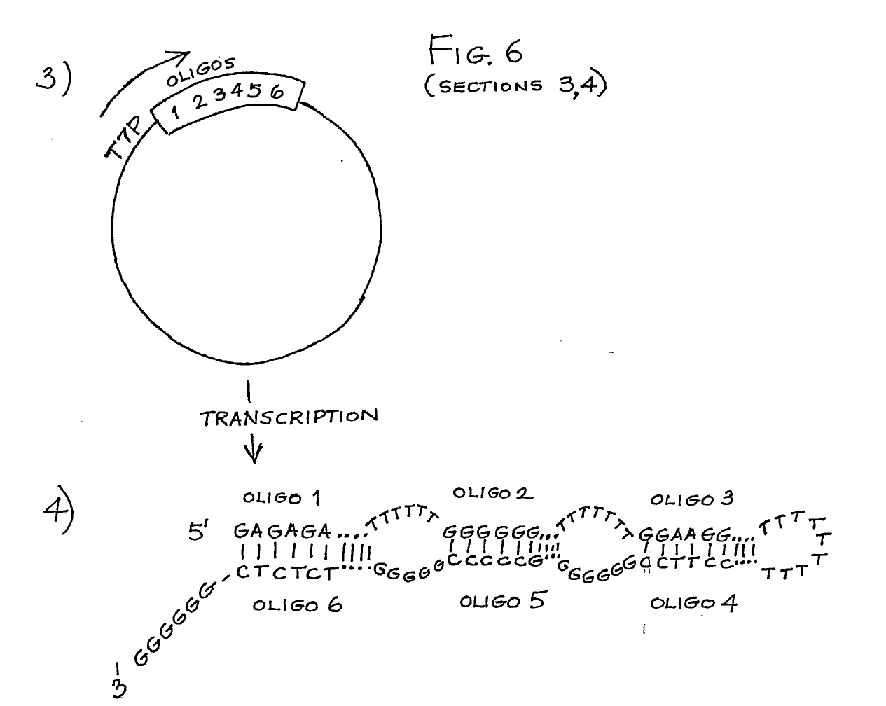

Sequences encoding large RNA duplexes with mismatched regions are cloned such that the sense and antisense RNAs are transcribed by separate cistrons. The separate cistrons may be present on the same expression vector, e.g., two cistrons on the same plasmid, or on separate expression vectors, e.g., two plasmids expressing the separate cistrons in the same cell, as shown in FIGS. 4A and 4B. FIGS. 5A and 5B outline an example by which such molecules can be constructed. The invention is not meant to be limited to this method as there are multiple ways to design such constructs and there are multiple compositions of these constructs as defined in the brief description of the invention and those skilled in the art would easily be able to generate these constructs using a variety of methods. Briefly, for sense strand synthesis, oligonucleotides derived from a target sequence are synthesized in such a way that there is a stretch of mismatched sequences located at the end of the oligonucleotides. This stretch of sequence is designed to be mismatched with the antisense RNA strand of the RNA duplex (not to be confused with complementary oligonucleotides). In FIG. 5A(1), the mismatched sequences are confined to Box B of each depicted oligonucleotide. In the depicted example, Box B encodes a string of 7 T residues on the top oligonucleotide while the complementary oligonucleotide contains a string of 7 A residues. Box A of each oligonucleotide desirably contains at least 18 to 19 nucleotides derived from at least 19 contiguous nucleotides of the target sequence. Desirably, a sequence of 18 to 30, 19 to 30, preferably 19 to 27, 20 to 26, 21 to 25, 21 to 24, or 21 to 23 nucleotides having sequence identity with a target polynucleotide will be included in each oligonucleotide. However, the number of such nucleotides designed to be in double-stranded conformation, and the selected target sequence, may vary from oligonucleotide to oligonucleotide, i.e., 21 nucleotides in oligo 1, 23 nucleotides in oligo 2, etc.). In FIG. 5A(2), three different ds-oligonucleotides are synthesized, oligonucleotides 1, 2 and 3. Each oligonucleotide is annealed to its counterpart complementary oligonucleotide. Following annealing of each oligonucleotide pair, the three annealed oligonucleotides are directionally ligated as indicated to generate the product shown in FIG. 5A(3). The product can be directly directionally ligated into a chosen vector or it can first be PCR amplified and the PCR product directionally ligated into a chosen vector. Directional ligation is performed as described in U.S. Pat. No. 6,143,527, Pachuk and Satishchandran, “Chain Reaction Cloning Using a Bridging Oligonucleotide and DNA Ligase”, and in “Chain reaction cloning: a one-step method for directional ligation of multiple DNA fragments”, Pachuk et al., Gene, 243 pp 19-25, 2000. The use of directional ligation is discussed later.

For antisense strand synthesis (FIG. 5B), the strategy is similar to sense strand synthesis except that Box B of each oligonucleotide encodes nucleotides for the antisense RNA strand that are designed not to basepair with Box B derived nucleotides present in the sense RNA strand (see, e.g., FIG. 5A). The DNA encoding the antisense strand is cloned directionally into a vector such that the antisense strand is transcribed. The antisense strand will include a series of sequences designed to basepair with the sense strand (to form the double-stranded regions) separated by mismatched regions designed to remain single-stranded. The sense and antisense strands are therefore cloned as separate cistrons (transcription units). These cistrons can be in the same vector or separate vectors. Following transcription of each cistron in the same cell, the antisense and sense RNA strands anneal to each other, generating a large RNA duplex with double-stranded regions separated by mismatches. Accordingly, the RNA duplex will exist as regions of dsRNA interspersed with regions in which the RNA sequences of the sense and antisense strands are non-complementary and do not form a double-stranded structure. As little as a single nucleotide insertion or deletion in one of the two strands will serve as such a mismatch, and because of steric constraints governing basepairing, will result in a “bubble” of 4 non-basepaired nucleotides; however, a mismatch of two, three or four or more nucleotides are desirable in certain embodiments. In desirable embodiments, there will be between about 4 and 10 nucleotides, about 4 to 20, about 4 to 50, or about 4 to about 100 nts in a mismatched region. In some embodiments, a mismatched region may include more than 100 nts, e.g., several hundred to a thousand nts. Mismatched regions, particularly longer mismatched regions, may themselves include stem-loop or other structures.

Directional Ligation

Directional ligation is important to ensure the proper positioning in the duplex of sense sequences with respect to antisense sequences. This enables the proper alignment of these sequences with respect to each other and facilitates basepairing of sense sequences with antisense sequences in the resulting duplex RNA. In the example shown in FIG. 5 A, oligos 1, 2, and 3 are directionally ligated to create the sequence order of 1, 2, 3 (or 3, 2, 1). This is important only with respect to creating a sequence that can basepair with the designed complementary RNA strand, which in this example is 4, 5, 6 or (6, 5, 4). One could also arrange in other orders such as 2, 3, 1 if one also ligates the other oligo set in the respective order of 5, 6, 4. Likewise oligos representing sense and antisense polarity can be ligated such that each strand of the resultant duplex is a mix of antisense and sense sequences with respect to the target RNA so long as each of the strands can basepair with the other except for those regions (Box B) designed to be mismatched and not able to basepair. Directional ligation is also useful for ligation of the inserted sequences into the vector of choice. This is to ensure that a specific polarity only is transcribed as indicated in FIGS. 5A and 5B.

RNA Hairpins with Mismatches

RNA hairpins are unimolecular structures and therefore sequences encoding the sense and antisense RNA sequences (with respect to the target RNA) are cloned such that transcription of these sequences is from a single promoter. The resultant molecule is predicted to adopt a hairpin structure with regions of mismatch such as the molecule depicted in FIGS. 2A and 2B. FIG. 6 describes a method for generating large RNA hairpins with mismatches. The invention is not meant to be limited to this method as there are multiple ways to design such constructs and there are multiple compositions of these constructs as defined in the brief description of the invention and those skilled in the art would easily be able to generate these constructs using a variety of methods. Briefly, oligonucleotides containing Box A and Box B sequences are designed, as described above, and as shown in FIGS. 5A and 5B. Following annealing of oligonucleotides in each oligo pair, the oligonucleotides are directionally ligated in a fashion that yields a hairpin RNA. The ligated oligo can be cloned directly or subjected to PCR amplification, and the amplified product is cloned into a vector. Cloning can be in either orientation with respect to the promoter (see FIG. 6).

Structured RNAs

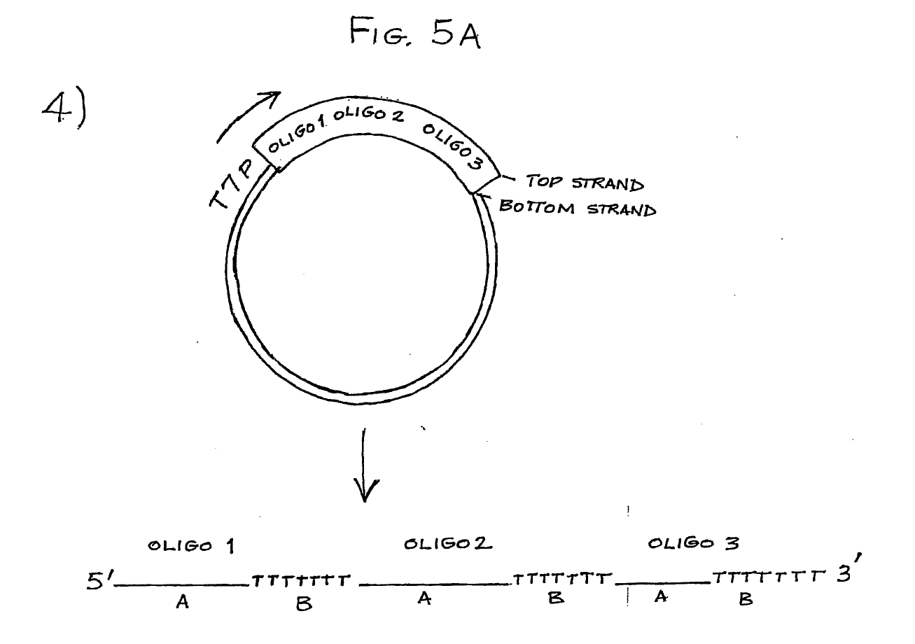

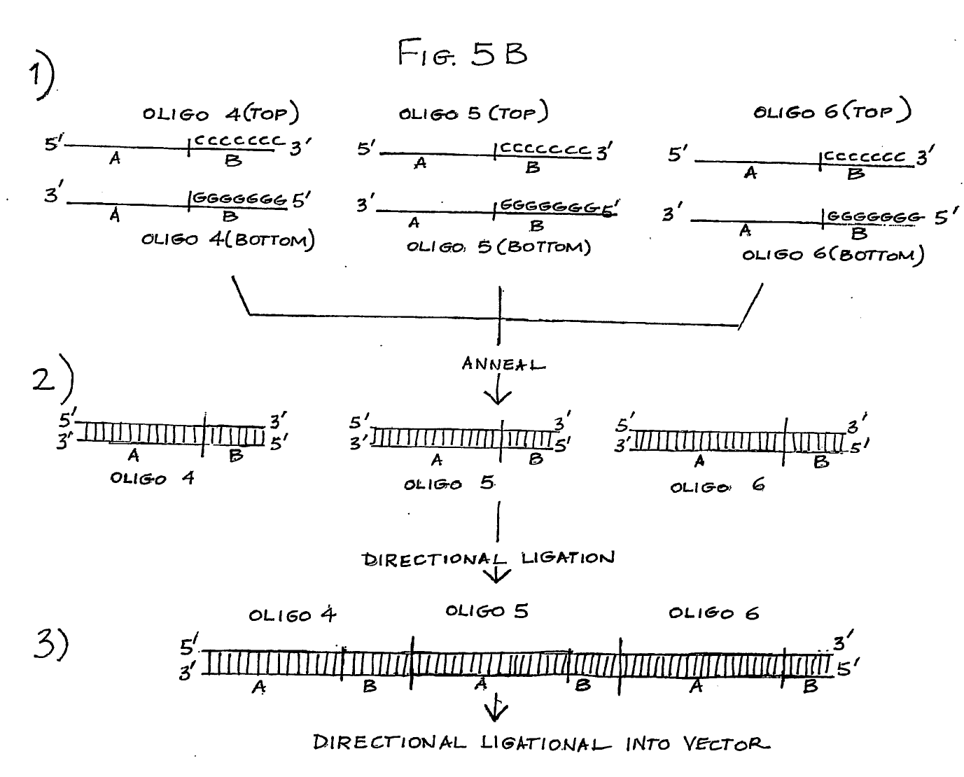

According to this embodiment, an RNA molecule is designed to contain multiple short hairpin-loop structures situated in tandem but separated from one another by at least one nucleotide, desirably 2-7 nucleotides and desirably a maximum 50 nucleotides. An example of such an RNA molecule is depicted in FIG. 3A. FIG. 7 illustrates a method for generating an RNA molecule with this type of structure. The invention is not meant to be limited to this method as there are multiple ways to design such constructs and there are multiple compositions of these constructs as defined in the brief description of the invention and those skilled in the art would easily be able to generate these constructs using a variety of methods. Briefly, oligonucleotides encoding a hairpin-loop are ligated to each other as described in FIG. 7. Some of these oligonucleotides contain one or more nucleotides located at one or both ends of some or all of the oligonucleotides. These nt(s) do not encode nt(s) that participate in the hairpin or loop structure of the encoded RNA but rather serve as the spacer nt(s) between each hairpin-loop. These spacers are important and act as processing sites by the cell's single-strand specific RNAses. Processing at these sites yields individual small duplexes of RNA as shown in FIG. 3B.

Oligonucleotides can be derived from different regions of the same or different RNAs, such that one duplex, hairpin, or “udder-structured” or “udderly structured” RNA can target one or more RNAs. 2) The number of base paired segments is minimally two and can maximally be several thousand. A desirable number is 5-500 nucleotides, inclusive.

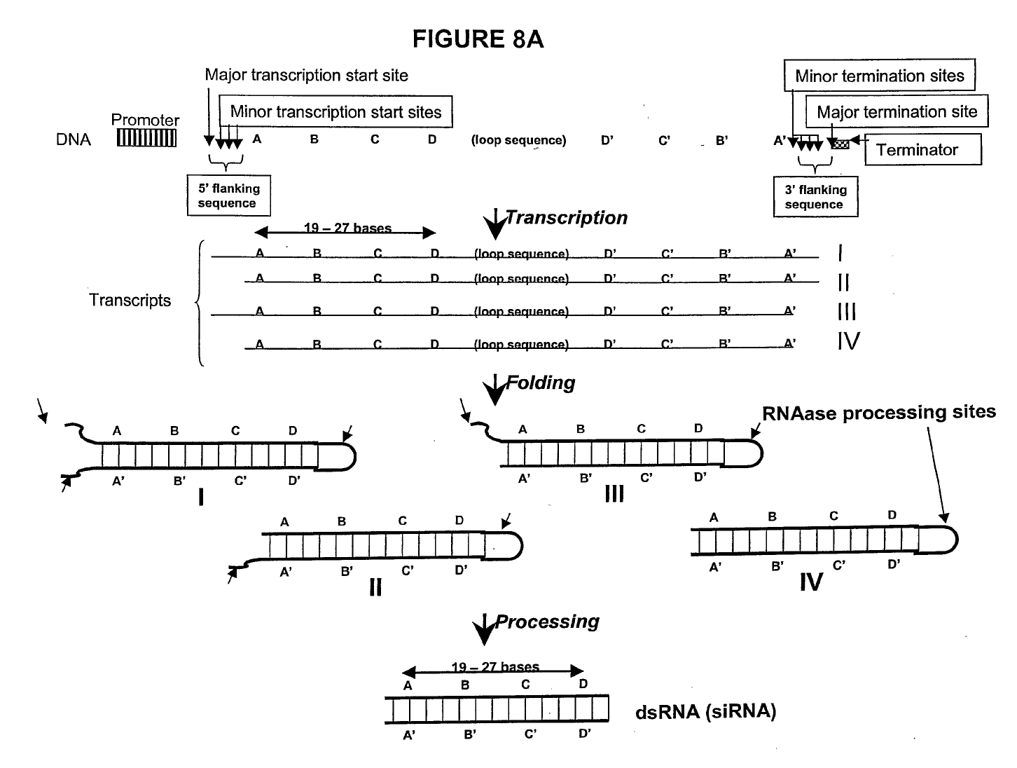

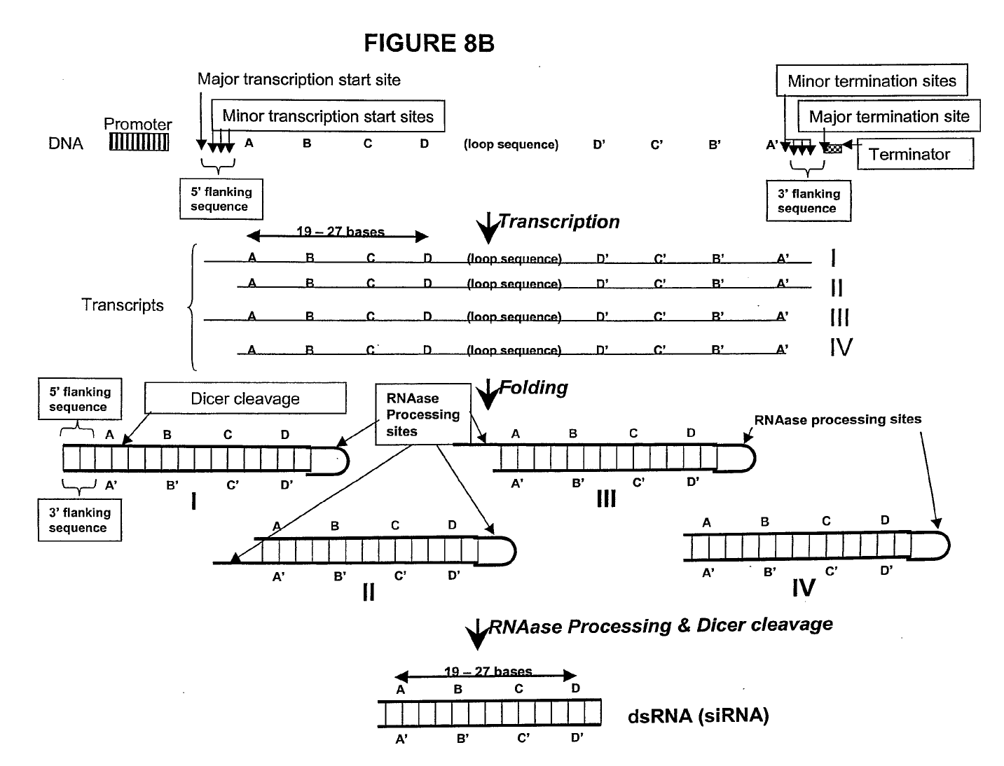

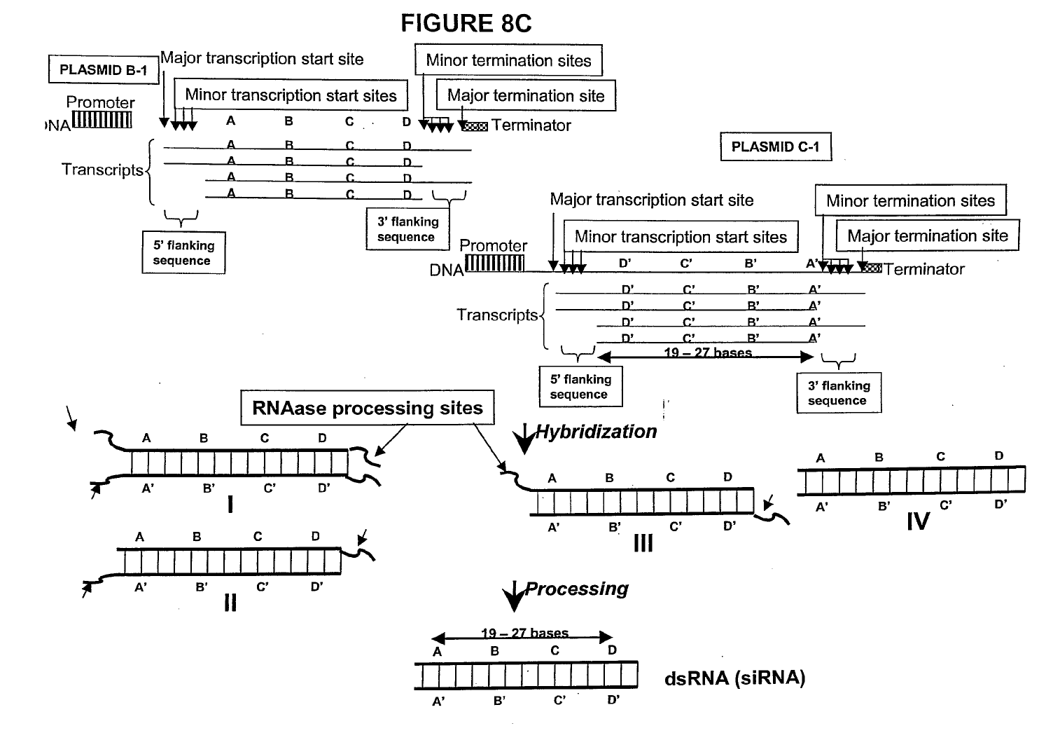

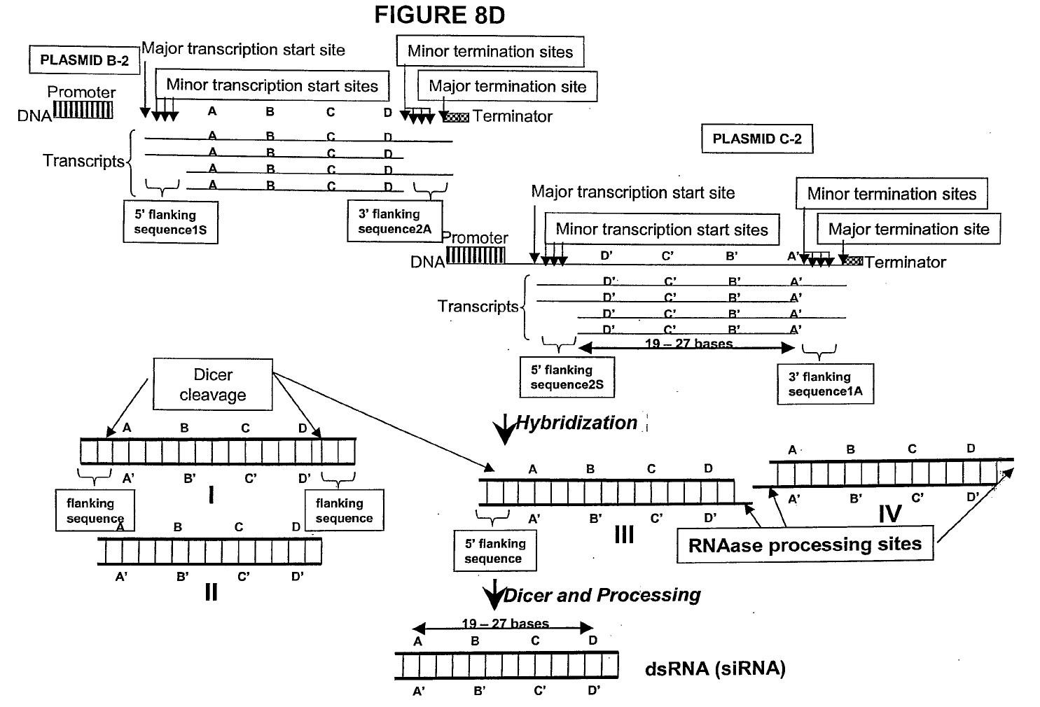

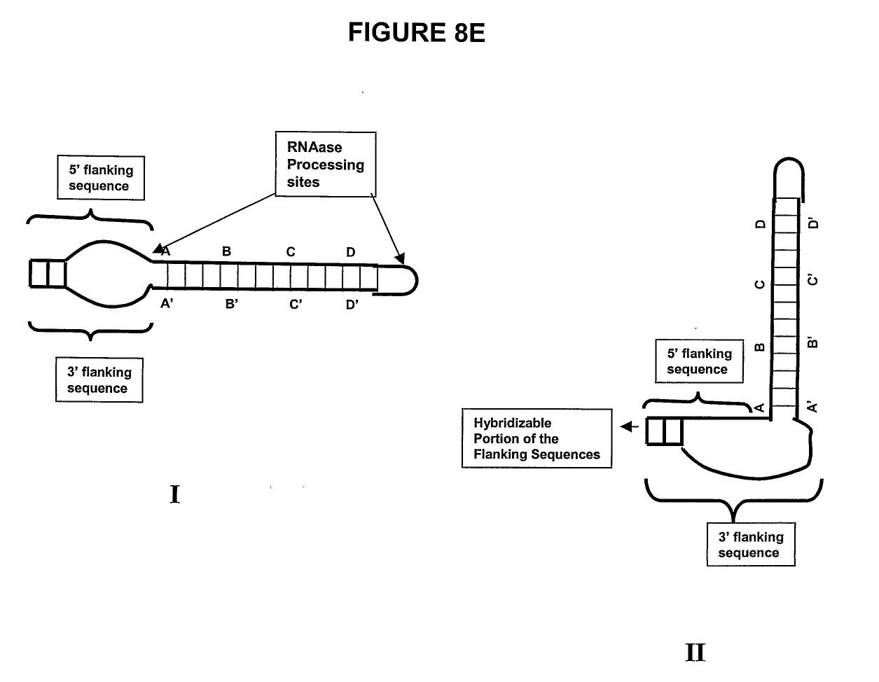

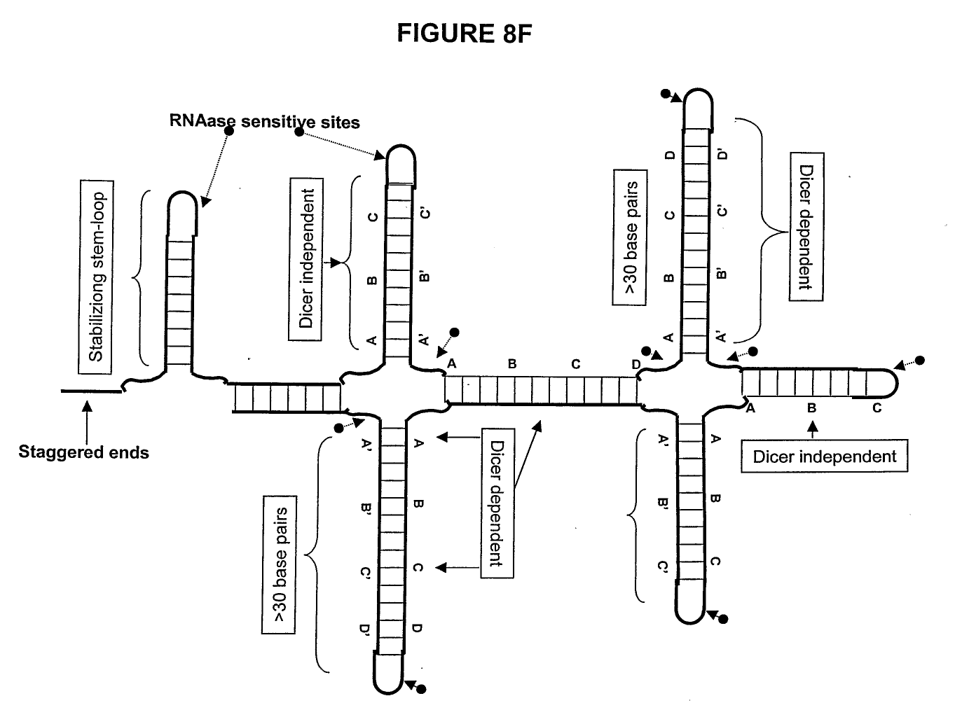

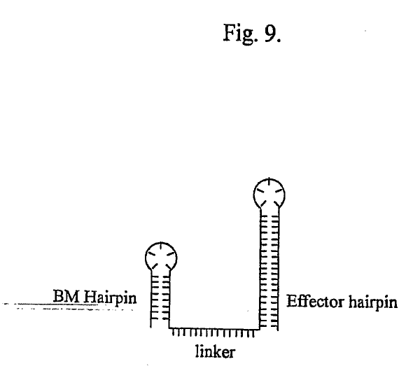

The following examples describe the construction of dsRNA constructs comprising multiple short hairpins or stem-loop structures interspersed with single-stranded “spacer” regions. The same methods may be used to construct multiple long and/or short hairpin structures, including such structures as depicted in FIG. 5F, which comprise strings of stem-loop or hairpin structures interspersed by double-stranded regions. Some of the stem-loop or hairpins are designed to enhance stability from exonucleases. For example, as seen in FIG. 5F, a stem-loop structure located in the 5′-most portion of the RNA molecule, e.g., a Bernie Moss hairpin as described in more detail in Example 10 and depicted in FIG. 9, may serve to protect the transcript, including downstream effector portions of the molecule, from degradation. The construct of FIG. 8F also includes a 5′ initiation sequence as described in Example 9. The dsRNA constructs may be “Dicer independent”, e.g., the double-stranded stem regions may be 19 to about 30 basepairs in length, so that cleavage of the single-stranded regions by single-strand cellular RNAases yields dsRNAs of 19 to 30 bp, without any cleavage by Dicer or similar enzymes. Such siRNAs (short interfering RNAs) or “sequitopes” are contiguous sequences of double-stranded polyribonucleotides that can associate with and activate RISC (RNA-induced silencing complex), usually a contiguous sequence of between 19 and 27 basepairs, e.g., 21 to 23, or 19 to 30 bp, inclusive. The dsRNA constructs may also be “Dicer dependent”, e.g., the double-stranded stem regions may be greater than about 27 to 30 basepairs in length, so that cleavage of the single-stranded regions by single-strand RNAases yields dsRNAs of greater than about 27 to about 30 basepairs, so that further dsRNA cleavage by Dicer or similar enzymes is necessary for formation of siRNAs capable of associating with and activating the RISC complex. As shown in FIG. 8F, the sequences separating the stem-loop structures may be double-stranded. The “shoulder” regions comprising the several nucleotides between the stem-loop structures and the double-stranded separating regions will include a region of at least about 4 nts, more if so desired, that will be single-stranded and will be amenable to cleavage by single-strand RNAases. If the double-stranded separating sequences comprise regions of substantial sequence homology to a target polynucleotide, e.g., at least 19 to 30 contiguous basepairs (desirably, no greater than about 200 basepairs, preferably, no greater than about 50 basepairs), they can also be cleaved to produce additional dsRNAs capable of inducing inhibition or silencing of a target. As seen in FIG. 8F, a single such structure can easily be engineered to include both Dicer-dependent and Dicer-independent double-stranded regions.

### Example 5A

### Reducing or Inhibiting the Function of HIV p24 in Virally Infected Cells

During the course of HIV infection, the viral genome is reverse transcribed into a DNA template that is integrated into the host chromosome of infected dividing cells. The integrated copy is now a blueprint from which more HIV particles are made. If the function of a polynucleotide sequence essential to replication and/or pathogenesis of HIV is reduced or inhibited, the viral infection can be treated. This example demonstrates the performance of one embodiment of the method of this invention.

Several cell lines that contain integrated copies of a defective HIV genome, HIVgpt (strain HXB2) have been created. The HIVgpt genome contains a deletion of the HIV envelope gene; all other HIV proteins are encoded. The HIVgpt genome encodes a mycophenolic acid (MPA) resistance gene in place of the envelope gene and thereby confers resistance to MPA. Cells resistant to MPA were clonally amplified. The plasmid used to create these cell lines, HIVgpt, was obtained from the AIDS Research and Reference Reagent Program Catalog. Stably integrated cell lines were made with human rhabdomyosarcoma (RD) and Cos7 cell lines. The lines were made by transfecting cells with the HIVgpt plasmid followed by selection of cells in mycophenolic acid. Cells resistant to MPA were clonally amplified using standard procedures. The media from the cultured clonally expanded cells was assayed for the presence of p24 (an HIV gag polypeptide that is secreted extracellularly). All cell lines were positive for p24, as assessed using a p24 ELISA assay kit (Coulter, Fullerton, Calif.). The cell lines also make non-infectious particles that can be rescued into infectious particles by co-expression of an HIV envelope protein.

The HIVgpt cell lines are used as a model system with which to down-regulate HIV expression via PTGS using the methods of this invention. The following example details only one embodiment of the many possible embodiments of the invention, the large RNA hairpin with mismatched regions, for downregulating HIV gag expression.

To generate a reagent of the present invention, six oligonucleotide pairs are generated. The sequences of the oligonucleotides are detailed below. In addition, the coordinates of the HIV gag derived sequences are given. The coordinates and sequences are derived from Genbank Accession number K03455. This is only an example, and other HIV derived sequences besides the ones detailed below are predicted to work as efficiently.

| | |
|---|---|
| Oligonucleotide 1, top strand: | 5′TATTAAGCGGGGGAGAATTTTTTTTT3′ |
| Oligonucleotide 1, bottom strand: | 5′AAAAAAAAATTCTCCCCCGCTTAATA3′ |
| Oligonucleotide 2, top strand: | 5′CAGGTCAGCCAAAATTACCTTTTTTT3′ |
| Oligonucleotide 2, bottom strand: | 5′AAAAAAAGGTAATTTTGGCTGACCTG3′ |
| Oligonucleotide 3, top strand: | 5′GAAAGATTGTTAAGTGTTTTTTTTTT3′ |
| Oligonucleotide 3, bottom strand: | 5′AAAAAAAAAACTCTTAACAATCTTTC3′ |
| Oligonucleotide 4, top strand: | 5′AATTCTCCCCCGCTTAATAGGGGGGG3′ |
| Oligonucleotide 4, bottom strand: | 5′CCCCCCCTATTAAGCGGGGGAGAATT3′ |
| Oligonucleotide 5 ,top strand: | 5′GGTAATTTTGGCTGACCTGGGGGGGG3′ |
| Oligonucleotide 5, bottom strand: | 5′CCCCCCCCAGGTCAGCCAAAATTACC3′ |
| Oligonucleotide 6, top strand: | 5′AATTCTCCCCCGCTTAATAGGGGGGG3′ |
| Oligonucleotide 6, bottom strand: | 5′CCCCCCCTATTAAGCGGGGGAGAATT3′ |

In the above oligonucleotides, the underlined sequences represent Box B sequences (included to create mismatched regions) as defined in more detail elsewhere in the patent. In each oligonucleotide, the remainder of the sequence is derived from HIV (HXB2) gag sequences. The sequences map to the following coordinates: Oligos 1 and 6 map to coordinates 809-827, oligos 2 and 5 map to coordinates 1168-1186, and oligos 3 and 4 map to coordinates 1949-1967.

Following annealing of the top strand of each oligo with its partner bottom strand the annealed oligos are directionally ligated such that the ligation product has the following sequence (only the top strand of the ligation product is shown).

5′TATTAAGCGGGGGAGAATTTTTTTTTCAGGTCAGCCAAAATTACCTTT
TTTTGAAAGATTGTTAAGTGTTTTTTTTTTGGTAATTTTGGCTGACCTGG
GGGGGGAATTCTCCCCCGCTTAATAGGGGGGG3′

The ligation product is PCR amplified using standard techniques and the amplification product is cloned into a vector containing a promoter such as the T7 promoter. An example of such a vector is pCR-Blunt, available from Invitrogen. The amplification product does not need to be directionally ligated into the vector.

Selected Cos7 and RD cells that were stably transfected with the HIVgpt plasmid are transfected with the HIV plasmid encoding the RNA hairpin with regions of mismatch. These cells are co-transfected with a T7 RNA polymerase expression plasmid. The expression plasmid is made by cloning the T7 RNA polymerase gene (GenBank Accession number V01146) into a mammalian expression vector such as pcDNA3 from Invitrogen. Transfection is mediated through Lipofectamine (Gibco-BRL) according to the manufacturer's directions. There also is a control group of cells receiving no RNA and a control group receiving a construct expressing an irrelevant RNA hairpin with mismatched regions (i.e., no HIV sequences). The cells are monitored for p24 synthesis over the course of several weeks. The cells are assayed both by measuring p24 in the media of cells (using the p24 ELISA kit from Coulter, according to the manufacturer's instructions). The construct expressing the HIV sequence-derived RNA hairpin with regions of mismatch is expected to significantly repress HIV p24 synthesis. None of the control cells specifically shut down p24 synthesis.

### Example 6

### Construction of Multi-Hairpin Long dsRNA Vector

The following example describes the construction and testing of a multi-short hairpin long double-stranded RNA vector (udderly structured RNA vector) for the use of eliciting RNA inhibition (RNAi) in cell culture systems and in vivo. The use of this vector allows for the inhibition of a single gene using multiple target sites or the inhibition of multiple genes using single targets for each gene, or for various applications of the “multiple-epitope” approach discussed elsewhere herein

The example described here is used for the inhibition of the gene for the mouse interleukin-12 (IL-12) p40 subunit. The portion of the vector containing the hairpin RNAs corresponding to the mouse IL-12 p40 gene is constructed through the ligation of DNA segments that have the relevant DNA sequences. These sequences correspond to siRNAs that have been shown to be effective in decreasing IL-12 p40 levels in cell culture.

Each of the encoded three short hairpins used are separated from each other by a five nucleotide inter-hairpin sequence. In addition, the 5′-terminal hairpin is preceded by a five nucleotide non-IL-12 sequence and the 3′-terminal hairpin is followed by a five nucleotide non-IL-12 sequence (see below). The sense and antisense portions of each of the IL-12 sequences are separated from each other by a seven-nucleotide loop. The three sets of IL-12 sequences used in hairpin form in the final construct span nucleotides 908-929, 947-968, and 980-1001 of the mouse IL-12 p40 gene (GenBank accession number M86671).

The final 172-nucleotide, IL-12 sequence contains (at the 5′ and 3′ ends of the molecule) a five nucleotide overhang which facilitates cloning of the sequence into a plasmid vector. The three separate IL-12 sequences are ligated together directionally through the use of three sets of annealed oligonucleotides (see below).

The three sets of oligonucleotides used for ligation are:

A1:
5′-tcgacGGTGCGTTCCTCGTAGAGAAGAtcaagagTCTTCTCTACGAG
GAACGCACCgtg-3′
A2:
5′-TGCAcacacGGTGCGTTCCTCGTAGAGAAGActcttgaTCTTCTCTA
CGAGGAACGCACCg-3′
B1:
5′-tgTGCAAAGGCGGGAATGTCTGCGtcaagagCGCAGACATTCCCGCC
TTTGCAgtgtgGA-3′
B2:
5′-TAGCGATCcacacTGCAAAGGCGGGAATGTCTGCGctcttgaCGCAG
ACATTCCCGCCTT-3′
C1:
5′-TCGCTATTACAATTCCTCATtcaagagATGAGGAATTGTAATAGCGA
TCt-3′
C2:
5′-ctagaGATCGCTATTACAATTCCTCATctcttgaATGAGGAATTGTA
A-3′

Three oligo sets are shown as A, B, and C. The number 1 following A, B, or C designates the top strand of the oligo while the number 2 designates the bottom strand. Upper case letters refer to sequences corresponding to the mouse IL-12 p40 gene, lower case letters refer to the inter-hairpin spacer sequences; lower-case bold sequences refer to sequences within the hairpins that form the unpaired loop region.

Oligonucleotide A1 is annealed to oligonucleotide A2; oligonucleotide B1 is annealed to oligonucleotide B2; oligonucleotide C1 is annealed to oligonucleotide C2. The annealed oligonucleotides (which contain overhangs allowing them to anneal to the next set of annealed oligonucleotides) are ligated together such that the following sequence is constructed:

5′-A1/A2-B1/B2-C1/C2-3′
The sequence of the top strand of the ligation
product is:
5′tcgacGGTGCGTTCCTCGTAGAGAAGAtcaagagTCTTCTCTACGAGG
AACGCACCgtgtgTGCAAAGGCGGGAATGTCTGCGtcaagagCGCAGACA
TTCCCGCCTTTGCAgtgtgGATCGCTATTACAATTCCTCATtcaagagAT
GAGGAATTGTAATAGCGATCt3′

The ligation product is cloned into an expression vector containing one promoter (in this case, the HCMV promoter) to drive transcription of the ligation product. It does not matter which strand is transcribed. The vector is designed to contain SalI and XbaI in the polylinker (multiple cloning site). The vector is digested with SalI and XbaI to enable ligation of the original ligation product using the corresponding overhangs built onto the 5′ and 3′ ends of the ligated oligonucleotides.

The plasmid vector, now containing the ligated oligonucleotides, is transfected into cell culture along with a plasmid that expresses mouse IL-12 p40 gene for determination of the inhibitory effect of the RNAi molecules. Transfection is carried out using Lipofectamine (Gibco-BRL) according to the manufacturer's directions.

Media is collected from the supernatant of cells transfected, as described above and also from control cells transfected with only the IL-12 expression vector and cells transfected with an irrelevant udderly structured RNA encoding construct (such as one comprised of HIV sequences) and the murine Il-12 expression vector. Expression of murine IL12 p40 is measured using the Quantikine M-IL-12 p40 Elisa Assay. Only those cells transfected with the Il-12 multihairpin RNA-encoding vector are expected to exhibit significant down-regulation of Il-12 expression. No significant down-regulation of Il-12 (<10% down-regulation) is observed in the other cells.

The IL-12 expression vector can also be administered in vivo to down-regulate IL-12 expression. One example of this is as follows:

Balb/c mice (10 mice/group) are injected intramuscularly, hydrodynamically or intraperitoneally using between 500 ug and 1 mg DNA per injection. DNA is at a concentration of 2 mg/ml and is formulated in 0.5% w/v bupivacaine HCl for injection (Astra Pharmaceutical, Westboro, Mass., among others). All DNA except for DNA to be administered by hydrodynamic delivery is formulated in injection solution (30 mM Na citrate buffer, 150 mM NaCl, 0.1% EDTA [pH 7.6-7.8]. For intramuscular injection, the injection solution containing DNA is adjusted to 0.25%. For intravenous injection the DNA is injected using injection solution containing 0.05% bupivacaine. For hydrodynamic delivery see Human Gene Therapy, 10: 1735-1737 (1999). High Levels of Foreign Gene Expression in Hepatocytes after Tail Vein Injections of Naked Plasmid DNA. Zhang, G. et al. DNA is the vector encoding the IL-12 multihairpin RNA or the irrelevant HIV multi-hairpin RNA. There are also control mice receiving no injection. For intramuscular injection, the dose is divided equally for each quadriceps, with each quadriceps being injected at multiple sites. Sera is collected from mice by retroorbital bleed, every four days for a period of four weeks and assayed for Il-12 p40 levels as described above.

Mice receiving the IL-12-specific multihairpin construct demonstrate a significant reduction in the expression of endogenous IL-12 (e.g., more than 50%, 75%, 90%, or 95% reduction in IL-12 expression), while control mice demonstrate no significant reduction in IL-12 expression (e.g., less than a 20% reduction in IL-12 expression).

### Example 7

### Design and Use of a Vector Designed to Generate Multiple-Short RNA Hairpins

This method enables the expression of multiple short RNA hairpins from a vector. Expression of RNA from this construct results in inhibition of target gene expression by RNA interference. This example describes the construction of a vector that generates RNA with multiple hairpins (MHP) structures, in tandem but separated from each other by several nucleotides. Processing of the RNA generates multiple individual short dsRNA duplexes. This example details downregulation of the gag gene of the HIV, but other constructs based on this strategy for any other gene are predicted to work similarly. This construct is predicted to inhibit the gag gene in cultured mammalian cells and in vivo in animals.

For this example, a vector with a T7 promoter is used for cloning the DNA encoding the multi-short hairpin RNA. A polylinker site is inserted in a unique XhoI/PmeI site present in just downstream of the promoter. The polylinker has the following sequence and unique restriction sites.

```
XhoI PaCI EagI XbaI KpnI EcoRI NheI
TCGAG AAAA TTAATTAA AAAA CGGCCG AAAA TCTAGA AAAA GGTACC AAAA GAATTC AAAA GCTAGC
        C TTTT AATTAATT TTTT GCCGGC TTTT AGATCT TTTT CCATGG TTTT CTTAAG TTTT CGATCG
              NotI PvuI SalI PmeI
AAAA GCGGCCGC AAAA CGATCG AAAA GTCGAC AAAA GTTT
TTTT CGCCGGCG TTTT GCTAGC TTTT CAGCTG TTTT CAAA
```

Five oligonucleotide pairs were designed corresponding to a sequence from the HIV gag gene. Each set encodes a 48 nt hairpin. The hairpin loop in the middle is encoded by six guanosine residues. Each pair when annealed has a 5′ and a 3′ restriction sticky end as indicated below. For example, pair one has a 5′ sticky end corresponding to XhoI site and a 3′ sticky end for a PacI site. The annealed pairs are cloned into the vector cut at the corresponding restriction site pairs, such as XhoI and PacI for oligo pair one, as in the example described above. The source of the sequences is the Gag gene, Genbank accession number K03455.

Oligonucleotide 1, top strand (811-831): XhoI-PacI

5′ TCGAG TTAAGCGGGGGAGAATTAGAT GGGGGG
ATCTAATTCTCCCCCGCTTAA TTAAT 3′

Oligonucleotide 1, bottom strand:

5′ TAA TTAAGCGGGGGAGAATTAGAT CCCCCC
ATCTAATTCTCCCCCGCTTAA C 3′

Oligonucleotide 2, top strand (1168-1188) For EagI-XbaI:

5′ GGCCG CAGGTCAGCCAAAATTACCCT GGGGGG
AGGGTAATTTTGGCTGACCTG T 3′

Oligonucleotide 2, bottom strand:

5′ CTAGA CAGGTCAGCCAAAATTACCCT CCCCCC
AGGGTAATTTTGGCTGACCTG C 3′

Oligonucleotide 3, top strand (1301-1321) For KpnI-EcoRI:

5′ C TGTTTTCAGCATTATCAGAAG GGGGGG
CTTCTGATAATGCTGAAAACA G 3′

Oligonucleotide 3, bottom strand:

5′ AATTC TGTTTTCAGCATTATCAGAAG CCCCCC
CTTCTGATAATGCTGAAAACA GGTAC 3′

Oligonucleotide 4, top strand (1601-1321) For NheI-NotI:

5′ CTAGC ATAAAATAGTAAGAATGTATA GGGGGG
TATACATTCTTACTATTTTAT GC 3′

Oligonucleotide 4, bottom strand:

5′ GGCCGC ATAAAATAGTAAGAATGTATA CCCCCC
TATACATTCTTACTATTTTAT G 3′

Oligonucleotide 5, top strand (1949-1969) For PvuI-SalI:

5′ CG GAAAGATTGTTAAGTGTTTCA GGGGGG
TGAAACACTTAACAATCTTTC G 3′

Oligonucleotide 5, bottom strand:

5′ TCGAC GAAAGATTGTTAAGTGTTTCA CCCCCC
TGAAACACTTAACAATCTTTC CGAT 3′

For illustration, when top and bottom strands of the oligonucletide 1 set are annealed, they have the following sequences.

5′ TCGAG TTAAGCGGGGGAGAATTAGAT GGGGGG ATCTAATTCTCCCCCGCTTAA TTAAT 3′
3′ C AATTCGCCCCCTCTTAATCTA CCCCCC TAGATTAAGAGGGGGCGAATT AAT 5′

The vector cut with XhoI and PacI is annealed to this fragment and then ligated. Similarly, other annealed oligos are sequentially ligated to the growing construct.

During the course of HIV infection, the viral genome is reverse transcribed into a DNA template that is integrated into the host chromosome of infected dividing cells. The integrated copy is now a blueprint from which more HIV particles are made. If the function of a polynucleotide sequence essential to replication and/or pathogenesis of HIV is reduced or inhibited, the viral infection can be treated. This example demonstrates the performance of one embodiment of the method of this invention.

Several cell lines that contain integrated copies of a defective HIV genome, HIVgpt (strain HXB2), have been created. The HIVgpt genome contains a deletion of the HIV envelope gene; all other HIV proteins are encoded. The HIVgpt genome encodes a mycophenolic acid (MPA) resistance gene in place of the envelope gene and thereby confers resistance to MPA. Cells resistant to MPA were clonally amplified. The plasmid used to create these cell lines, HIVgpt, was obtained from the AIDS Research and Reference Reagent Program Catalog. Stably integrated cell lines were made with human rhabdomyosarcoma (RD) and Cos7 cell lines. The lines were made by transfecting cells with the HIVgpt plasmid followed by selection of cells in mycophenolic acid. Cells resistant to MPA were clonally amplified using standard procedures. The media from the cultured clonally expanded cells was assayed for the presence of p24 (an HIV gag polypeptide that is secreted extracellularly). All cell lines were positive for p24, as assessed using a p24 ELISA assay kit (Coulter, Fullerton, Calif.). The cell lines also make non-infectious particles that can be rescued into infectious particles by co-expression of an HIV envelope protein.

The HIVgpt cell lines are used as a model system with which to down-regulate HIV expression via PTGS using the methods of this invention. The following example details only one embodiment, RNA encoding multiple short RNA hairpins, for downregulating HIV gag expression.

Selected Cos7 and RD cells that are stably transfected with the HIVgpt plasmid are transfected with the HIV plasmid encoding the RNA comprised of multiple short RNA hairpin structures. These cells are co-transfected with a T7 RNA polymerase expression plasmid. The expression plasmid is made by cloning the T7 RNA polymerase gene (GenBank Accession number V01146) into a mammalian expression vector such as pcDNA3 from Invitrogen. Transfection is mediated through Lipofectamine (Gibco-BRL) according to the manufacturer's directions. There also is a control group of cells receiving no RNA and a control group receiving a construct expressing an irrelevant RNA with multiple short RNA hairpin structures (i.e., no HIV sequences such as the IL-12 construct described above). The cells are monitored for p24 synthesis over the course of several weeks. The cells are assayed both by measuring p24 in the media of cells (using the p24 ELISA kit from Coulter, according to the manufacturer's instructions). The construct expressing the HIV sequence derived RNA with multiple hairpin structures is expected to significantly repress HIV p24 synthesis. None of the control cells are expected to specifically shut down p24 synthesis.

### Example 8

### Exemplary Constructs that Enable the Efficient Formation of Hairpin dsRNA In Vivo or In Vitro

Constructs encoding a unimolecular hairpin dsRNA may be more desirable for some applications than constructs encoding duplex dsRNA (i.e., dsRNA composed of one RNA molecule with a sense region and a separate RNA molecule with an antisense region) because the single-stranded RNA with inverted repeat sequences more efficiently forms a dsRNA hairpin structure. This greater efficiency is due in part to the occurrence of transcriptional interference arising in vectors containing converging promoters that generate duplex dsRNA. Transcriptional interference results in the incomplete synthesis of each RNA strand thereby reducing the number of complete sense and antisense strands that can base-pair with each other and form duplexes. Transcriptional interference can be overcome, if desired, through the use of (i) a two vector system in which one vector encodes the sense RNA and the second vector encodes the antisense RNA, (ii) a bicistronic vector in which the individual strands are encoded by the same plasmid but through the use of separate cistrons, or (iii) a single promoter vector that encodes a hairpin dsRNA, i.e., an RNA in which the sense and antisense sequences are encoded within the same RNA molecule. Hairpin-expressing vectors have some advantages relative to the duplex vectors. For example, in vectors that encode a duplex RNA, the RNA strands need to find and base-pair with their complementary counterparts soon after transcription. If this hybridization does not happen, the individual RNA strands diffuse away from the transcription template and the local concentration of sense strands with respect to antisense strands is decreased. This effect is greater for RNA that is transcribed intracellularly compared to RNA transcribed in vitro due to the lower levels of template per cell. Moreover, RNA folds by nearest neighbor rules, resulting in RNA molecules that are folded co-transcriptionally (i.e., folded as they are transcribed). Some percentage of completed RNA transcripts is therefore unavailable for base-pairing with a complementary second RNA because of intra-molecular base-pairing in these molecules. The percentage of such unavailable molecules increases with time following their transcription. These molecules may never form a duplex because they are already in a stably folded structure. In a hairpin RNA, an RNA sequence is always in close physical proximity to its complementary RNA. Since RNA structure is not static, as the RNA transiently unfolds, its complementary sequence is immediately available and can participate in base-pairing because it is so close. Once formed, the hairpin structure is predicted to be more stable than the original non-hairpin structure. It will be recognized that in certain embodiments the dsRNA hairpin constructs described herein, e.g., series of multiple hairpin regions, may be “forced” hairpin constructs and/or “partial” hairpin constructs as described in more detail in U.S. Provisional Application 60/399,998, filed 31, Jul. 2002, “Double-stranded RNA Structures and Constructs and Methods for Generating and Using the Same”, C. Satishchandran, Catherine Pachuk, David Shuey, Maninder Chopra, and PCT/US03 . . . , filed 31 Jul. 2003, the teaching of which is incorporated by reference. E.g., regions to “force” hairpin formation may advantageously be added 5′ and 3′ to the desired stem-forming sequences, and/or, in some cases, partial hairpins may be formed and extended by providing an RNA-dependent RNA polymerase.

### Example 9

### Constructs Designed for Improved Expression of siRNAs and shRNAs

Addition of 5′ and/or 3′ Flanking Regions to Counteract Heterogeneous Transcripts Due to Staggered Initiation and Termination:

Promoters vary greatly in their strength (initiation rate) and size (RNA pol II complete promoters may be as large as >1 Kb long while a minimal promoter may be 100 basepairs (bp) long, RNA pol III promoters such as the U6 promoter is about 150 bp long, bacterial promoters are usually about 50 bp long, the bacteriophage T7 promoter is approximately 20 bp long, and a mitochondrial complete promoter is usually about 150 bp long, and a minimal mitochondrial promoter is about 20 bp long).

RNA polymerases inherently initiate transcription preferentially at the first “G” residue downstream from the promoter. Polymerases will also initiate, albeit weakly, from purine residues present at various other positions in a stretch of about 10 basepairs downstream of the promoter, with a preference to initiate at a “G” residue rather than an “A” residue. Due to the variability in the initiation site, transcripts are often heterogeneous at the 5′ ends.

Similarly, the 3′ ends of transcripts are also heterogeneous. However, most eukaryotic transcripts are processed at the 3′ ends by specific ribonucleases. RNA pol II transcripts contain no defined termination site. The polyadenylation signal serves to nucleate proteins that result in nucleolytic cleavage of transcripts downstream from the canonical “AAAUAA” polyadenylation signal. This is followed by enzymatic addition of “A” residues in a sequential manner. However, there is no one cleavage site, and the 3′ ends of transcripts are often staggered. While some RNA pol III transcripts are often processed by RNAase III-like enzymes to mature forms, others are terminated along a stretch of several “T” residues. Although, the RNAase III-assisted processing is invariably precise in its endonucleolytic cleavage, the poly T-based termination results in transcripts that are staggered at the 3′ ends. Similarly, mitochondrial, bacterial, and bacteriophage transcripts also have staggered 3′ termini, although termination of transcription from these promoters is sequence, structure and protein dependent. The methods of the invention directed to variability of termination are only concerned with premature termination as termination past the desired termination site is of no consequence for the functionality of the molecules described herein.

The net result of staggered transcription start and termination is that eiRNA molecules (expressed double-stranded RNA molecules) vary considerably in their length, e.g., by as much as about 20 nucleotides, maximally about 10 nts from each end. This presents a problem particularly in the design of expression vectors that transcribe siRNAs and short hairpin siRNAs (shRNAs) (having a double-stranded region about 19 to 30 bp in length). With these short dsRNAs, staggered transcription initiation and termination could easily result in inactivity. It is the goal of an siRNA expression system to transcribe separate antisense and sense RNA molecules of the desired length (e.g., 19 to 30 nts) from a DNA template(s). Hybridization of these complementary transcripts results in a dsRNA molecule of the desired 19 to 30 basepairs in length. Annealing of complementary sense and antisense sequences present in the same RNA molecule can result in the formation of a shRNA (having a double-stranded region about 19-30 bp in length). The methods described herein enable the maximization of the generation of active siRNAs and shRNAs from DNA-based vectors (plasmid and viral). The design features allow the desired siRNA sequence (19 to 30 mer basepaired molecule) to be included within a transcript which is longer than the desired sequence, comprising several additional nucleotides, up to about 10 nts at either end of the transcript. In some aspects, these additional sequences at the 5′ and 3′ ends of the sequence of interest are designed not to participate in significant base pairing with the desired siRNA or sequences (<about 4 bp), and, preferably, to be unable to base pair with or between themselves. Accordingly, these 5′ and/or 3′ ends will exist as single-stranded RNA in the transcript. Following processing, either through endo- and/or exonucleolytic degradation of the single-stranded portions of the molecule, a double-stranded siRNA or shRNA molecule of the desired size results.

In instances when the design includes sequences that flank the desired si or shRNA, i.e., sequences that do not participate in base pairing, cellular RNAases are sufficient in the degradation of these single-stranded portions following annealing of the two complementary strands. In other aspects of the inventions, the 5′ and 3′ regions are designed to include some hybridizable nucleotides so that some basepairing between the 5′ and the 3′ regions occurs, as illustrated, e.g., in FIG. 8E.

The preferred flanking nucleotides for the 5′ flanking region are 1-4 purines (G is preferred to A) followed by a stretch of nucleotides, preferably pyrimidines, with a preference for C's rather than T's. C's are preferred to T's especially with RNA Pol III systems, where 4 T's (U's) act as a terminator. However, a combination of C's and T's can be used for Pol III; CTCTCTCTCT, CTTCTTCCTTC or CCCTCCCTTCCTCTTC etc. This spacer tract or pyrimidine tract may comprise from 1 to 150 pyrimidines, the number depending of the RNA polymerase to be used; e.g., up to 150 nts may be desirable for transcription by RNA Pol II. If any purines are included in this latter region, A's rather than G's should be used. As the preferred transcription start site will be at a G nucleotide and since there is (are) from 1-4 G residues at the beginning of the 5′ flanking sequence, initiation is forced to be at one of these G residues because initiation will not occur within the string of pyrimidines further downstream. Therefore, transcription will initiate in the 5′ flanking region and will necessarily include all of the desired siRNA/shRNA sequences.

The inclusion of such a 5′ flanking region may also be desirable for expressing constructs described elsewhere in this application. For example, constructs designed to express a series of double-stranded regions separated by single-stranded regions may benefit from such a 5′ flanking sequence that ensures the entire first double-stranded region is present in the transcript.

In all of the designs, the 3′ and the 5′ flanking sequences should not base pair with the siRNA/shRNA sequences (i.e., the stem-loop region) appreciably, or with fewer than four contiguous nts able to do so.

For 3′ flanking sequences, sequences that do not significantly base-pair with the siRNA/shRNA sequences are chosen. Sequences that prematurely induce termination of the polymerases intended to transcribe the vector will be avoided. Several design features, e.g., inclusion of 5′ and 3′ flanking sequences to promote initiation of transcription and termination of transcription as desired, are shown in FIG. 8A.

In instances when the 5′ and 3′ flanking end sequences are designed to base pair, e.g., in FIGS. 8B, 8D, and 8E, the dsRNA molecule that results is longer than the intended siRNA or shRNA and a dsRNA processing enzyme such as Dicer will be needed to generate the desired length siRNAs or shRNAs.

A Vector Encoding an HBV-Derived Hairpin RNA and Vectors Encoding Sense and Antisense HBV RNAs that Form a Duplex siRNA

A plasmid is constructed in which one HBV-specific hairpin RNA is placed under the control of the human U6 promoter.

Vector Descriptions:

In Plasmid A, the hairpin contains sequences that map to coordinates 2911-2935 of Genbank accession #s V01460 and J02203 (i.e., the hairpin contains the sense and antisense versions of this sequence, separated by a loop structure of TTCAAAAGA). Transcription of this hairpin sequence is directed by an RNA pol III promoter, the U6 promoter. Description of U6-based vector systems can be found in Lee et al., Expression of small interfering RNAs targeted against HIV-1 rev transcripts in human cells. Nature Biotechnology, 2002, p. 500-505.

This vector is assessed in an HBV replicon model. Cloning is performed using standard techniques. The DNA sequences representing both strands comprising the flanking and the insert sequence is synthesized and cloned downstream from the promoter. Three consecutive G residues are included at the putative start site. The non-hairpin expression vector is prepared by cloning the same sequence (coordinates 2911-2935 of accession #s V01460 and J02203) in separate cistrons on different plasmids, such that the sequence is oriented in sense with respect to the promoter in one plasmid (Plasmid B) and in the opposite antisense orientation in the other plasmid (Plasmid C). In the experiment detailed below the Plasmids B and C are used together to allow formation of dsRNA structures.

To evaluate the addition of 5′ and 3′ flanking sequences to accommodate the stagger in both the transcription start-site and termination by U6 polymerase, variants of Plasmids A, B, and C are constructed. Additional sequences are appended at both the 5′ and 3′ ends such that they are not complementary and the transcripts are predicted to contain ends that are single-stranded. The 5′ flanking sequence is GGGTTCTCTTC. The G's at the 5′ end serve as initiation sites. The 5′ flanking sequence is followed by the HBV sequences (co-ordinates 2911-2935) as in Plasmid B, the antisense sequence to the same HBV sequence as in Plasmid C, and in the hairpin format with the loop sequence as described above in Plasmid A. All of these plasmids also include additional 3′ flanking sequences that are not capable of hybridizing to any of the 5′ sequences. The sequence CATGTCCATTTT is used at the 3′ end flanking the HBV sequence, where the sequence TTTT serves as the terminator sequence for RNA pol III. These plasmids with sequences flanking the HBV sequence are named Plasmid A-1, B-1 and C-1. The predicted secondary structures are depicted in FIGS. 8A, and 8C (structures I, II, III, and IV are predicted due to stagger or variation in start site and termination site), when Plasmid A-1 is transcribed or when Plasmids B-1 and C-1 are co-transcribed. (Alternate constructs may also be prepared in which the flanking sequence is present only at the 5′ end or at the 3′ end). The transcripts derived from these constructs are predicted to be processed by cellular RNAases that digest single-stranded RNAs, to yield the desired siRNA.

Yet another set of plasmids (Plasmids A2, B2 and C2) similar to Plasmid A-1, B-1 and C-1 is constructed in which the sequences flanking the HBV sequences are designed such that flanking sequences at the 5′ and 3′ ends hybridize with each other to form a longer dsRNA molecule that contains the HBV sequence. These dsRNA constructs are predicted to be processed through a dsRNA cleavage by Dicer (in systems having adequate levels of Dicer enzyme) to result in the siRNA that silences HBV. (FIGS. 8B & 8D). The plasmids prepared in this set are named Plasmids A-2, B-2 and C-2. Plasmid A-2 encodes the hairpin construct, B-2 encodes the sense strand, and C-2 encodes the antisense RNA strand. Plasmids B-2 and C-2 are used together to generate both sense and the antisense RNA strands which will hybridize to result in a dsRNA structure. The HBV sequence is flanked with the following sequences; for Plasmid B-2 at the 5′ end GGGCTCCTCTT (Flank 1S), where the G's at the 5′-most end act as initiation sites and at the 3′ end GGTGTGGTCCCTTTT (Flank 2A), where TTTT is the terminator. For Plasmid C-2, at the 5′ end GGGACCACACC (Flank 2S) where the 5‘most G’s serve as initiation sites, and at the 3′ end, AAGAGGAGCCCTTTT (Flank 1A), in which the terminal TTTT serves as the terminator. Flanks 1S and 1A are designed to hybridize to each other and flanks 2A and 2S are designed to hybridize.

The effector RNA constructs are assessed in an HBV replicon model. For this experiment, the Plasmids A, B and C are compared with Plasmids A-1, B-1, C-1, A-2, B-2 and C-2.

HBV Replicon Model: Silencing HBV Replication and Expression in a Replication Competent Cell Culture Model.

Brief Description of Cell Culture Model

A human liver derived cell line such as the Huh7 cell line is transfected with an infectious molecular clone of HBV, consisting of a terminally redundant viral genome that is capable of transcribing all of the viral RNAs and producing infectious virus. [Yang, P. L., et al., Hydrodynamic injection of viral DNA: a mouse model of acute hepatitis B virus infection. Proc Natl Acad. Sci. USA, 2002. 99(21): p. 13825-30; Guidotti, L. G., et al., Viral clearance without destruction of infected cells during acute HBV infection. Science, 1999. 284(5415): p. 825-9; Thimme, R., et al., CD8(+) T cells mediate viral clearance and disease pathogenesis during acute hepatitis B virus infection. J Virol, 2003. 77(1): p. 68-76.] The replicon used in these studies is derived from the virus sequence found in Gen Bank Accession #s V01460 and J02203. Following internalization into hepatocytes and nuclear localization, transcription of the infectious HBV plasmid from several viral promoters has been shown to initiate a cascade of events that mirrors HBV replication. These events include translation of transcribed viral mRNAs, packaging of transcribed pregenomic RNA into core particles, reverse transcription of pregenomic RNA, and assembly and secretion of virions and HBVsAg particles into the media of transfected cells. This transfection model reproduces most aspects of HBV replication within infected liver cells and is therefore a good cell culture model with which to look at silencing of HBV expression and replication. In this model, cells are co-transfected with the infectious molecular clone of HBV and the effector RNA constructs to be evaluated. The cells are then monitored for loss of HBV expression and replication as described below.

Experimental Procedure: Transfection

Huh7 cells (1×10⁶) are seeded into six-well plates such that they are between 80-90% confluency at the time of transfection. All transfections are performed using Lipofectamine™ (Invitrogen) according to the manufacturer's directions. In this experiment, cells are transfected with 50 ng of the infectious HBV plasmid, and 1.5 ug of the experimental plasmid. Control cells are transfected with 50 ng of the HBV plasmid. An inert filler DNA, pGL3-basic (Promega, Madison Wis.), is added to all transfections to bring total DNA/transfection up to 2.5 ug DNA and are mixed with 20 uL of Lipofectamine. The hairpin effector plasmids (A series) when used singly would result in transcripts capable of forming dsRNA structures, while the B and the C series are used together (transfections are mixed, with 750 ng of each plasmid, such as B with C, B-1 with C-1 and B-2 with C-2.)

Monitoring Cells for Loss of HBV Expression

Following transfection, cells are monitored for the loss or reduction in HBV expression and replication by measuring HBsAg secretion and DNA-containing viral particle secretion. Cells are monitored by assaying the media of transfected cells beginning at 2 days post dsRNA administration and every other day thereafter for a period of three weeks. The Auszyme ELISA, commercially available from Abbott Labs (Abbott Park, Ill.), is used to detect surface Ag (sAg). sAg is measured since surface Ag is associated not only with viral replication but also with RNA polymerase II initiated transcription of the surface Ag cistron in the transfected infectious HBV clone. Since surface Ag synthesis can continue in the absence of HBV replication it is important to down-regulate not only viral replication but also replication-independent synthesis of sAg. Secretion of virion particles containing encapsidated HBV genomic DNA is also measured. Loss of virion particles containing encapsidated DNA is indicative of a loss of HBV replication. Analysis of virion secretion involves a technique that discriminates between naked, immature core particles and enveloped infectious HBV virions [See Thimme, above]. Briefly, pelleted viral particles from the media of cultured cells are subjected to Proteinase K digestion to degrade the core proteins. Following inactivation of Proteinase K, the sample is incubated with RQ1 DNase (Promega, Madison, Wis.) to degrade the DNA liberated from core particles. The sample is digested again with Proteinase K in the presence of SDS to inactivate the DNase as well as to disrupt and degrade the infectious enveloped virion particle. DNA is then purified by phenol/chloroform extraction and ethanol precipitated. HBV specific DNA is detected by gel electrophoresis followed by Southern Blot analysis.

Results

Following transfection of the RNA expression constructs, the cells transfected with the HBV plasmid and experimental Plasmid A-1 demonstrate a greater than 95% decrease in both sAg and viral particle secretion in the media of cells. All of the plasmids are anticipated to be effective to varying degrees when compared with cells transfected with only the HBV plasmid and filler DNA. While, Plasmid A-1 is expected to be the most effective at >95% inhibition, and B+C the least effective at 70% inhibition, others are intermediate in the extent of inhibition of HBV, with A-1>A-2>A>B-1+C-1>B-2+C-2>B+C.

Accordingly, the 5′ flanking region, comprising an initiator sequence and an optional spacer region, i.e., “5′ initiator/spacer”, and/or the 3′ flanking region, comprising a spacer and a terminator, “3′ spacer/terminator” can advantageously be used to ensure transcription of the entire desired transcript sequences. This is particularly important in systems designed to express siRNAs and shRNAs, which have a sequence of 19-30 basepairs, preferably 19-27, more preferably 19-24, even more preferably 21-23 basepairs in double-stranded conformation. The inclusion of such a 5′ flanking region may also be desirable for expressing constructs described elsewhere in this application. For example, constructs designed to express a series of double-stranded regions separated by single-stranded regions may benefit from such a 5′ flanking sequence that ensures the entire first double-stranded region is present in the transcript. This is particularly advantageous where the first double-stranded region to be expressed is a single RNA strand with sense and antisense sequences designed to assume a stem loop or hairpin conformation, and especially where double-stranded region includes a single “sequitope” of between 19 and 30 nucleotides have substantial sequence identity with a target polynucleotide, and especially where the double-stranded region is a sequence of between 19 and 30 nts.

Several other plasmid constructs were also evaluated for their abilities to silence HBV expression n the replicon model. These plasmids were similar to but variants of the plasmid A-2, designed to encode various RNA structures through hybridization of the flanking regions, but comprising the dsRNA structures containing HBV specific shRNA sequences (FIG. 8E). These plasmids were as effective as plasmid A-1 in silencing HBV replicons.

### Example 10

### Construction of dsRNA Expression Constructs with Stabilizing 5′ Hairpin and Linker Region

In particularly desirable embodiments of the invention, the dsRNA expression construct will include a stabilizing 5′ hairpin-linker region as described in the following example. The hairpin is termed a “BM” hairpin or “Bernie Moss” hairpin, and is described in Fuerst T R and Moss B (1989) “Structure and stability of mRNA synthesized by vaccinia virus-encoded bacteriophage T7 RNA Polymerase in mammalian cells.” J. Mol. Biol. 206:333-348. Such a 5′ hairpin-linker region stabilizes the proximate transcript region and protects the 5′ terminus of the transcript from degradation, and/or loss due to staggered initiation of transcription. In some embodiments, the 5′ hairpin-linker region may be advantageously used in conjunction with a 5′ flanking region as described in Example 9, e.g., dsRNA expression construct may be engineered to include a 5′ flanker to “force” transcription initiation as desired, followed by a “stabilizing” 5′ hairpin-linker region as described in this example, followed by one or more “effector” hairpins targeted to one or more polynucleotide sequences of interest to be silenced.

An exemplary method of making such a dsRNA expression construct including a hairpin-linker sequence preceding a dsRNA hairpin of the invention is as follows. The sequence of the linker region was designed to lack homology to known human genome sequences. Any similar sequence could be used. Such a stabilizing hairpin region, or stabilizing hairpin-linker region, could desirably be employed with any expressed dsRNA structure, including single hairpin dsRNAs, multiple dsRNA hairpin constructs, multiple dsRNA regions separated by mismatched regions, and partial and/or forced hairpins, as described elsewhere herein. For simplicity, the following example describes a construct encoding a protective/stabilizing 5′ hairpin-linker region preceding what is termed the “Effector Hairpin”, a single short dsRNA hairpin (shRNA) having sequence identity to a target polynucleotide. It will be understood that any dsRNA effector region could advantageously be stabilized in this way, including any multiple dsRNA hairpin, multiple dsRNA regions separated by mismatched regions, partial and/or forced hairpins, etc., and other dsRNA structures known to those of skill in the art.

Two primers were designed as follows:

### Forward Primer:

5′CGCGCCTAATACGACTCACTATAGGGAGACCACAACGGTTTCCCTCTA
GCGGGATCAAAAAAACGCCGCAGACACATCCATTCAAGAGATGGATGTGT
CTGCGGCGTTTTTTATCTGTTTTTC 3′

The reverse primer:

5′CTAGGAAAAACAGATAAAAAACGCCGCAGACACATCCATCTCTTGAAT
GGAT [symbol] TGTCTGCGGCGTTTTTTTGATCCCGCTAGAGGGAAACCGTTGTGG
TCTCCCTATA [symbol] TGAGTCGTATTAGG3′

[symbol]

indicates data missing or illegible when filed

The primers were annealed and the resulting duplex DNA would be as follows:

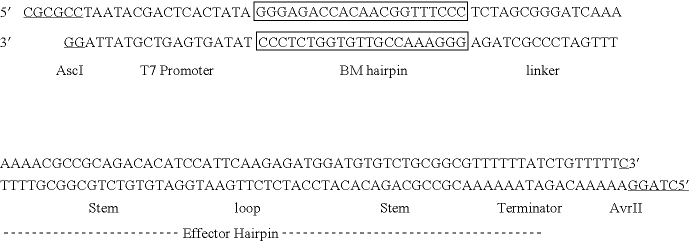

These duplex-forming oligos with the AscI and AvrII restriction sites were cloned into a plasmid vector with the same sites. The plasmid vector already includes a T7 RNA Polymerase gene expressed under the control of a RSV promoter. The resulting construct when introduced into a mammalian cell will express T7 RNA Polymerase, which in turn will produce a transcript from the sequence starting immediately after the T7 Promoter and ending in the T7 terminator. The structure of this RNA transcript allows formation of two hairpins labeled as “BM hairpin” and “Effector Hairpin”, separated by a 15 bp linker. The presence of the BM hairpin and a linker preceding the “Effector hairpin region” prevents its degradation. The structure of an RNA transcript, including the BM hairpin, the linker region, and the Effector dsRNA hairpin above, is shown in FIG. 9.

### Example 11

### Multiple-Epitope Double-Stranded RNA Approach

Significant advantages can be obtained by using dsRNA with segments or epitopes derived from (1) sequences representing multiple genes of a single organism; (2) sequences representing one or more genes from a variety of different organisms; and/or (3) sequences representing different regions of a particular gene. Using this approach, a singular species of dsRNA can be engineered to simultaneously target many different genes and/or many organisms, e.g, pathogens, including viral and/or bacterial pathogenic agents. Alternatively, the singular species of dsRNA can be used to target a subset of genes or organisms on one occasion and the same or a second subset on another occasion. The dsRNA can be, e.g., a duplex or a hairpin and can be encoded by a DNA or RNA vector. The RNA can be expressed intracellularly in the host or made in vitro and then subsequently administered to the host, as described herein. This “multiple epitope,” at least partially double-stranded RNA molecules can assume a variety of structural variations, including the partial hairpins and forced hairpins described in detail herein, and further, as described, for example, in Pachuk and Satishchandran, WO 00/63364, the teaching of which is incorporated herein by reference. The host cell can be a cell in vitro or in vivo, such as a cell in a tissue or an organism (e.g., a cell in a plant or animal, including invertebrate and vertebrate animals, or mammal such as a human or commercially important species such as a bovine, equine, canine, feline, or avian).

One particularly desirable multiple epitope approach involves targeting both a selected target gene(s) and the promoter(s) which drives transcription of that gene, resulting in a combination of post-transcriptional and transcriptional gene silencing (PTGS and TGS). This combination of gene silencing has the advantage of achieving a rapid gene silencing response that is maintained for a long duration or permanently in the host.

Advantages of a Multiple Epitope Double-Stranded RNA Approach

Because a singular species of dsRNA can simultaneously target and silence many genes (e.g., genes from multiple pathogens or genes associated with multiple diseases), a multiple epitope dsRNA can be used for many different indications in the same subject or used for a subset of indications in one subject and another subset of indications in another subject. Due to the growing concern about terrorism and the potential threat of biological warfare, a multiple epitope dsRNA is useful as a non-toxic agent that can provide protection against a number of different organisms for an extended period of time, if not permanently. Particularly promising is a DNA construct capable of intracellular expression in a host of an at least partially double-stranded RNA comprising dsRNA sequences exhibiting homology with one or more genes of a number of different potential pathogenic organisms, including viruses such as smallpox, Ebola, Marburg, HIV-1, HIV-2, Dengue, Yellow fever, or influenza. The dsRNA can also include sequences for host cellular receptors for viral and/or bacterial genes and/or viral and/or bacterial toxins (e.g., cellular receptors for toxins from Anthrax, Diphtheria, or Botulinum toxin). For such applications, the ability to express long dsRNA molecules (e.g., dsRNA molecules with sequences from multiple genes) without invoking the dsRNA stress response is highly desirable. For example, by using a series of sequences, each, e.g., as short as 19-21 nucleotides, preferably 100 to 600 nucleotides, or easily up to 1, 2, 3, 4, 5, or more kilobases such that the total length of such sequences is within the maximum capacity of the selected plasmid (e.g., 20 kilobases in length), a single such pharmaceutical composition can provide protection against a large number of pathogens and/or toxins at a relatively low cost and low toxicity. Importantly, this same approach can be used to provide protection against biological warfare agents that affect important food crops such as wheat or rice or commercially important animals such as cattle, sheep, goats, pigs, poultry, or fish.

Examples of viral pathogens that may be suitable targets for application of the multiple epitope dsRNA approach include HIV-1, HIV-2, smallpox, vaccinia, encephalitic viruses (e.g., West Nile, Japanese encephalitis, and equine encephalitis), Dengue, Yellow fever, Ebola, Marburg, measles, polio, influenza, hepatitis viruses (e.g., Hepatitis A, B, and C), Herpes simplex 1 and 2, EBV, HCMV, as well as species of the Retrovirus, Herpesvirus, Hepadnavirus, Poxvirus, Parvovirus, Papillomavirus, and Papovavirus families. Some of the more desirable viral infection to treat or prevent with this method include, without limitation, infections caused by HIV, HBV, HSV, CMV, HPV, HTLV, or EBV. Particularly suitable for such treatment are DNA viruses or viruses that have an intermediary DNA stage. The target gene(s) or fragment thereof is desirably a virus polynucleotide sequence that is necessary for replication and/or pathogenesis of the virus in an infected mammalian cell. Among such target polynucleotide sequences are protein-encoding sequences for proteins necessary for the propagation of the virus, e.g., the HIV gag, env, and pol genes as well as necessary regulatory genes; the HPV6 L1 and E2 genes; the HPV 11 L1 and E2 genes; the HPV16 E6 and E7 genes; the HPV18 E6 and E7 genes; the HBV surface antigen, core antigen, and reverse transcriptase; the HSV gD gene; the HSVvp16 gene; the HSVgC, gH, gL, and gB genes; the HSV ICPO, ICP4 and ICP6 genes; Varicalla zoster gB, gC, and gH genes; the BCR-abl chromosomal sequences, and non-coding viral polynucleotide sequences which provide regulatory functions necessary for transfer of the infection from cell to cell, e.g., HIV LTR and other viral promoter sequences, such as HSV vp16 promoter, HSVICP0 promoter; HSV-ICP4, ICP6 and gD promoters, the HSV surface antigen promoter; or the HBV pre-genomic sequence. Other exemplary targets are described in Pachuk and Satishchandran, WO 00/63364, and in U.S. Pat. No. 6,506,559, Fire et al., the teaching of which is hereby incorporated by reference.

The use of multiple epitopes derived from one or more genes from multiple strains and/or variants of a highly variable or rapidly mutating pathogen such as HIV, HCV, or influenza can also be very advantageous. For example, a singular dsRNA species that recognizes and targets multiple strains and/or variants of the influenza virus can be used as a universal treatment or vaccine for the various strains/variants of influenza.

The ability to silence multiple genes of a particular pathogen such as HIV prevents the selection of, in this case, HIV “escape mutants.” In contrast, typical small molecule treatment or vaccine therapy that only targets one gene or protein results in the selection of pathogens that have sustained mutations in the target gene or protein and the pathogen thus becomes resistant to the therapy. By simultaneously targeting a number of genes of the pathogen and/or extensive regions of the pathogen using the multiple epitope approach of the present invention, the emergence of such “escape mutants” is effectively precluded.

This multiple epitope approach is also particularly suitable for the treatment of cancers that result from the over-expression of more than one gene product. Such gene products, by definition, are needed to maintain the cancerous state of the tumor cell or tumor. One singular dsRNA species can act to target the multiple RNA molecules encoding these different gene products or a subset of these gene-products. Thus, one pharmaceutically active dsRNA silences the multiple components that have led to the cancerous phenotype. Examples of human cancers include cervical, ovarian, lung, colon, leukemias, lymphomas, breast, prostate, testicular, uterine, melanoma, liver, head and neck, malignant brain, and stomach cancer. Oncogenes are suitable targets for the dsRNA of the invention (including, e.g., ABL1, BCL1, BCL2, BCL6, CBFA2, CBL, CSF1R, ERBA, ERBB, EBRB2, FGR, FOS, FYN, HCR, HRAS, JUN, KRAS, LCK, LYN, MDM2, MLL, MYB, MYC, MYCL1, MYCN, NRAS, and RAS). Tumor suppressor genes, (e.g., APC, BCRA1, BCRA2, MADH4, MCC, NF1, NF2, RB1, and TP530), enzymes (e.g., kinases), cancer-associated viral targets (e.g., HPV E6/E7 virus-induced cervical carcinoma, HTLV-induced cancer, and EBV-induced cancers such as Burkitt's Lymphoma) can also be targeted. In the latter instance, a composition can be administered in which the target polynucleotide is a coding sequence or fragment thereof, or a non-expressed regulatory sequence for an antigen or sequence that is required for the maintenance of the tumor in the host animal. Exemplary targets include HPV16 E6 and E7 and HPV 18 E6 and E7 sequences. Others may be readily selected by one of skill in the art. In developing multiple epitope constructs directed toward a cancer-related polynucleotide sequence with a single point mutation as compared to the normal sequence, it may be advantageous to string together a series of overlapping 21-mers (19-23 mers), each of which contains the mutation that distinguishes the abnormal sequence.

It will be readily recognized that the dsRNA constructs of the invention, which comprise a series of double-stranded regions separated by single-stranded regions, including the “udderly” structured constructs comprising a series of short hairpins, provide a particularly advantageous embodiment of the multi-epitope approach described herein. Each double-stranded region can provide a particularly effective dsRNA epitope or target region.

Pharmaceutical Compositions

A pharmaceutical composition can be prepared as described herein comprising a DNA plasmid construct expressing, under the control of a bacteriophage T7 promoter, a dsRNA substantially homologous to, e.g., one or more genes from the smallpox virus and human cell receptor sequences for the Anthrax toxin. The T7 RNA polymerase can be co-delivered and expressed from the same or another plasmid under the control of a suitable promoter e.g., hCMV, simian CMV, or SV40. In some embodiments, the same or another construct expresses the target gene (e.g., a target smallpox gene) contemporaneously with the dsRNA homologous to the target smallpox gene. The pharmaceutical composition is prepared in a pharmaceutical vehicle suitable for the particular route of administration. For IM, SC, IV, intradermal, intrathecal or other parenteral routes of administration, a sterile, non-toxic, pyrogen-free aqueous solution such as Sterile Water for Injection, and, optionally, various concentrations of salts, e.g., NaCl, and/or dextrose, (e.g., Sodium Chloride Injection, Ringer's Injection, Dextrose Injection, Dextrose and Sodium Chloride Injection, and Lactated Ringer's Injection) is commonly used. Optionally, other pharmaceutically appropriate additives, preservatives, or buffering agents known to those in the art of pharmaceutics are also used. If provided in a single dose vial for injection, the dose will vary as determined by those of skill in the art of pharmacology, but may typically contain between 5 mcg to 500 mcg of the active construct. If deemed necessary, significantly larger doses may be administered without toxicity, e.g., up to 5-10 mg.

The DNA and/or RNA constructs of the invention may be administered to the host cell/tissue/organism as “naked” DNA, RNA, or DNA/RNA, formulated in a pharmaceutical vehicle without any transfection promoting agent. More efficient delivery may be achieved as known to those of skill in the art of DNA and RNA delivery, using e.g., such polynucleotide transfection facilitating agents known to those of skill in the art of RNA and/or DNA delivery. The following are exemplary agents: cationic amphiphiles including local anesthetics such as bupivacaine, cationic lipids, liposomes or lipidic particles, polycations such as polylysine, branched, three-dimensional polycations such as dendrimers, carbohydrates, detergents, or surfactants, including benzylammonium surfactants such as benzylkonium chloride. Non-exclusive examples of such facilitating agents or co-agents useful in this invention are described in U.S. Pat. Nos. 5,593,972; 5,703,055; 5,739,118; 5,837,533; 5,962,482; 6,127,170; and 6,379,965, as well as U.S. Provisional Application 60/378,191, filed 6 May 2002 and International Patent Application Nos. PCT/US03/14288, filed May 6, 2003 (multifunctional molecular complexes and oil/water cationic amphiphile emulsions), and PCT/US98/22841; the teaching of which is hereby incorporated by reference. U.S. Pat. Nos. 5,824,538; 5,643,771; and 5,877,159 (incorporated herein by reference) teach delivery of a composition other than a polynucleotide composition, e.g., a transfected donor cell or a bacterium containing the dsRNA-encoding compositions of the invention.

### Example 12

### Exemplary Methods for Enhancing dsRNA-Mediated Gene Silencing

Mammalian Origin of Replication

An origin of replication enables the DNA plasmid to be replicated upon nuclear localization and thus enhances gene silencing. The advantage is that more plasmid is available for nuclear transcription and therefore more RNA effector molecules are made (e.g., more hairpins and/or more duplexes). Many origins are species-specific and work in several mammalian species but not in all species. For example, the SV40 T origin of replication (e.g., from plasmid pDsRed1-Mito from Clontech; U.S. Pat. No. 5,624,820) is functional in mice but not in humans. This origin can thus be used for vectors that are used or studied in mice. Other origins that can be used for human applications, such as the EBNA origin (e.g., plasmids pSES.Tk and pSES.B from Qiagen). DNA vectors containing these elements are commercially available, and the DNA segment encoding the origin can be obtained using standard methods by isolating the restriction fragment containing the origin or by PCR amplifying the origin. The restriction maps and sequences of these vectors are available publicly and enable one skilled in the art to amplify these sequences or isolate the appropriate restriction fragment. These vectors replicate in the nuclei of cells that express the appropriate accessory factors such as SV40 TAg and EBNA. The expression of these factors is easily accomplished because some of the commercially available vectors (e.g., pSES.Tk and pSES.B from Qiagen) that contain the corresponding origin of replication also express either SV40 Tag or the EBNA. These DNA molecules containing the origin of replication can be easily cloned into a vector of interest (e.g., a vector expressing a dsRNA such as a hairpin or duplex) by one skilled in the art. These vectors are then co-transfected, injected, or administered with a vector expressing EBNA or Tag to enable replication of the plasmid bearing the EBNA or Tag origin of replication, respectively. Alternatively, the genes encoding EBNA or Tag are cloned into any another expression vector designed to work in the cells, animal, or organism of interest using standard methods. The genes encoding EBNA and Tag can also be cloned into the same vector bearing the origin of replication. Suitable origins of replication are not limited to Tag and EBNA; for example, Replicor in Montreal has identified a 36 base-pair mammalian origin consensus sequence that permits the DNA sequence to which it is attached to replicate (as reviewed in BioWorld Today, Aug. 16, 1999, Volume 10, No. 157). This sequence does not need the co-expression of auxiliary sequences to enable replication.

Replication of dsRNA

Alternatively or additionally, the transcribed dsRNA molecules can be amplified. RNA can be replicated by a variety of RNA-dependent RNA polymerases provided the appropriate replication signals are encoded at the 3′ ends of the RNA molecules. Examples are provided in the following references: Driver et al., Ann NY Acad Sci 1995, 261-264, and Dubensky et al, J Virol, 1996, 508-519. Other exemplary RNA dependent-RNA polymerases (e.g., viral, plant, invertebrate, or vertebrate such as mammalian or human polymerases) are listed in Table 1. Additional suitable RNA dependent-RNA polymerases include alphaviral polymerases, Semliki Forest viral polymerases, and polymerases from mammalian viruses, invertebrates, and plants. The RNA molecules that are replicated by cytoplasmic RNA polymerases can be transcribed in the nucleus followed by cytoplasmic localization, or they can be transcribed in the cytoplasm.

### Example 13

### Exemplary Methods for the Administration of dsRNA

The short dsRNA molecules and/or long dsRNA molecules of the invention may be delivered as “naked” polynucleotides, by injection, electroporation, and any polynucleotide delivery method known to those of skill in the field of RNA and DNA. For example, in vitro synthesized dsRNA may be directly added to a cell culture medium. Uptake of dsRNA is also facilitated by electroporation using those conditions required for DNA uptake by the desired cell type. RNA uptake is also mediated by lipofection using any of a variety of commercially available and proprietary cationic lipids, DEAE-dextran-mediated transfection, microinjection, protoplast fusion, calcium phosphate precipitation, viral or retroviral delivery, local anesthetic RNA complex, or biolistic transformation.

Alternatively, the RNA molecules may by delivered by an agent (e.g., a double-stranded DNA molecule) that generates an at least partially double-stranded molecule in cell culture, in a tissue, or in vivo in a vertebrate or mammal. The DNA molecule provides the nucleotide sequence which is transcribed within the cell to become an at least partially double-stranded RNA. These compositions desirable contain one or more optional polynucleotide delivery agents or co-agents, such as a cationic amphiphile local anesthetic such as bupivacaine, a peptide, cationic lipid, a liposome or lipidic particle, a polycation such as polylysine, a branched, three-dimensional polycation such as a dendrimer, a carbohydrate, a cationic amphiphile, a detergent, a benzylammonium surfactant, one or more multifunctional cationic polyamine-cholesterol agents disclosed in U.S. Pat. No. 5,837,533, and U.S. Pat. No. 5,837,533; U.S. Pat. No. 6,127,170; U.S. Pat. No. 5,962,428, U.S. Pat. No. 6,197,755, WO 96/10038, published Apr. 4, 1996, WO 94/16737, published Aug. 8, 1994, and U.S. Provisional Application 60/383,191, filed 6 May 2002 and PCT/US03/14288, filed May 6, 2003 (multifunctional molecular complexes and oil/water cationic amphiphile emulsions), the teaching of which are hereby incorporated by reference.

For administration of dsRNA as taught in U.S. Ser. No. 60/375,636 filed Apr. 26, 2002 and U.S. Ser. No. 10/425,006 filed Apr. 28, 2003 “Methods for Silencing Genes Without Inducing Toxicity”, C. Pachuk, the teaching of which is incorporated herein by reference, (e.g., a short dsRNA to inhibit toxicity or a short or long dsRNA to silence a gene) to a cell or cell culture, typically between 50 ng and 5 ug, such as between 50 ng and 500 ng or between 500 ng and 5 ug dsRNA is used per one million cells. For administration of a vector encoding dsRNA (e.g., a short dsRNA to inhibit toxicity or a short or long dsRNA to silence a gene) to a cell or cell culture, typically between 10 ng and 2.5 ug, such as between 10 ng and 500 ng or between 500 ng and 2.5 ug dsRNA is used per one million cells. Other doses, such as even higher doses may also be used.

For administration of dsRNA (e.g., a short dsRNA to inhibit toxicity or a short or long dsRNA to silence a gene) to an animal, typically between 10 mg to 100 mg, 1 mg to 10 mg, 500 ug to 1 mg, or 5 ug to 500 ug dsRNA is administered to a 90-100 pound person or animal (in order of increasing preference. For administration of a vector encoding dsRNA (e.g., a short dsRNA to inhibit toxicity or a short or long dsRNA to silence a gene) to an animal, typically between 100 mg to 300 mg, 10 mg to 100 mg, 1 mg to 10 mg, 500 ug to 1 mg, or 50 ug to 500 ug dsRNA is administered to a 90-100 pound person (in order of increasing preference. The dose may be adjusted based on the weight of the animal. In some embodiments, about 1 to 10 mg/kg or about 2 to 2.5 mg/kg is administered. Other doses may also be used.

For administration in an intact animal, typically between 10 ng and 50 ug, between 50 ng and 100 ng, or between 100 ng and 5 ug of dsRNA or DNA encoding a dsRNA is used. In desirable embodiments, approximately 10 ug of a DNA or 5 ug of dsRNA is administered to the animal. With respect to the methods of the invention, it is not intended that the administration of dsRNA or DNA encoding dsRNA to cells or animals be limited to a particular mode of administration, dosage, or frequency of dosing; the present invention contemplates all modes of administration sufficient to provide a dose adequate to inhibit gene expression, prevent a disease, or treat a disease. The doses may be adjusted based on the weight of the animal, the effect to be achieved, and the route of administration, as can be determined without undue experimentation by those of skill in the art of pharmacology.

If desired, short dsRNA is delivered before, during, or after the delivery of dsRNA (e.g., a longer dsRNA) that might otherwise be expected to induce cytotoxicity. Modulation of cell function, gene expression, or polypeptide biological activity may then be assessed in the cells or animals.

### Example 14

### Exemplary Methods for Using the dsRNAs of the Invention in dsRNA-Mediated Gene Silencing to Determine or Validate the Function of a Gene

The dsRNAs of the invention, including the dsRNA partial and/or forced hairpin structures, and the dsRNA expression constructs encoding such partial and/or forced hairpin structures, and kits providing such dsRNAs and/or dsRNA expression constructs, including such kits which provide a source of RdRp, may be advantageously utilized in various functional genomics applications as described in more detail below.

DsRNA-mediated gene silencing can be used as a tool to identify and validate specific unknown genes involved in cell function, gene expression, and polypeptide biological activity. Since novel genes are likely to be identified through such methods, PTGS is developed for use in validation and to identify novel targets for use in therapies for diseases, for example, cancer, neurological disorders, obesity, leukemia, lymphomas, and other disorders of the blood or immune system.

The dsRNAs and dsRNA expression constructs of the invention can be advantageously used in the methods taught in U.S. Published Application 2002/0132257 and European Published Application 1229134, “Use of post-transcriptional gene silencing for identifying nucleic acid sequences that modulate the function of a cell”, the teaching of which is hereby incorporated by reference. The methods involve the use of double-stranded RNA expression libraries, double-stranded RNA molecules, and post-transcriptional gene silencing techniques. Particularly preferred for utilization of the dsRNAs and dsRNA expression constructs of the invention are such methods wherein cDNA libraries are utilized to obtain a single integration per cell and expression of a single dsRNA per cell. In some embodiments, once a stable integrant containing five or fewer, and desirably no episomal expression vectors, transcription is induced, allowing dsRNA to be expressed in the cells. This method ensures that, if desired, only one species or not more than about five species of dsRNA is expressed per cell, as opposed to other methods that express hundreds to thousands of double-stranded species.

These methods provide a highly efficient means for identifying nucleic acid sequences which, e.g., confer or are associated with a detectable phenotype, e.g., nucleic acid sequences that modulate the function of a cell, the expression of a gene in a cell, or the biological activity of a target polypeptide in a cell. A detectable phenotype may include, for example, any outward physical manifestation, such as molecules, macromolecules, structures, metabolism, energy utilization, tissues, organs, reflexes, and behaviors, as well as anything that is part of the detectable structure, function, or behavior of a cell, tissue, or living organism. Such methods are useful in a variety of valuable applications including high throughput screening methods for identifying and assigning functions to unknown nucleic acid sequences, as well as methods for assigning function to known nucleic acid sequences. A particularly advantageous aspect of such methods is that the transformation of vertebrate cells, including mammalian cells, and the formation of double-stranded RNA are carried out under conditions that inhibit or prevent an interferon response or a double-stranded RNA stress response.

The dsRNAs and dsRNA expression constructs of the invention can be advantageously used in the following methods which use site-specific recombination to obtain single integrants (or desirably no more than five) of dsRNA expression cassettes at the same locus of all cells in the target cell line, allowing stable and uniform expression of the dsRNA in all of the integrants. A dsRNA expression library derived from various cell lines is used to create a representative library of stably integrated cells, each cell within the target cell line containing a single integrant. Cre/lox, Lambda-Cro repressor, and Flp recombinase systems or retroviruses may be used to generate these singular integrants of dsRNA expression cassettes in the target cell line. A desirable vector may comprise two convergent T7 promoters, two convergent SP6 promoters, or one convergent T7 promoter and one convergent SP6 promoter, a selectable marker, and/or a loxP site. (Satoh et al., J. Virol. 74:10631-10638, 2000; Trinh et al., J. Immunol. Methods 244:185-193, 2000; Serov et al., An. Acad. Bras. Cienc. 72-0.389-398, 2000; Grez et al, Stem Cells. 16:235-243, 1998; Habu et al., Nucleic Acids Symp. Ser. 2:295-296, 1999; Haren et al., Annu. Rev. Microbiol. 53:245-281, 1999; Baer et al., Biochemistry 39:7041-7049, 2000; Follenzi et al., Nat. Genet. 25:217-222, 2000; Hindmarsh et al., Microbiol. Mol. Biol. Rev. 63:836-843, 1999; Darquet et al., Gene Ther. 6:209-218, 1999; Darquet et al., Gene Ther. 6:209-218, 1999; Yu et al, Gene 223:77-81, 1998; Darquet et al., Gene Ther. 4:1341-1349, 1997; and Koch et al., Gene 249:135-144, 2000). These systems are used singularly to generate singular insertion clones, and also in combination.

The following exemplary sequence specific integrative systems use short target sequences that allow targeted recombination to be achieved using specific proteins: FLP recombinase, bacteriophage Lambda integrase, HIV integrase, and pilin recombinase of Salmonella (Seng et al. Construction of a Flp “exchange cassette” contained vector and gene targeting in mouse ES cell; A book chapter PUBMED entry 11797223-Sheng Wu Gong Cheng Xue Bao. 2001 September; 17(5):566-9; Liu et al., Nat Genet. 2001 Jan. 1; 30(1):66-72; Awatramani et al., Nat Genet. 2001 November; 29(3):257-9; Heidmann and Lehner, Dev Genes Evol. 2001 September; 211(8-9):458-65; Schaft et al., Genesis 2001 September; 31(1): 6-10; Van Duyne, Annu Rev Biophys Biomol Struct. 2001; 30:87-104; Lorbach et al., J Mol Biol. 2000 Mar. 10; 296(5):1175-81; Darquet et al., Gene Ther. 1999 February; 6(2):209-18; Bushman and Miller, J Virol. 1997 January; 71(1):458-64; Fulks et al., J. Bacteriol. 1990 January; 172(1):310-6). A singular integrant is produced by randomly inserting the specific sequence (e.g., loxP in the cre recombinase system) and selecting or identifying the cell that contains a singular integrant that supports maximal expression. For example, integrants that show maximal expression following random integration can be identified through the use of reporter gene sequences associated with the integrated sequence. The cell can be used to specifically insert the expression cassette into the site that contains the target sequence using the specific recombinase, and possibly also remove the expression cassette that was originally placed to identify the maximally expressing chromosomal location.

A skilled artisan can also produce singular integrants using retroviral vectors, which integrate randomly and singularly into the eukaryotic genome. In particular, singular integrants can be produced by inserting retroviral vectors that have been engineered to contain the desired expression cassette into a naive cell and selecting for the chromosomal location that results in maximal expression (Michael et al., EMBO Journal, vol 20: pages 2224-2235, 2001; Reik and Murrell., Nature, vol. 405, page 408-409, 2000; Berger et al., Molecular Cell, vol. 8, pages 263-268). One may also produce a singular integrant by cotransfecting the bacterial RecA protein with or without nuclear localization signal along with sequences that are homologous to the target sequence (e.g., a target endogenous sequence or integrated transgene sequence). Alternatively, a nucleic acid sequence that encodes a RecA protein with nuclear localization signals can be cotransfected (Shibata et al., Proc. Natl. Acad. Sci. U.S.A. 2001 Jul. 17; 98(15):8425-32; Muyrers et al., Trends Biochem. Sci. 2001 May; 26(5):325-31; Paul et al., Mutat. Res. 2001 Jun. 5; 486(1): 11-9; Shcherbakova et al., Mutat. Res. 2000 Feb. 16; 459(1):65-71; Lantsov. Mol. Biol. (Mosk). 1994 May-June; 28(3):485-95). Other methods as taught in U.S. Published Application 2002/0132257 and European Published Application EP1229134, “Use of post-transcriptional gene silencing for identifying nucleic acid sequences that modulate the function of a cell”, are also contemplated as useful applications of the unique dsRNA hairpin constructs and dsRNA expression constructs of the invention.

See also the methods and teaching of published applications WO 00/01846, EP1093526, and EP 1197567, “Characterization of Gene Function Using Double-Stranded RNA Inhibition”, incorporated herein by reference, which provides a method of identifying DNA responsible for conferring a particular phenotype in a cell. The method comprises constructing a cDNA or genomic library of the DNA of a cell in a suitable vector in an orientation relative to a promoter(s) capable of initiating transcription of the cDNA or DNA to double-stranded (ds) RNA upon binding of an appropriate transcription factor to said promoter(s); introducing the library into one or more cells comprising said transcription factor; and identifying and isolating a particular phenotype of the cell comprising the library and identifying the DNA or cDNA fragment from the library responsible for conferring the phenotype.

See also published applications WO 99/32619 and EP1042462, “Genetic Inhibition by Double-Stranded RNA”, which teach methods of identifying gene function in an organism comprising the use of double-stranded RNA to inhibit the activity of a target gene of previously unknown function. High throughput screening methods wherein dsRNAs are produced from gene libraries, e.g., genomic DNA or mRNA (cDNA and eRNA) libraries derived from a target cell or organism.

While less desirable, all of such functional genomics methods may utilize randomized nucleic acid sequences or a given sequence for which the function is not known, as described in, e.g., U.S. Pat. No. 5,639,595, the teaching of which is hereby incorporated by reference.

The dsRNA structures and dsRNA expression constructs of the present invention may be used in methods to identify unknown targets that result in the modulation of a particular phenotype, an alteration of gene expression in a cell, or an alteration in polypeptide biological activity in a cell, using either a library based screening approach or a non-library based approach to identify nucleic acids that induce gene silencing. These methods involve the direct delivery of in vitro transcribed dsRNA or the delivery of a plasmid that direct the cell to make its own dsRNA.

Short dsRNA or a plasmid encoding short dsRNA may also administered in any of the functional genomics applications if desired to inhibit dsRNA-mediated toxicity, as taught in U.S. Ser. No. 60/375,636 filed Apr. 26, 2002 and U.S. Ser. No. 10/425,006 filed Apr. 28, 2003 “Methods for Silencing Genes Without Inducing Toxicity”, C. Pachuk, the teaching of which is incorporated herein by reference. To avoid problems associated with transfection efficiency, plasmids are designed to contain a selectable marker to ensure the survival of only those cells that have taken up plasmid DNA. One group of plasmids directs the synthesis of dsRNA that is transcribed in the cytoplasm, while another group directs the synthesis of dsRNA that is transcribed in the nucleus.

### Identification of Genes Using Differential Gene Expression

Differential gene expression analysis can be used to identify a nucleic acid sequence that modulates the expression of a target nucleic acid in a cell. Alterations in gene expression induced by gene silencing can be monitored in a cell into which a dsRNA has been introduced. For example, differential gene expression can be assayed by comparing nucleic acids expressed in cells into which dsRNA has been introduced to nucleic acids expressed in control cells that were not transfected with dsRNA or that were mock-transfected. Gene array technology can be used in order to simultaneously examine the expression levels of many different nucleic acids. Examples of methods for such expression analysis are described by Marrack et al. (Current Opinions in Immunology 12:206-209, 2000); Harkin (Oncologist 5:501-507, 2000); Pelizzari et al. (Nucleic Acids Res. 28:4577-4581, 2000); and Marx (Science 289:1670-1672, 2000).

### Identification of Genes by Assaying Polypeptide Biological Activity

Novel nucleic acid sequences that modulate the biological activity of a target polypeptide can also be identified by examining polypeptide biological activity. Various polypeptide biological activities can be evaluated to identify novel genes according to the methods of the invention. For example, the expression of a target polypeptide(s) may be examined. Alternatively, the interaction between a target polypeptide(s) and another molecule(s), for example, another polypeptide or a nucleic acid may be assayed. Phosphorylation or glycosylation of a target polypeptide(s) may also be assessed, using standard methods known to those skilled in the art.

Identification of nucleic acid sequences involved in modulating the biological activity of a target polypeptide may be carried out by comparing the polypeptide biological activity of a cell transfected with a dsRNA to a control cell that has not been transfected with a dsRNA or that has been mock-transfected. A cell that has taken up sequences unrelated to a particular polypeptide biological activity will perform in the particular assay in a manner similar to the control cell. A cell experiencing PTGS of a gene involved in the particular polypeptide biological activity will exhibit an altered ability to perform in the biological assay, compared to the control.

### Example 15

### Design and Delivery of Vectors for Intracellular Synthesis of dsRNA

The utilization of dsRNAs may induce even less toxicity or adverse side-effects when dsRNA resides in certain cellular compartments. Therefore, expression plasmids that transcribe candidate and/or short dsRNA in the cytoplasm and in the nucleus may be utilized. There are two classes of nuclear transcription vectors: one that is designed to express polyadenylated dsRNA (for example, a vector containing an RNA polymerase II promoter and a poly A site) and one that expresses non-adenylated dsRNA (for example, a vector containing an RNA polymerase II promoter and no poly A site, or a vector containing a T7 promoter). Different cellular distributions are predicted for the two species of RNA; both vectors are transcribed in the nucleus, but the ultimate destinations of the RNA species are different intracellular locations. Intracellular transcription may also utilize bacteriophage T7 and SP6 RNA polymerase, which may be designed to transcribe in the cytoplasm or in the nucleus. Alternatively, Qbeta replicase RNA-dependent RNA polymerase may be used to amplify dsRNA. Viral RNA polymerases, either DNA and RNA dependent, may also be used. Alternatively, dsRNA replicating polymerases can be used. Cellular polymerases such as RNA Polymerase I, II, or III or mitochondrial RNA polymerase may also be utilized. Both the cytoplasmic and nuclear transcription vectors contain an antibiotic resistance gene to enable selection of cells that have taken up the plasmid. Cloning strategies employ chain reaction cloning (CRC), a one-step method for directional ligation of multiple fragments (Pachuk et al., Gene 243:19-25, 2000). Briefly, the ligations utilize bridge oligonucleotides to align the DNA fragments in a particular order and ligation is catalyzed by a heat-stable DNA ligase, such as Ampligase, available from Epicentre.

Inducible or Repressible Transcription Vectors

If desired, inducible and repressible transcription systems can be used to control the timing of the synthesis of dsRNA. For example, synthesis of candidate dsRNA molecules can be induced after synthesis or administration of short dsRNA which is intended to prevent possible toxic effects due to the candidate dsRNA. Inducible and repressible regulatory systems involve the use of promoter elements that contain sequences that bind prokaryotic or eukaryotic transcription factors upstream of the sequence encoding dsRNA. In addition, these factors also carry protein domains that transactivate or transrepress the RNA polymerase II. The regulatory system also has the ability to bind a small molecule (e.g., a coinducer or a corepressor). The binding of the small molecule to the regulatory protein molecule (e.g., a transcription factor) results in either increased or decreased affinity for the sequence element. Both inducible and repressible systems can be developed using any of the inducer/transcription factor combinations by positioning the binding site appropriately with respect to the promoter sequence. Examples of previously described inducible/repressible systems include lac, ara, Steroid-RU486, and ecdysone—(Rheogene, Lac (Cronin et al. Genes & Developnment 15: 1506-1517, 2001), ara (Khlebnikov et al., J. Bacteriol. 2000 December; 182(24):7029-34), ecdysone (Rheogene, www.rheogene.com), RU48 (steroid, Wang X J, Liefer K M, Tsai S, O'Malley B W, Roop D R., Proc Natl Acad Sci USA. 1999 Jul. 20; 96(15):8483-8), tet promoter (Rendal et al., Hum Gene Ther. 2002 January; 13(2):335-42. and Larnartina et al., Hum Gene Ther. 2002 January; 13(2):199-210), or a promoter disclosed in WO 00/63364, filed Apr. 19, 2000.

Nuclear Transcription Vectors

Nuclear transcription vectors are designed such that the target sequence is flanked on one end by an RNA polymerase II promoter (for example, the HCMV-IE promoter) and on the other end by a different RNA polymerase II promoter (for example, the SCMV promoter). Other promoters that can be used include other RNA polymerase II promoters, an RNA polymerase I promoter, an RNA polymerase III promoter, a mitochondrial RNA polymerase promoter, or a T7 or SP6 promoter in the presence of T7 or SP6 RNA polymerase, respectively, containing a nuclear localization signal. Bacteriophage or viral promoters may also be used. The promoters are regulated transcriptionally (for example, using a tet ON/OFF system (Forster et al., supra; Liu et al., supra; and Gatz, supra) such that they are only active in either the presence of a transcription-inducing agent or upon the removal of a repressor. A single chromosomal integrant is selected for, and transcription is induced in the cell to produce the nuclear dsRNA.

Those vectors containing a promoter recognized by RNA PolI, RNA PolII, or a viral promoter in conjunction with co-expressed proteins that recognize the viral promoter, may also contain optional sequences located between each promoter and the inserted cDNA. These sequences are transcribed and are designed to prevent the possible translation of a transcribed cDNA. For example, the transcribed RNA is synthesized to contain a stable stem-loop structure at the 5′ end to impede ribosome scanning. Alternatively, the exact sequence is irrelevant as long as the length of the sequence is sufficient to be detrimental to translation initiation (e.g., the sequence is 200 nucleotides or longer). The RNA sequences can optionally have sequences that allow polyA addition, intronic sequences, an HIV REV binding sequence, Mason-Pfizer monkey virus constitutive transport element (CTE) (U.S. Pat. No. 5,880,276, filed Apr. 25, 1996), and/or self splicing intronic sequences.

To generate dsRNA, two promoters can be placed on either side of the target sequence, such that the direction of transcription from each promoter is opposing each other. Alternatively, two plasmids can be cotransfected. One of the plasmids is designed to transcribe one strand of the target sequence while the other is designed to transcribe the other strand. Single promoter constructs may be developed such that two units of the target sequence are transcribed in tandem, such that the second unit is in the reverse orientation with respect to the other. Alternate strategies include the use of filler sequences between the tandem target sequences.

Cytoplasmic Transcription Vectors

Cytoplasmic transcription vectors are made according to the following method. This approach involves the transcription of a single-stranded RNA template in the nucleus, which is then transported into the cytoplasm where it serves as a template for the transcription of dsRNA molecules. The DNA encoding the ssRNA may be integrated at a single site in the target cell line, thereby ensuring the synthesis of only one species of candidate dsRNA in a cell, each cell expressing a different dsRNA species.

A desirable approach is to use endogenous polymerases such as the mitochondrial polymerase in animal cells or mitochondrial and chloroplast polymerases in plant cells for cytoplasmic and mitochondrial (e.g., chloroplast) expression to make dsRNA in the cytoplasm. These vectors are formed by designing expression constructs that contain mitochondrial or chloroplast promoters upstream of the target sequence. As described above for nuclear transcription vectors, dsRNA can be generated using two such promoters placed on either side of the target sequence, such that the direction of transcription from each promoter is opposing each other. Alternatively, two plasmids can be cotransfected. One of the plasmids is designed to transcribe one strand of the target sequence while the other is designed to transcribe the other strand. Single promoter constructs may be developed such that two units of the target sequence are transcribed in tandem, such that the second unit is in the reverse orientation with respect to the other. Alternate strategies include the use of filler sequences between the tandem target sequences.

Alternatively, cytoplasmic expression of dsRNA is achieved by a single subgenomic promoter opposite in orientation with respect to the nuclear promoter. The nuclear promoter generates one RNA strand that is transported into the cytoplasm, and the singular subgenomic promoter at the 3′ end of the transcript is sufficient to generate its antisense copy by an RNA dependent RNA polymerase to result in a cytoplasmic dsRNA species.

### Example 16

### Cloning of Mouse and Human Dicer

To facilitate the in vivo cleavage of expressed or administered dsRNA (e.g., long dsRNA) molecules, dicer protein can be expressed intracellularly. Cloning of the genes for murine and human dicer into a eukaryotic expression vector is performed through a series of reverse transcriptase-polymerase chain reactions (RT-PCRs). The oligonucleotide primers for these RT-PCRs are derived from the published sequences for these genes: GenBank accession number NM_{—}148948 for murine dicer and GenBank accession number NM_{—}030621 for human dicer.

Cloning of the 5754 nucleotide mouse dicer and the 5775 nucleotide human dicer genes is performed through three RT-PCR reactions of approximately 2000 nucleotides each. Exemplary sources of RNA for the RT-PCR reactions include mouse spleen cells and a human cell line such as HuH7. RNA extraction is performed using standard techniques such as described in “Molecular Cloning” (A Laboratory Manual, Second Edition, Sambrook, Fritsch and Maniatis, 1989, Cold Spring Harbor Laboratory Press, NY). The resulting amplicons are designed such that there is approximately 100 nucleotides of overlap between adjacent segments. These segments are then ligated and combined with PCR primers corresponding to the 5′ and 3′ ends of the dicer genes and the entire dicer gene is amplified. The 5′ PCR primer has additional sequences encoded at the 5′ end to serve as a Kozak sequence. The inclusion and design of primers containing these elements is standard and well known to one skilled in the art of designing eucaryotic expression vectors. The amplicon is directionally ligated into a eukaryotic expression vector of choice, such as pcDNA3 from Invitrogen. Directional ligation is performed as described in “Chain reaction cloning: a one-step method for directional ligation of multiple DNA fragments”, Pachuk et al., Gene, 243: pp 19-25, 2000. Alternatively, the 5′ and 3′ PCR primers are designed to contain restriction sites near their 5′termini such that the PCR amplicon contains the entire dicer open reading frame with a Kozak element. Restriction enzyme digestion at these sites enables ligation into compatible sites in any appropriate vector. This type of cloning is standard methodology and is well known to one skilled in the art.

Cloning of the mouse dicer gene may be accomplished using the following oligonucleotides (nucleotide numbers from GenBank NM_{—}148948): mouse RT oligo-1 (nucleotides 6035-6015; 3′ untranslated region, 5′-GTCTTGCCGCCTGTGAGTCCG-3′), mouse forward primer-1 (nucleotides 3995-4015, 5′-CGCTAACACATCTACCTCAGA-3′), mouse reverse primer-1 (nucleotides 6008-5984,5′-TCAGCTGTTAGGAACCTGAGGCTGG-3′), mouse RT oligo-2 (nucleotides 4123-4102, 5′-GTCCTTGAGGAGTACCCAACAG-3′), mouse forward primer-2 (nucleotides 2096-2118, 5′-GTATGTGCTGAGGCCTGATGATG-3′), mouse reverse primer-2 (nucleotides 4096-4076, 5′-CTCTGCTCAGAGTCCATCCTG-3′), mouse RT primer-3 (nucleotides 2222-2202, 5′-GGTTCTACATTTGGGAGCTAG-3′), mouse forward primer-3 (nucleotides 249-272 including native Kozak sequence, 5′-CACTGGATGAATGAAAAGCCCTGC-3′), and mouse reverse primer-3 (nucleotides 2197-2175, 5′-GTAAACGGATCACTTGGTAATCG-3′). Cloning of the human dicer gene may be accomplished using the following oligonucleotides (nucleotide numbers from GenBank NM_{—}030621): human RT-oligo-1 (nucleotides 5963-5943 including six nucleotides from 3′ untranslated region, 5′-GCGGTTTCAGCTATTGGGAAC-3′), human forward primer-1 (nucleotides 3957-3980, 5′-GTGATGGCCGTAATGCCTGGTACG-3′), human reverse primer-1 (nucleotides 5957-5937, 5′-TCAGCTATTGGGAACCTGAGG-3′), human RT-oligo-2 (nucleotides 4080-4060, 5′-GAATAAGTCCAGGATTGGGGC-3′), human forward primer-2 (nucleotides 2056-2076, 5′-CACGAGTCACAATCAACACGG-3′), human reverse primer-2 (nucleotides 4056-4033, 5′-GAGTCCTTGAGGAGTACCCAATAG-3′), human RT-oligo-3 (nucleotides 2182-2157, 5′-GAATAAAATGTACCATCAGGCAACTC-3′), human forward primer-3 (nucleotides 173-197 including native Kozak sequence, 5′-CACTGGATGAATGAAAAGCCCTGC-3′), and human reverse primer-3 (nucleotides 2155-2133,5′-CGGGTTCTGCATTTAGGAGCTAG-3′).

In a typical experiment in cell culture, a dsRNA (e.g., long dsRNA) expression vector is co-transfected into cells with a dicer expression vector. The long dsRNA expression vector encodes dsRNA from, e.g., 40 bp to 10,000 bp, such as desirably 40 bp to 5000 bp. The dsRNA can be in the form of a duplex (i.e., a dsRNA composed of two RNA molecules), or it can be a single molecule of RNA that includes a single hairpin or multiple hairpins. The promoter for dsRNA expression can be, e.g., an RNA pol I, RNA pol II, or RNA pol III promoter. The promoter can be derived from a bacteria, bacteriophage, or virus, such as, but not limited to, T7, SP6, HCMV, or mitochondrial promoters. In some instances, such instances in which a bacteriophage or viral promoter is used, the cognate polymerase is also supplied. This polymerase can be supplied by encoding the polymerase using an expression vector that is co-transfected with the dsRNA expression vector and the dicer expression vector. Alternatively, the polymerase is encoded by the dsRNA or dicer expression vector. The promoters and/or polymerase can be derived from alphaviruses, adenoviruses, AAV, delta virus, pox virus, herpes viruses, papova viruses, poliovirus, pseudorabies virus, retroviruses, lentiviruses, positive and negative stranded RNA viruses, viroids, or virusoids.

In other methods, the dsRNA is encoded by the same vector as dicer. Alternatively, dsRNA is administered (e.g., transfected or injected) into the cell, tissue, or mammal.

### Example 17

### Non-Library Approaches for the Identification of a Nucleic Acid Sequence that Modulates Cell Function, Cellular Gene Expression, or Biological Activity of a Target Polypeptide

Nucleic acid sequences that modulate cell function, gene expression in a cell, or the biological activity of a target polypeptide in a cell may also be identified using non-library based approaches involving PTGS. For example, a single known nucleic acid sequence encoding a polypeptide with unknown function or a single nucleic acid fragment of unknown sequence and/or function can be made into a “candidate” dsRNA molecule. This candidate dsRNA is then transfected into a desired cell type. A short dsRNA or a nucleic acid encoding a short dsRNA is optionally also administered to prevent toxicity. The cell is assayed for modulations in cell function, gene expression of a target nucleic acid in the cell, or the biological activity of a target polypeptide in the cell, using methods described herein. A modulation in cell function, gene expression in the cell, or the biological activity of a target polypeptide in the cell identifies the nucleic acid of the candidate dsRNA as a nucleic acid the modulates the specific cell function, gene expression, or the biological activity of a target polypeptide. As a single candidate dsRNA species is transfected into the cells, the nucleic acid sequence responsible for the modulation is readily identified.

The discovery of novel genes through the methods of the present invention may lead to the generation of novel therapeutics. For example, genes that decrease cell invasion may be used as targets for drug development, such as for the development of cytostatic therapeutics for use in the treatment of cancer. Development of such therapeutics is important because currently available cytotoxic anticancer agents are also toxic for normal rapidly dividing cells. In contrast, a cytostatic agent may only need to check metastatic processes, and by inference, slow cell growth, in order to stabilize the disease. In another example, genes that increase neuronal regeneration may be used to develop therapeutics for the treatment, prevention, or control of a number of neurological diseases, including Alzheimer's disease and Parkinson's disease. Genes that are involved in the ability to support viral replication and be used as targets in anti-viral therapies. Such therapies may be used to treat, prevent, or control viral diseases involving human immunodeficiency virus (HIV), hepatitis C virus (HCV), hepatitis B virus (HBV), and human papillomavirus (HPV). The efficacies of therapeutics targeting the genes identified according to the present invention can be further tested in cell culture assays, as well as in animal models.

### Example 18

### Analysis of RNA from Transfected Cells

Regardless of whether a library based screening approach or a non-library based approach was used to identify nucleic acid sequences, in order to measure the level of dsRNA effector molecule within the cell, as well as the amount of target mRNA within the cell, a two-step reverse transcription PCR reaction is performed with the ABI PRISM™ 7700 Sequence Detection System. Total RNA is extracted from cells transfected with dsRNA or a plasmid from a dsRNA expression library using Trizol and DNase. Two to three different cDNA synthesis reactions are performed per sample; one for human GAPDH (a housekeeping gene that should be unaffected by the effector dsRNA), one for the target mRNA, and/or one for the sense strand of the expected dsRNA molecule (effector molecule). Prior to cDNA synthesis of dsRNA sense strands, the RNA sample is treated with T1 RNase. The cDNA reactions are performed in separate tubes using 200 ng of total RNA and primers specific for the relevant RNA molecules. The cDNA products of these reactions are used as templates for subsequent PCR reactions to amplify GAPDH, the target cDNA, and/or the sense strand copied from the dsRNA. All RNA are quantified relative to the internal control, GAPDH.

### Example 19

### Target Sequence Identification

To identify the target sequence affected by a dsRNA, using any of the above-described methods, DNA is extracted from expanded cell lines (or from the transfected cells if using a non-integrating dsRNA system) according to methods well known to the skilled artisan. The dsRNA encoding sequence of each integrant (or non-integrated dsRNA molecule if using a non-library based method) is amplified by PCR using primers containing the sequence mapping to the top strand of the T7 promoter (or any other promoter used to express the dsRNA). Amplified DNA is then cloned into a cloning vector, such as pZERO blunt (Promega Corp.), and then sequenced. Sequences are compared to sequences in GenBank and/or other DNA databases to look for sequence identity or homology using standard computer programs. If the target mRNA remains unknown, the mRNA is cloned from the target cell line using primers derived from the cloned dsRNA by established techniques (Sambrook et al., supra). Target validation is then carried out as described in more detail, e.g., in U.S. patent application Ser. No. 10/062,707, filed 31 Jan. 2002, incorporated herein by reference, and US20020132257A1: “Use of post-transcriptional gene silencing for identifying nucleic acid sequences that modulate the function of a cell”, published 9-19-2002.

Administration of dsRNA without Inducing a dsRNA-Mediated Stress Response

We have shown that intracellular expression of dsRNA does not induce the RNA stress response. See e.g., US 2002/0132257 A1, published Sep. 19, 2002, “The use of post-transcriptional gene silencing for identifying nucleic acid sequences that modulate the function of a cell”. The cells that were used in these experiments were competent for RNA stress response induction as was demonstrated by the ability of cationic lipid complexed poly(I)(C) and in vitro transcribed RNA to induce/activate all tested components of this response. In addition, the cells were found to be responsive to exogenously added interferon. These results imply that the cells used for these experiments are not defective in their ability to mount an RNA stress response and therefore can be used as predictors for other cells, both in cell culture and in vivo in animal models. This method, which does not induce the interferon stress response, has also been found to effectively induce PTGS. This method therefore provides a method to induce PTGS without inducing an undesired RNA stress response.

Although these results were generated using a vector that utilizes a T7 transcription system and therefore expresses dsRNA in the cytoplasm, the vector system can be changed to other systems that express dsRNA intracellularly. Similar results are expected with these expression systems. These systems include, but are not limited to, systems that express dsRNA or hairpin RNA molecules in the nucleus, in the nucleus followed by transport of the RNA molecules to the cytoplasm, or in the cytoplasm using non-T7 RNA polymerase based expression systems.

Summary

Current evidence indicates that long dsRNA molecules are processed intracellularly into smaller ds ribo-oligonucleotides of 21-24 base-pairs. These ribo-oligonucleotides, termed small interfering RNA molecules (siRNAs), have been implicated as the dsRNA species that effect PTGS. In one aspect of the invention, desirable embodiments use longer dsRNA molecules that can be processed intracellularly into hundreds of different siRNA molecules, many of which should be effective. In another aspect of the invention, desirable embodiments use a series of short dsRNAs (19 to 30 bps, 19 to 27, 21 to 23 basepairs) interspersed by mismatched, single-stranded regions which can be processed by cellular enzymes even without adequate levels of the Dicer enzyme. Other desirable embodiments use dsRNAs which include some single-stranded regions amenable to processing without Dicer as well as longer dsRNA regions which need Dicer for processing to siRNAs.

Summary

An efficient method for inducing long-term gene silencing in mammalian systems has been identified. This method allows for the sustained expression of dsRNA (e.g., long dsRNA) within cells (e.g., vertebrate cells, such as mammalian cells) without invoking the components of the RNA stress or type I interferon response pathway.

We have shown that cytoplasmic expression of long dsRNA does not invoke an RNA stress response and is a very potent inducer of gene silencing. For administration of dsRNA (e.g., long dsRNA), delivery systems other than cationic lipids are desirable. These other delivery systems, such as those described herein, may also prevent an interferon response. Additionally, short dsRNA can be administered to inhibit dsRNA-mediated toxicity as described herein.

Optimization of the Concentrations and Relative Ratios of In Vitro or In Vivo Produced dsRNA and Delivery Agent

If desired, optimal concentrations and ratios of dsRNA to a delivery agent such as a cationic lipid, cationic surfactant, or local anesthetic can be readily determined to achieve low toxicity and to efficiently induce gene silencing using in vitro or in vivo produced dsRNA. Such methods and factors affecting nucleic acid/cationic lipid interactions are described in more detail in US20020132257A1: “Use of post-transcriptional gene silencing for identifying nucleic acid sequences that modulate the function of a cell”, published 9-19-2002. and in Pachuk et al. DNA Vaccines—Challenges in Delivery, Current Opinion in Molecular Therapeutics, 2(2) 188-198, 2000 and Pachuk et al., BBA, 1468, 20-30, (2000)). Furthermore, different lipids, local anesthetics, and surfactants differ in their interactions between themselves, and therefore novel complexes can be formed with differing biophysical properties by using different lipids singularly or in combination. For each cell type, the following titration can be carried out to determine the optimal ratio and concentrations that result in complexes that do not induce the stress response or interferon response. At several of these concentrations PTGS is predicted to be induced; however, PTGS is most readily observed under conditions that result in highly diminished cytotoxicity.

Applications of Present Methods

Short dsRNA molecules can be used in conjunction with exogenously added or endogenously expressed dsRNA molecules in gene silencing applications to prevent the activation of PKR that would otherwise be elicited by the latter dsRNA. Currently, the administration of such exogenously added dsRNA to cells and animals for gene-silencing experiments is limited by the cytotoxicity induced by dsRNA (e.g., long dsRNA). Short dsRNA or a vector stably or transiently expressing short dsRNA can be delivered before (e.g., 10, 20, 30, 45, 60, 90, 120, 240, or 300 minutes before), during, or after (e.g., 2, 5, 10, 20, 30, 45, 60, or 90 minutes after) the delivery of exogenous dsRNA or a vector encoding dsRNA to animals or cell cultures. A vector expressing a short dsRNA can also be administered up to 1, 2, 3, 5, 10, or more days before administration of dsRNA homologous to a target nucleic acid. A vector expressing short dsRNA can be administered any number of days before the administration of dsRNA homologous to a target nucleic acid (e.g., target-specific dsRNA) or a vector encoding this dsRNA, as long as the dsRNA-mediated stress response pathway is still inhibited by the short dsRNA when the target-specific dsRNA is administered. The timing of the delivery of these nucleic acids can be readily be selected or optimized by one skilled in the art of pharmacology using standard methods. See also the teaching of U.S. Ser. No. 60/375,636 filed Apr. 26, 2002 and U.S. Ser. No. 10/425,006 filed Apr. 28, 2003, “Methods for Silencing Genes Without Inducing Toxicity”, C. Pachuk, which is incorporated herein by reference.

### Example 20

### Exemplary Clinical and Industrial Applications of the Constructs and Methods of the Invention

The dsRNA structures, e.g., dsRNA with mismatched regions, one strand with two or more hairpin regions separated by single-stranded regions, including partial and/or forced hairpins, and dsRNA expression constructs of the invention can also be used in methods to treat, stabilize, or prevent diseases associated with the presence of an endogenous or pathogen protein in vertebrate organisms (e.g., human and non-human mammals). These methods are expected to be especially useful for therapeutic treatment for viral diseases, including chronic viral infections such as HBV, HIV, papilloma viruses, and herpes viruses. In some embodiments, the methods of the invention are used to prevent or treat acute or chronic viral diseases by targeting a viral nucleic acid necessary for replication and/or pathogenesis of the virus in a mammalian cell. Slow virus infection characterized by a long incubation or a prolonged disease course are especially appropriate targets for the methods of the invention, including such chronic viral infections as HTLV-I, HTLV-II, EBV, HBV, CMV, HCV, HIV, papilloma viruses, and herpes viruses. For prophylaxis of viral infection, the selected gene target is desirably introduced into a cell together with the short dsRNA and long dsRNA molecules of the invention. Particularly suitable for such treatment are various species of the Retroviruses, Herpesviruses, Hepadnaviruses, Poxviruses, Papillomaviruses, and Papovaviruses. Exemplary target genes necessary for replication and/or pathogenesis of the virus in an infected vertebrate (e.g., mammalian) cell include nucleic acids of the pathogen or host necessary for entry of the pathogen into the host (e.g., host T cell CD4 receptors), nucleic acids encoding proteins necessary for viral propagation (e.g., HIV gag, env, and pol), and regulatory genes such as tat and rev. Other exemplary targets include nucleic acids for HIV reverse transcriptase, HIV protease, HPV6 L1 and E2 genes, HPV11 L1 and E2 genes, HPV16 E6 and E7 genes, HPV18 E6 and E7 genes, HBV surface antigens, core antigen, and reverse transcriptase, HSD gD gene, HSVvp16 gene, HSVgC, gH, gL, and gB genes, HSV ICP0, ICP4, and ICP6 genes; Varicella zoster gB, gC and gH genes, and non-coding viral polynucleotide sequences which provide regulatory functions necessary for transfer of the infection from cell to cell (e.g., HIV LTR and other viral promoter sequences such as HSV vp16 promoter, HSV-ICP0 promoter, HSV-ICP4, ICP6, and gD promoters, HBV surface antigen promoter, and HBV pre-genomic promoter). Desirably, a dsRNA (e.g., long dsRNA) of the invention reduces or inhibits the function of a viral nucleic acid in the cells of a mammal or vertebrate, and a short dsRNA of the invention blocks the dsRNA stress response that may be triggered by dsRNA.

Exemplary retroviral targets include, but are not limited to, HIV-1 and 2, (LTR promoter element) which drives the expression of most or all of the HIV genes gag, integrase, pol, env, vpx, vpr, vif, nef, HTLV-1 and 2, and pro.Exemplary Hepatitis B the promoters include promoters for antigen genes, for core and e antigen, polymerase, and X protein. Exemplary Hepatitis B target genes include genes encoding surface antigen, core and antigen, polymerase, and X protein.

Exemplary Pox viruses include small pox and vaccinia. Some examples of genes and their promoters are the early, intermediate, and late stage promoters; and promoters and coding sequences for RNA polymerase (multi-subunit), Early transcription factor, poly (A) polymerase, capping enzyme, RNA methyltransferase, DNA-dependent ATPase, RNA/DNA-dependent NTPase, DNA topoisomerase I, nicking-joining enzyme, protein kinase 1 and 2, glutaredoxin, C23L-secreted protein, core proteins, virion proteins, membrane proteins and glycoproteins, transcactivators, DNA polymerase, and complement inhibitor.

Exemplary Herpesviruses include HSV-1 and 2, CMV, EBV, and chicken pox. Exemplary promoters for these viruses include the immediate early, early, intermediate and late promoters, and exemplary genes include any gene expressed from these promoters such as those encoding the immediate early proteins including ICP0, ICP4 and ICP6, vp16, capsid proteins, virion proteins, tegument proteins, envelope proteins and glycoproteins including gD and gB, helicase/primase, DNA polymerase, matrix protein, regulatory proteins, protein kinase, and other proteins.

Examples of Human Papillomaviruses include types 1, 2, 3, 4, 5, 6, 8, 11, 13, 16, 18, 31, 33, 35, 39, 41, 42, 47, 51, 57, 58, 63, and 65. Exemplary promoters of interest are those that drive the expression of E6 and E7, E1, E2, E3 and E4 and E5, and L1, and L2, and exemplary genes include the aforementioned genes.

Examples of adenoviral promoters and genes include promoters and coding sequences for E1A, E2A, E4, E2B-TP, E2 Bpol, Iva2, L1-L5, E1B genes, and E3 genes.

Other exemplary viral promoters and genes include promoters and genes of any of the following viruses: parvoviruses, Encephalitic viruses such as West Nile and Japanese encephalitis, Dengue, Yellow fever, Ebola, Marburg, polio, measles, mumps, as well as other viruses in the families of picornaviridae, calciviridae, astroviridae, togaviridae, flaviviridae, coronaviridae, rhabdoviridae, filoviridae, paramyxoviridae, orthomyxoviridae, bunyaviridae, arenaviridae, and reoviridae.

Other exemplary pathogens include bacteria, rickettsia, chlamydia, fungi, and protozoa such as extraintestinal pathogenic protozoa which cause malaria, babesiosis, trypanosomiasis, leishmaniasis, or toxoplasmosis. The intracellular malaria-causing pathogen Plasmodium species P. falciparum, P. vivax, P. ovale, and P. malariae are desirable targets for dsRNA-mediated gene silencing, especially in the chronic, relapsing forms of malaria. Other intracellular pathogens include Babesia microti and other agents of Babesiosis, protozoa of the genus Trypanosoma that cause African sleeping sickness and American Trypanosomiasis or Chagas' Disease; Toxoplasma gondii which causes toxoplasmosis, Mycobacterium tuberculosis, M. bovis, and M avium complex which cause various tuberculous diseases in humans and other animals. Desirably, a dsRNA (e.g., long dsRNA) of the invention reduces or inhibits the function of a pathogen nucleic acid in the cells of a mammal or vertebrate, and a short dsRNA of the invention blocks the dsRNA stress response that may be triggered by dsRNA.

In some methods for the prevention of an infection, a pathogen target gene or a region from a pathogen target gene (e.g., a region from an intron, exon, untranslated region, promoter, or coding region) is introduced into the cell or animal. For example, this target nucleic acid can be inserted into a vector that desirably integrates in the genome of a cell and administered to the cell or animal. Alternatively, this target nucleic acid can be administered without being incorporated into a vector. The presence of a region or an entire target nucleic acid in the cell or animal is expected to enhance the amplification of the simultaneously or sequentially administered dsRNA that is homologous to the target gene. The amplified dsRNA or amplified cleavage products from the dsRNA silence the target gene in pathogens that later infect the cell or animal. Short dsRNA is also administered to the cell or animal to inhibit dsRNA-mediated toxicity.

Similarly, to silence an endogenous target gene that is not currently being expressed in a particular cell or animal, it may be necessary to introduce a region from the target gene into the cell or animal to enhance the amplification of the administered dsRNA that is homologous to the target gene. The amplified dsRNA or amplified cleavage products from the dsRNA desirably prevent or inhibit the later expression of the target gene in the cell or animal. Desirably, short dsRNA is also administered to inhibit toxic effects.

Still other exemplary target nucleic acids encode a prion, such as the protein associated with the transmissible spongiform encephalopathies, including scrapie in sheep and goats; bovine spongiform encephalopathy (BSE) or “Mad Cow Disease”, and other prion diseases of animals, such as transmissible mink encephalopathy, chronic wasting disease of mule deer and elk, and feline spongiform encephalopathy. Prion diseases in humans include Creutzfeldt-Jakob disease, kuru, Gerstmann-Straussler-Scheinker disease (which is manifest as ataxia and other signs of damage to the cerebellum), and fatal familial insomnia. Desirably, a dsRNA (e.g., long dsRNA) of the invention reduces or inhibits the function of a prion nucleic acid in the cells of a mammal or vertebrate, and a short dsRNA of the invention blocks the dsRNA stress response that may be triggered by dsRNA.

The invention also provides compositions and methods for treatment or prophylaxis of a cancer in a mammal by administering to the mammal one or more of the compositions of the invention in which the target nucleic acid is an abnormal or abnormally expressed cancer-causing gene, tumor antigen or portion thereof, or a regulatory sequence. Desirably, the target nucleic acid is required for the maintenance of the tumor in the mammal. Exemplary oncogene targets include ABL1, BRAF, BCL1, BCL2, BCL6, CBFA2, CSF1R, EGFR, ERBB2 (HER-2/neu), FOS, HRAS, MYB, MYC, LCK, MYCL1, MYCN, NRAS, ROS1, RET, SRC, and TCF3. Such an abnormal nucleic acid can be, for example, a fusion of two normal genes, and the target sequence can be the sequence which spans that fusion, e.g., the bcr/abl gene sequence (Philadelphia chromosome) characteristic of certain chronic myeloid leukemias, rather than the normal sequences of the non-fused bcr and abl (see, e.g., WO 94/13793, published Jun. 23, 1994, the teaching of which is hereby incorporated by reference). Viral-induced cancers are particularly appropriate for application of the compositions and methods of the invention. Examples of these cancers include human-papillomavirus (HPV) associated malignancies which may be related to the effects of oncoproteins, E6 and E7 from HPV subtypes 16 and 18, p53 and RB tumor suppressor genes, and Epstein-Barr virus (EBV) which has been detected in most Burkitt's-like lymphomas and almost all HIV-associated CNS lymphomas. The composition is administered in an amount sufficient to reduce or inhibit the function of the tumor-maintaining nucleic acid in the mammal.

The gene silencing methods of the present invention may also employ a multitarget or polyepitope approach. Desirably, the sequence of the dsRNA includes regions homologous to genes of one or more pathogens, multiple genes or epitopes from a single pathogen, multiple endogenous genes to be silenced, or multiple regions from the same gene to be silenced. Exemplary regions of homology including regions homologous to exons, introns, or regulatory elements such as promoter regions and non-translated regions.

The methods of the invention may also be useful in any circumstances in which PKR suppression is desired; e.g., in DNA expression systems in which small amounts of dsRNA may be inadvertently formed when transcription occurs from cryptic promoters within the non-template strand. The present invention is also useful for industrial applications such as the manufacture of dsRNA molecules in vertebrate cell cultures. The present invention can be used to make “knockout” or “knockdown” vertebrate cell lines or research organisms (e.g., mice, rabbits, sheep, or cows) in which one or more target nucleic acids are silenced. The present invention also allows the identification of the function of a gene by determining the effect of inactivating the gene in a vertebrate cell or organism. These gene silencing methods can also be used to validate a selected gene as a potential target for drug discovery or development.

### OTHER EMBODIMENTS

From the foregoing description, it will be apparent that variations and modifications may be made to the invention described herein to adopt it to various usages and conditions. Such embodiments are also within the scope of the following claims.

All publication, patents, and patent applications mentioned in this specification are herein incorporated by reference to the same extent as if each individual publication, patent, or patent application was specifically and individually indicated to be incorporated by reference.

What is claimed is:

1. A substantially pure ribonucleic acid (RNA) molecule comprising a first strand and a second strand that hybridize to each other under physiological conditions to form a double-strand region, said double-strand region comprising one or more mismatched regions that separate said double-strand region into two or more double-stranded segments, and wherein said mismatched regions are capable of cleavage by single-strand ribonucleases.

2. The RNA molecule of claim 1, wherein said first and second strands are joined by a loop to form a hairpin structure.

3. The RNA molecule of claim 2 further comprising one or more hairpin structures.

4. (canceled)

5. The RNA molecule of claim 1 wherein at least a portion of at least one of said double-stranded segments has substantial sequence identity to a target polynucleotide, and wherein said ribonucleic acid molecule is capable of reducing expression of said target polynucleotide, relative to expression of said target polynucleotide in the absence of said ribonucleic acid molecule.

6. The RNA molecule of claim 1 wherein at least one double-strand region comprises at least two mismatched regions that separate said double-stranded region into at least three double-stranded segments.

7. (canceled)

8. The RNA molecule of claim 5, wherein one or more of said double-stranded segments has at least 18 contiguous nucleotides with substantial sequence identity to a target polynucleotide.

9-13. (canceled)

14. The RNA molecule of claim 1, wherein at least one strand of at least one double-stranded region comprises a mismatched base pair.

15. The RNA molecule of claim 1, wherein said first and second strands each comprise at least one mismatched ribonucleic acid base.

16. (canceled)

17. (canceled)

18. The RNA molecule of claim 1, wherein an RNA polynucleotide comprising the first or second strand comprises a 5′ end single-strand overhang comprising at least one nucleotide, wherein said nucleotide does not base-pair with another nucleotide.

19. The RNA molecule of claim 1, wherein the 3′ end of an RNA polynucleotide comprising the first or second strands comprises a single-strand overhang comprising at least one nucleotide, wherein said nucleotide does not base-pair with another nucleotide.

20-22. (canceled)

23. The RNA molecule of claim 2, wherein said loop comprises 4 to 10 nucleotides that do not base-pair with a nucleotide of said RNA molecule.

24. (canceled)

25. The RNA molecule of claim 2 further comprising a mismatched region at the 5′ end of a strand or the 3′ end of a strand, wherein said mismatched region comprises at least one nucleotide that does not base-pair with a nucleotide of said RNA molecule.

26-28. (canceled)

29. The RNA molecule of claim 25, wherein said mismatched region comprises about 10 to about 50 nucleotides.

30. The RNA molecule of claim 25, wherein said RNA molecule comprises a mismatched region at the 5′ end of a strand that covalently links said RNA molecule to a 3′ end of a strand of a second RNA molecule.

31-34. (canceled)

35. The RNA molecule of claim 5, wherein said target gene is selected from the group consisting of a gene within the genome of the cell in which the RNA molecule is expressed (host gene), a gene of a pathogen, or a reporter gene.

36-37. (canceled)

38. The RNA molecule of claim 35, wherein said host gene is associated with a cancer, abnormal cell growth, a disease or disorder, or double-stranded ribonucleic acid (dsRNA)-mediated toxicity.

39-47. (canceled)

48. An expression construct comprising a sequence encoding an RNA molecule of claim 1.

49-52. (canceled)

53. The expression construct of claim 48, further comprising one or more of the following: a promoter, a 5′ initiation sequence, a 3′ termination sequence, a sequence encoding a 5′ hairpin, a sequence encoding a constitutive transport element (CTE) sequence, a sequence encoding an intron sequence, an origin of replication, a sequence encoding a polyadenylation sequence, a sequence encoding a polymerase, or a sequence encoding a selectable marker.

54. The expression construct of claim 53, wherein said promoter is selected from the group consisting of an RNA Pol I promoter, an RNA Pol II promoter, an RNA Pol III promoter, HCMV promoter, the T7 promoter, the Sp6 promoter, the U6 promoter, the RSV promoter, a human mitochondrial light chain promoter, and a human mitochondrial heavy chain promoter.

55. (canceled)

56. (canceled)

57. The expression construct of claim 53, wherein said expression construct further encodes an RNA polymerase.

58-70. (canceled)

71. A pharmaceutical composition comprising the ribonucleic acid molecule of claim 1 and a physiologically acceptable excipient.

72. (canceled)

73. A pharmaceutical composition comprising the construct of claim 48 and a physiologically acceptable excipient.

74. (canceled)

75. A method for reducing or inhibiting expression of a gene in a cell, said method comprising administering a ribonucleic acid (RNA) molecule of claim 1 to said cell, wherein at least a portion of one or more double-stranded regions of said RNA molecule have substantial sequence identity to all or a portion of a first target gene, and wherein following cleavage of said first RNA molecule by a single-stranded RNA-specific RNase to liberate double-stranded regions of said RNA molecule, said liberated double-stranded regions from said RNA molecule having substantial sequence identity to all or a portion of said target gene and capable of reducing expression of said target gene by said cell, relative to expression of said target gene by a cell not administered said RNA molecule.

76. The method of claim 74, wherein said cell is a mammalian cell or a cell of a pathogen.

77-79. (canceled)

80. The method of claim 77, wherein said mammalian cell is infected with a virus and said target gene is a viral gene or an RNA transcribed from said viral gene, wherein said method comprises reducing expression of said viral gene, relative to the expression of a viral gene in a mammalian cell infected with said virus but not administered said RNA molecule, said DNA molecule, or said vector.

81. (canceled)

82. (canceled)

83. A method for treating or preventing a disease or disorder in a mammal, said method comprising administering a first ribonucleic acid (RNA) molecule of claim 1 to said mammal, wherein at least a portion of one or more double-stranded regions of said first RNA molecule have substantial sequence identity to all or a portion of a first target gene, wherein said first target gene encodes a polypeptide associated with said disease or disorder, and wherein following cleavage of said first RNA molecule by a single-stranded RNA-specific RNase to produce liberated double-stranded regions of said first RNA molecule, wherein said liberated double-stranded regions of said first RNA molecule with substantial sequence identity to said first target gene reduce expression of said first target gene by said mammal, relative to expression of said first target gene by a mammal not administered said first RNA molecule, and wherein said reduction of expression of said first target gene treats or prevents said disease or disorder.

84. The method of claim 83, wherein said disease or disorder is cancer, systemic lupus erthyematosis, Alzheimer's disease, or Huntington's disease.

85-87. (canceled)

88. A method for treating or preventing infection of a mammal by a pathogen, said method comprising administering a first ribonucleic acid (RNA) molecule of claim 1 to said mammal, wherein at least a portion of one or more double-stranded regions of said first RNA molecule have substantial sequence identity to all or a portion of a first target gene, wherein said first target gene encodes a polypeptide associated with a biological activity of said pathogen, and wherein following cleavage of said first RNA molecule by a single-stranded RNA-specific RNase to produce liberated double-stranded regions of said first RNA molecule, wherein said liberated double-stranded regions from said first RNA molecule with substantial sequence identity to said first target gene reduces expression of said first target gene in said pathogen or in a cell of said mammal infected with said pathogen, relative to expression of said first target gene in a pathogen, or in a cell of a mammal infected with said pathogen, not exposed to said first RNA molecule, and wherein said reduction of expression of said first target gene treats or prevents said infection.

89. The method of claim 88, wherein said pathogen is selected from the group consisting of a virus, a bacterium, a yeast, a fungus, a protozoan, and a parasite.

90. (canceled)

91. (canceled)

92. The method of claim 88, wherein said mammal is a human.

93-97. (canceled)

98. A method for treating or preventing an immune response by a mammal to a transplanted cell, tissue, or organ, said method comprising administering a first ribonucleic acid (RNA) molecule of claim 1 to said mammal prior to, concurrent with, or following transplantation of said cell, tissue or organ, wherein at least a portion of one or more double-stranded regions of said first RNA molecule have substantial sequence identity to all or a portion of a first target gene, or an RNA molecule transcribed from said first target gene, and wherein said first target gene is associated with an immune response to said transplanted cell, tissue, or organ, and wherein following cleavage of said first RNA molecule by a single-stranded RNA-specific RNase to produce liberated double-stranded regions of said first RNA molecule, wherein said liberated double-stranded regions from said first RNA molecule with substantial sequence identity to said first target gene reduces expression of said first target gene in said mammal, relative to expression of said first target gene in a mammal not administered said first RNA molecule, and wherein said reduction of expression of said gene treats or prevents said immune response.

99. (canceled)

100. (canceled)

101. The method of claim 75, wherein expression of said first target gene is reduced by at least 20%.

102. The method of claim 75, wherein expression of said first target gene is reduced by at least 60%.

103. The method of claim 75, wherein expression of said first target gene is reduced by at least 95%.

104-107. (canceled)
